SEQUENCE LISTING (127 sequences)

SEQ ID NO: 1 - protein, 391 aa, synthetic construct
MGGRDIVDGSEFSPSPVPGSQNVPAPAVKKISQYACQRRTTLNNYNQLFTDALDILAENDELRENEGSALAFMRASSVLKSLPFPITSMKDTEGIPSLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYADILESTFEKFKQPSRKVDALDHFQKCFLILKLDHGRVHSEKSGQQEGKGWKAIRVDLVMSPYDRRAFALLGWTGSRQFERDLRRYATHERKMMLDNHALYDRTKRVFLEAESEEEIFAHLGLDYIEPWERNA

SEQ ID NO: 2 - protein, 509 aa, Homo sapiens
MDPPRASHLSPRKKRPRQTGALMASSPQDIKFQDLVVFILEKKMGTTRRAFLMELARRKGFRVENELSDSVTHIVAENNSGSDVLEWLQAQKVQVSSQPELLDVSWLIECIRAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 3 - protein, 530 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKGKTVTISPLDGKVSKLQKALRVFLEAESEEEIFAHLGLDYIEPWERNA

SEQ ID NO: 4 - protein, 520 aa, Bos taurus
MAQQRQHQRLPMDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 5 - protein, 481 aa, Tinamus guttatus
MERIRAPAAASQRKRQREMQSPNAPCTYEIKFNKFVIFIMQRKMGKTRRAFLMELARRKGFRVESELSDSVTHIVAENNSYLEVLDWLRGQAVRDSSRFELLDISWFTACMEAGRPIELEMKYRLMEQDEFPAVNTCEPEVPSFTVSKVSQYSCQRKTTLNNLNKKFTDAFEIMAENYEFKENEIFSLEFQRAASVLKFLPFPVVRMKDIQGLPCMGDQVRDIIEEIIEEGESSRAKDVLNDERYKSFKQFTSVFGVGVKTSEKWYRMGLRTLEEVKAEKTLKLSKMQRVGLLYYEDLVSRVSEAEVEAVDLIVKKAVHMFLPDALVTVTGGFRRGKKIGHDIDFLITNPGPRDDDDLLHKGLLLYCDIIESTFVKEQRPSRHIDAMDNFQKCFAILKLYQPRADSRSYNTSEEFDTAEVKDWKAIRVDLVISPFEQYAYALLGWTGSKQFGRDLRRYANHEKKMMLDNHALYDKRKVLFI

SEQ ID NO: 6 - protein, 505 aa, Eudromia elegans
MERIRAPAAASQKKRQREMQSPNPPCSYKIKFNKLVIFIMQRKMGKTRRTFLMELARRKGFRVESELSDSVTHIVAENNSYPEVLDWLRGQAVRDSSRFELLDISWFTACMEAGRPVESDVKYRLMEQDDFPTVKTCEPEAPSFAASKVSQYSCQRKTTLNNFNKKFTDAFEIMAENYEFKENEIFSLEFLRAASVLKCLPFPVVRMRDVQGLPCMGDQVRDIIEEIIEEGESSRAKDVLNDERYKSFKQFTSVFGVGVKTSEKWYRMGLRTLEEVKAEKTLKLSKMQRVGLLYYEDLVTCVSEAEANAVGLIVKKAVHTFLPDALVTLTGGFRRGKKIGHDIDFLITNPGPREDDDLLHKGLLLYCDIIESTFVKEQRPSRNVDAMDNFQKCFAILKLHQPPADNRSHSTSEEFDMAAVRDWKAIRVDLVISPFEQYAYALLGWTGSKQFGRDLRRYASHEKKMMLDNHALYDKRKRIFLKAGSEEEIFAHLGLDYVEPWERNA

SEQ ID NO: 7 - protein, 513 aa, Nothoprocta perdicaria
MERIRAPAVASRKKSQREMQSPKPPCSYEIKFNKFVIFIVQRKMGKTRRAFLMELARRKGFRVESELSDSVTHVVAENNSYSEVLDWLRGQAVQESSRFELLDISWFTACMEAGRPVEAEMKYRLLEQDECPAVNTCEPEVPSFTASRVSQYSCQRKTTLNNFNKKFTDAFEIMAENYEFKENEIFSLEFLRAASVLKFLPFPVVRMKDIQGLPCMGDQVRDIIEEIIEEGESSRVKDVLNDERYKSFKQFTSVFGVGVKTSEKWYRMGLRTLEEVTAEKTLKLSKMQKVGLLYYEDLVSCVSEAEADAVGLVVKKAVHTFLPDALVTLTGGFRRGKKIGHDIDFLITNPGPREDDDLLHKVVDLWKKQGLLLYCDIIESTFVKEQRPSRNIDAMDNFQKCFAILKLYQPRVDSESQSTSEEFDMAEVKDWKAIRVDLVISPFEQYAYALLGWTGSKQFGRDLRRYANHEKKMMLDNHALYDRRKRIFLKAGSEEEIFAHLGLDYVEPWERNA

SEQ ID NO: 8 - protein, 505 aa, Nothoprocta ornata
MERMRAPAVASRKKSQREMQSPKPPCSYEIKFNKFVIFIVQRKMGKTRRAFLMELARRKGFRVESELSDSVTHIVAENNSYSEVSDWLRGQAVQESSRFELLDISWFTACMEAGRPVETEMKYRLLEQDECPSMNTCEPEVPSFTASKVSQYSCQRKTTLNNFNKKFTDAFEIMAENYEFKENEIFSLEFLRAASVMKFLPFPVVRMKDIQGLPCMGDQVRDIIEEIIEEGESSRAKDVLNDDRYKSFKQFTSVFGVGVKTSEKWYRMGLRTLEEVKAEKTLKLSKMQKVGLLYYEDLVSCVSEAEADAVGLVVKKAVHTFLPDALVTLTGGFRRGKKIGHDIDFLITNPGPREDDDLLHKGLLLYCDIIESTFVKEQRPSRNIDAMDNFQKCFAILKLYQPRVDSKSQSTSEELDMAEVKDWKAIRVDLVISPFEQYAYALLGWTGSKQFGRDLRRYASHEKKMMLDNHALYDRRKRIFLKAGSEEEIFAHLGLDYVEPWERNA

SEQ ID NO: 9 - protein, 505 aa, Nothoprocta pentlandii
MERMRAPAVASRKKSQREMQSPKPPCSYEIKFNKFVIFIVQRKMGKTRRAFLMELARRKGFRVESELSDSVTHIVAENNSYSEVSDWLRGQAVQESSRFELLDISWFTACMEAGRPVETEMKYRLLEQDECPAVNTCEPEVPSFTASKVSQYSCQRKTTLNNLNKKFTDAFEIMAENYEFKENEIFSLEFLRAASVMKFLPFPVVRMKDIQGLPCMGDQVRDIIEEIIEEGESSRAKDVLNDDRYKSFKQFTSVFGVGVKTSEKWYRMGLRTLEEVKAEKTLKLSKMQKVGLLYYEDLVSCVSEAEADAVGLVVKKAVHTFLPDALVTLTGGFRRGKKIGHDIDFLITNPGPREDDDLLHKGLLLYCDIIESTFVKEQRPSRNIDAMDNFQKCFAILKLYQPRVDSKSQSTSEELDMAEVKDWKAIRVDLVISPFEQYAYALLGWTGSKQFGRDLRRYASHEKMMMLDNHALYDRRKRIFLKAGSEEEIFAHLGLDYVEPWERNA

SEQ ID NO: 10 - protein, 505 aa, Nothocercus julius
MERIRAPAVASQRKRQREMQSPNPSCSYKIKFDKFVIFILQRKMGKTRRAFLMELARRKGFRVESELSDSVTHIVAENNSYLEVLDWLRGQAVQDSSRFELLDISWFTACMEAGRPVETDMKYRLVEQDELSTVNTREPEVPSFTASKVSQYSCQRKTTLNNFNKKFTDAFEIMAENYEFKENEIFSLEFQRAASVLKFLPFPVVRMKDIQGLPCMGDQVRDIIEEIIEEGESSRAKDVLNDERYKSFKQFTSVFGVGVKTSEKWYRMGLRTLEEVKAEKTLKLSKMQRVGLLYYEDLVSCVSEAEVDAVGLIVKKTVHMFLPDALVTVTGGFRRGKKIGHDIDFLITNPGPREDDDLLHKGLLLYCDIIESTFVKEQRPSRNIDAMDNFQKCFAILKLYQPQVDNRSHNTSEEFNTAEVKGWKAIRVDLVISPFEQYAYALLGWTGSKQFGRDLRRYANHEKKMMLDNHALYDRRKRIFLKAGSEEEIFAHLGLDYLEPWERNA

SEQ ID NO: 11 - protein, 505 aa, Nothocercus nigrocapillus
MERIRAPAVVSQRKRQREMQSPNPPCSYEIKFDEFVIFIMQRKMGKTRRAFLMELARRKGFRVESELSDSVTHIVAENNSYLEVLDWLRGQAVQDSSRFELLDVSWFTACMEAGRPVETDMKYRLMEQDELSTVNTCEPEVPSFTASKVSQYSCQRKTTLSNFNKKFTDAFEIMAENYEFKENEVFSLEFQRAASVLKSLSFPVVRMKDIQGLPCMGDHVRDIIEEIIEEGESSRAKDVLNDERYKSFKQFTSVFGVGVKTSEKWYRMGLRTLEEVKAEKTLKLSKMQRVGLLYYEDLVSCVSEAEVDAVGLIVKKTVHMFLPDALVTVTGGFRRGKKIGHDIDFLITNPGPREDDDLLHKGLLLYCDIIESTFVKEQRPSRNIDAMDNFQKCFAILKLYQPRVDNRSHNTSEEFKAAEVKDWKAIRVDLVISPFEQYPYALLGWTGSKQFGRDLRRYANHEKKMMLDNHALYDRRKRIFLKAGSEEEIFAHLGLDYLEPWERNA

SEQ ID NO: 12 - protein, 505 aa, Crypturellus undulatus
MERIRAPAAASQRKRQREMQSPNTPCSYEIKFNTFVIFIMQRKMGKTRRAFLMELARRKGFRVESELSDSVTHIVAENNSYLEVMDWLRGQAVQDSSRFELLDISWFTACMEAGRPVESEVKYRLMDKDEFPTVNTCEPEVPSFTASKVSQYSCQRKTTLNNMNKKFTDAFEIMAENYEFKENEILSLEFQRAASVLKFLPFPVVRMKDIQGLPCMGDQVKDIIEEIIEEGESSRTKDVLNDERYKSFKQFTSVFGVGVKTSEKWYRMGLRTLEEVKAEKTLKLSKMQRVGLLYYEDLVSCVSEAEVDAVGLIVKKAVHTFLPDALVTVTGGFRRGKKIGHDIDFLITNPGPRDDDDLLHKGLLLYCDIIESTFVKEQQPSRNIDAMDNFQKCFAILKLYQPQVDNRSYNTSEECDTAEVKDWKAIRVDLVISPFEQYAYALLGWTGSKQFGRDLRRYAHHEKKMMLDNHALYDRRKRIFLKAGSEEEIFAHLGLDYVEPWERNA

SEQ ID NO: 13 - protein, 505 aa, Crypturellus soui
MERIRAPAAASQRKRQREMQSPNAPCSYEIKFNKFVIFLMQRKMGKTRRAFLMELARRKGFRVESELSDSVTHIVAENNSYLEVMDWLRGQAVQDSSRFELLDISWFTACMEAGRPVESEMKYRLMEQDEFPAVNMCEPEVPSFTVSKVSQYSCQRKTTLNNLNKKFTDAFEIMAENYEFKENEILSLEFQRAASVLKFLPFPVLRMKDIQGLPCMGDQVRHIIEEIIEEGESSRAKDVLNDERYKSFKQFTSVFGVGVKTSEKWYRMGLRTLEEVKAEKTLKLSKMQRVGLLYYEDLVSCVSEAEVDAVGLIVKKAVHTFLPDALVTVTGGFRRGKKIGHDIDFLITNPGPREDDDLLHKGLLLYCDIIESTFVKEQQPSRNIDAMDNFQKCFAILKLYQPQVDNRSYNASEECDTAEVKDWKAIRVDLVISPFEQYAYALLGWTGSKQFGRDLRRYARHEKKMMLDNHALYDSRRKIFLKAGSEEEIFAHLGLDYVEPWERNA

SEQ ID NO: 14 - protein, 504 aa, Serilophus lunatus
MDRIKAPAVISHRKRQKELNSPKLTCGYEIKFSKFVIFIMQRKMGTTRRMFLMELARRNGFRVESELSDSVTHIVAENNSYLEVLDWLKGQAVGDISRFELLDISWLTACMEAGRPVDSEIKYRLMNQCQDPLNTPESEVPSSIARKVSHYSCQRKTTLNNYNKKFTDAFEIMAENFEFKENEIFSLEFLRAASLLKSLPFPVTRMKDIQGLPCIGDQVRDIIEEIIEEGESSRVKEVLNDERYKAFKQFTSIFGVGVKTSEKWYRMGLRTVEELKADKTLKLSKMQKAGFLYYEDLVSCVSKAEADAVTLIVKNTVCTFLPDALVTITGGFRRGKKMGHDIDFLITNPGPREDDELLHKGLLLYCDIIESTFVKEQLPSRKVDAMDHFQKCFTILKLYQPRVDNSSYDMSKKLDMAEVKDWKAIRVDLVITPFEQYAYALLGWTGSKQFGRDLRRYATHEKKMILDNHALYDRKKRIFLKAGSEEEIFAHLGLDYVEPWERNA

SEQ ID NO: 15 - protein, 500 aa, Gouania willdenowi
MLHGAVGFHIRRRSKMEKVPVSMHKDAKFESVRLYLVERKMGSSRRAFLTQLARSKGFIVEDILSDVVTHIVSEDCQASSLWTWLEGHGVKTLPMMQVLDITWFTDSMKQGKPVAVEPRHLIQGSLPMLPDATTSTPVAKVSQYACQRRTTTANSNKIFTDAFEVLAESYEFNEMEGQSLAFRRAASVLRSLPSKIQSLRDAESLPFLGKHSIAVIEEILQCGFSMEVQKILSNERFKTLKLFMSVFGVGLKTAEKWYRKGLLSFSDILAAPGIHLNRMQQNGFLHYGDISRAVSKAEAQALGNIIDETVHAITSDALLTLTGGFRRGKDFGHDVDFIVTTPEMGKEEHLLTAVIESLKHQGLILYCDYRASTFDMSRLPNHKFDAMDHFAKCFLILRLDGSRVEGGLCGAEGDGRGWRAVRVDLVSPPMKQYAFTLLGWTGSKQFERDLRRFAQKERHMLLDNHALYDKTKRELLAATTEQDIFAHLGLQYIEPCQRNA

SEQ ID NO: 16 - protein, 493 aa, Silurus meridionalis
MVPLKRRKVTTEQTATHDSKACKFPETVIFIVERKMGASRKSFLTRLGRSKGFLIEESFSSSITHVVSENNTGDEVHIWIKKQQKEGKASTNLNSVNLLDISWLTESLASGNPVPVQDRHKLKTDPKPSEVTPVIRMKSYACQRRTPLKHHNSFLTEALEILAENAEFSENEGRSVAFRRSASVLKALPHAVRTIDELKDLPCLGEHSLSVIKEILEDGTSREVESTRQSEQFQAMKALTGVFGVGVKTADRWVREGLRSPLDLVDTGQKLNHAQQAGVQYYKDLNTPVTRKEAMVISVIVEKAVKALLPEAIITLTGGFRRGKEVGHDVDFLITHPEEGAEEKLMPKIVNWLEDQGLLLYQKTTRNSYLESKDGPARPPSNMDRFERCFSIFKLEQSKIDSNAHAEASSWRAIRVDLVVSPYSQFAFATLGWTGSKLFERELRRWAGQEKGMSLSSHALYDSKKKCYLQASSEEEIFTHLGLEYIPPSERNA

SEQ ID NO: 17 - protein, 499 aa, Coregonus clupeaformis
MNHAGMLVRVKKRKRPVEAGARGQVEVKFKEVTLYLVERKMGSSRRNFLTSLARSKGFRVEDVLSDEVTHVVAEDNQAEGLLAWLRGHGLRDISRLAVLDISWFTDSMREGRPVTVETRHRIQNTVTDCSPPTAVANVSQYACQRRTTTENHNKIFTDVMEELAESSEFNESKGPCLAFRQAASVLKSLPSAVRCLGAIQGLPWLGEHTKAVMEEILTFGRSFKVEEIRCDERYQALKLFTSVFGVGPKTAEKWYRRGLRSLKEILTEPNIQLNRMQRAGFLYYSDISKAVSKAEAEALGCIIEETVHWIAPDAVLALTGGFRRGKEYGHDVDFLLTVPEMGKEEGLLLHVIDRLRDQGILLYCDHQGSTFDVSKLPSCRFEDMDHFQKCFLILRLEEGQVEGERGLQRDPGDSRGWRAVRVDLVAPPVDRYAFALLGWTGSKQFGRDLRTFARKERQMLLDNHALYDKTKKLFLPATTEEDIFAHLGLEYVEPWQRNA

SEQ ID NO: 18 - protein, 501 aa, Oncorhynchus nerka
MNHAGMLALVKKRKRPVEAGAQGQVEVKFKEVTLYLVERKMGSSRRNFLTSLARSKGFRVEDVLSDDVTHVVAEDNQAEVLWAWLMGHGLRDVSRVAVLDISWFTDSMREGRPVRVETRHCIQNTPTGTDCSPPTAVANVSQYACQRRTTTENHNKIFTDVMEELAESSEFNESKGPCLAFRQAASVLKSLPSAVHCLEAIQGLPCLGEHTKAVMEEILTFGRSFKVEEIRCDERYQALKLFTSVFGVGPKTAEKWYRRGLRSLQEILTEPNIQLNRMQRAGFLYYSDISKAVSKAEAKALGCIIEETVHWIAPDAILALTGGFRRGKEYGHDVDFLLTMPEMGKEEGLLLHVIDRLKDQGILLYCEYQGSTFDVSKLPSCRFEDMDCFQKCFLILRLEQGQVAGERGLQRDPGDSRGWRAVRVDLVAPPVDRYAFALLGWTGSKQFGRDLRTFAQKERLMLLDNHALYDKTKKLCLLATTEEDIFAHLGLEYVEPWQRNA

SEQ ID NO: 19 - protein, 510 aa, Vicugna pacos
MDPQQMAPSGPRKKRPRQVGASVASPPHDIKFQDLVLFILEKKMGTTRRTFLMELARRKGFRVENELSDSVTHIVAENNSGSEVLEWLQVQNIKAGSQLELLDVSWLIESMEAGKPVEMTGKHQLVVKTDCSGSPNPGFQKTPPLAVKKISQYACQRRTTLNNCNHIFTDAFEVLAENYEFRENEIFSVTFLRAASVLKSLPFTIISMKDTEGIPCLENKVKCIIEEIIEDGESSEVKAVLNDERYQSFKLFTSVFGVGLKTSEKWFRMGFRTLSKIRSDETLKFTRMQKAGFLYYEDLVSCVTRAEAEAVSVLVKEAVWAFLPDAFVTMTGGFRRGKQIGHDVDFLITSPGTTEAEEEQLLPKVINLWERKELLLYCDLVESTFEKSKLPSRKVDALDHFQKCFLILKLHHQRVDSGLSRRQEGKTWKAIRVDLVMCPYEHRAFALLGWTGSKQFERDLRRYATHERKMILDNHALYDKTKRIFLKAESEEEIFAHLGLDYIEPWERNA

SEQ ID NO: 20 - protein, 512 aa, Elephantulus edwardii
MDPLQMACTGPRKKRARQMDTSMGSTQDIKFQDLVLFILEKKMGTTRRAFLMELARRKGFRVENELSDSVTHIVAENNSGSDVLEWLQVQKIKASSQLELLDVSWLIECMGAGKLVEITGKHQLVSIMVRGDCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 21 - protein, 475 aa, Galeopterus variegatus
MDLLPMAHSGPRKKRPRQTGTSMASTSHDIKFQDLVLFILEKKMGTTRRAFLMELARRKGFRVDNELSDSVTHIVAENNSGSDVLEWLQVQKIRASSQLELLDVSWLIECMRAGKPVEMTGKHQLVVKRDYLASPNPGPQKTLPLAAQKISPYACQRRTTLNNCNHMFTDAFEILAENYEFRENEDSYLAFKRAASVLKSLPFTIISMRDTEGIPCLGDNVKCVIEEIIEDGESSEVKNVLNDERYQSFKLFTSVFGVGLKTSERWFRMGFRTLSTIRSDKSLKFTRMQKAGFLYYEDLVSCVTRAEAEAVSVLVKEAVWAFLPDALVTMTGGFRSQGLLLYHDLVESTFEKLKLPSRNVDALDHFQKCFLILKLDHQRVDSDKSNQQEGKTWKAIRVDLVMCPYECRAFALLGWTGSPQFERDLRRYATHERKMRLDNHALYDKTKRIFLKAESEEEIFAHLGLDYIEPWERNA

SEQ ID NO: 22 - protein, 345 aa, Engystomops pustulosus
DAFDILAENFEFEINKSPLVAFRRASSVLKSLQSPIMSTKDLEGLPLIGVEMKYIIEEILEEGQCCRVLEVLNSERYKAFKAFTSIFGVGLKTSEKWFRMGLRTLEDIKYKKDLKLTTMQKHGLLYYDDIADYVSKMEAEAVEHLMKNIIYKFVPDAIVTITGGFRRGKELGHDVDLLITCPRKGVEREILHNTLNVLKNQGQLLFYDIYESTFDDTKRPSRRVDALDHYQKSFLILKLSRKEVKAEKQRDDGWTRLEDLRDWKAVRVDLVITPHEQYAFALLGWTGSPQFERDLRRYATQEKKMMLDNHALFDKTKNVFLKARSEEDIFAHLGLDYVLPEERNA

SEQ ID NO: 23 - protein, 512 aa, Bufo bufo
MELVRTPLLVPIKKKPKLLHISSSFYHYEVKFKGIVLYLLERKMGASRRNFLMELGRKRGFQIESELRDSVTHIVAENNSGDDVLEWLQTTKLTKANPYQILDISWFTECMGAGYPINIECRHRLQVQQDCSASFNPPLSSSCVHVSPYACQRRTPLQDINRIFTDALDTLAENYEFWENKGPLVAFRRASSVLKSLQSPIVSIKDLRGLPLLGDEMKHIIEEILEEGKCWRVLEVVNSERYNAFKVFTSIFGVGLKTAEKWHRMGFRTLEDIKTKKDLKLTRMQKHGLLYYEDITSHVSKMEADAVEHLVKNIICEFVPDAIVTITGGFRRGKDVGHDVDLLITCPRRGAEKNILHNTINVLKNQGRLLFYDIIESTFDDTKLPSRRVDALDHYQKSFIILKLLKKEVKAQIPRDYGWTEPEDTYPRDWKAVRVDLVISPQEQYAFALLGWTGSPQFERDLRRYASQEKKMMLDNHALFDKTKHIFMRARTEQDIFDHLGLDYVEPGDRNA

SEQ ID NO: 24 - protein, 512 aa, Bufo gargarizans
MELVRTPTLVPIKKKPKLLQVSSSFYHYEVKFKGIVLYLLERKMGASRRNFLMELGRKRGFQIESELRDSVTHIVAENNSGDEVLEWLQTTKLTKANLYQILDISWFTECMGAGYPIIIECRHRLQVQQDCSASFNPPLSSSCVHVSPYACQRQTPLQDINRIFTDALDTLAENYEFLENKGPLVAFRRASSVLKSLQSPIVSIKDLRGLPLLGDEMKHIIEEILEEGKCWRVLEVVNSERYKAFKVFTSIFGVGLKTAEKWHRMGFRTLEDIKTKKDLKLTRMQKHGLLYYEDITSHVSKMEADAVEHLVKNIICEFVPDAIVTITGGFRRGKDVGHDVDLLITCPRRGAEKNILHNTINVLKNQGRLLFYDIIESTFDDTKLPSRRVDALDHYQKSFIILKLLKKEVKAQIPEDDGWTEPEDTYPRDWKAVRVDLVISPQEQYAFALLGWTGSPQFERDLRRYASQEKKMMLDNHALFDKTKHIFLRARTEQDIFDHLGLDYLEPGDRNA

SEQ ID NO: 25 - protein, 570 aa, Sciurus carolinensis
MTLNWVLVLVLHQAIGLVITHRGSHTPAFRDAKDQARHVRITHELEEVTQNLLPCTKQRNESQVGIATIGSSDSVTHIVAENNSGSDVLEWLQVQNIKASSQLELLDISWLIECMRAGRPVETTGKHQLVRRADSAHSNPDPQRIPAHAVRKISQYACQRRTTLDNHNHVFTDAFEVLAENYEFRENEGCYLIFMRAASVLKSLPFTISSMKDIEGIPCLGDKVKCIIEEIIEDGESSEVKAVLNDERYKSFKLFTSVFGVGLKTSERWFRMGFRSLSKIRSDKSLKFTHMQKAGRGHRTPKHNGKAWEMLRKSQARNAAGPLRPGSNRVSLCLEFPPGLCAGWEGGNEGGQAGSSRGHGSEPSPTDKPDPDGLAQAQRRKPTCTDAEARALVQRDHRAPPCKASKPRRLDWCSLVLLYPFSYPFSVDRTCEGLLLYCDLVESTFEKLKIPSRKVDALDHFQKCFLILKLDHQRVDSDKASQQDGKTWKAIRVDLVMCPYERRAFALLGWTGSPQFERDLRRFATHERKMVLDNHALYDKTKRIFLKAESEEEIFAHLGLDYIEPWERNA

SEQ ID NO: 26 - protein, 508 aa, Nanorana parkeri
MDLVRGSGLIPVKKRPKILHGSSSFCHYEVKFKGIVLFLVERKMGTSRRNFLMELAKKRGFQIERELRDCVTHIVAENNSGAEVVEWLQSRKLNNAVKCQIVDISWFTECMGAGCPIKIESRHQLTVQEDCPASFNMPVSSSCVRVSPYACQRRTPLQDINRIFTDAFDILAENYEFLENKGPRVAYQRASAVLKSLPFPILTMKDLEGLPHLGEEMKYIIEEILEDGKSSRVDQVIYSERYKSFKIFTSIFGVGLKTAEKWYRMGYRTLEDITSNKGLTFTSMQKHGLLYYEDITSSVTKTEADAVEHLIRTVICGFVSDAIVTVTGGFRRGKDVGHDVDILITCPQKRDENQILHKTINALKKRDLLLFCYLVESTFDDTKRPSRYIDSLDHFQKCFLILQLQKEESKNAYSHKPKGCDAKRSWKAIRVDLVVTPYEQYAFALLGWTGSQQFERDLRRYASHEKKMMLDNHAMYDKSKNIFLKAETEEDIFTHLGLEYLEPWERNA

SEQ ID NO: 27 - protein, 495 aa, Carassius auratus
MFPKALPTRKRRRTEVASVGGREEVKFRDVTLFLVEKRMGKSRRNFLSNLARSKGFCVDDALSADVTHVVSEGNAAQELWSWLEEQGFREMQDKHVLHISWFTESMSAGRPVPPQIRHYIQNPAVDQRSRTNPSAKTESALSPYACQRRTTMDNHNKIFTDALEVLAENLEFGGNQGPSLAFRRAASVLKSLPAALKHLQETQHLPCLGEHSRAVIEEIIEYGSSSRVEEILNDERYRTMKLFSSVFGVGPKTSESWFCRGLRSFEQVLNEPSVRLNRMQTAGFLFYEDISKPVSRTEASALKIIVEEAVISVNPSATVSITGGFRRGKEFGHDVDFIIKTPEAGQEDAILTAVIERFKSQNILLYSDFQESTFDLRQLPTHCFEAMDHYSKCFLIIKLKREQETDARAGRDWRAVRVDLVAPPLERFAYALLGWTGSTLFERDLRRFARLERGKLLDNHTLYDKATNTFLPANTEEDIFAHLGLEYIEPWQRNA

SEQ ID NO: 28 - protein, 491 aa, Pimephales promelas
MLPTRKRRPEASFPRGHDEVKFKDLTLYLVERRMGKSRRNFLSNLARSKGFCVDDALSGNVTHVVSEGNAAQDLWHWLEEQGFGETPHTHVLDVSWFTESMSAGRPVLVELRHYIQNPAVDHRSCAHPSGKPESAVSAYACQRRTTLDNHNKIFTDALEVLAENMEFVGNRGPCLAFRRAASVLKSLRAPLQHLEETQRLPCLGEHSKAVLEEIFECGSSSRAEDTLNDERYRTMKLFSSVFGVGPRTAESWYCRGLRTFEQVLTEPSIRLNRMQTAGFLFYEDISKPVSRTEASALKIIVEEAVMCVNPHATVHITGGFRRGKEFGHDVDFIIKTPEPGQENAVLPALIQRFKAQNILLYSDFQDSTFDLSQLPTHRFEAMDHFSKCFLIVKLKREQESGEREASRDWRAVRVDLVAPPLERYAYALLGWTGSTLFERDLRRFARLERGKLLDNHALYDKATKAFLPACTEEEIFAHLGLEYIEPWQRNA

SEQ ID NO: 29 - protein, 500 aa, Chanos chanos
MLQSPFLPPVRKRRRPEMTPARVQHEVRFEGVTLYLVERRMGSSRRNFLTHLARSKGFCVHDILSSEVTHIVAEGNPAEELWSWLEEHHLRDMCNLHVLDIRWFTESMQAGRPVAVETRHYIQKPSSKLKTALPATSKPTVSQYACQRRTTLDNQNKIFTDAFEVLAESNEFLDSRGQCLAFRRAASVLKSLQFTLRSLDDIEGLPCLGEQIKSVIEEIFEFRTCSKVEEILRDEKYQTLKLFTSVFGVGPKTAEKWYRKGLRTLKAVLAEPDIHLNKMQEAGFLYYEDISKSVSKADAQAVETLIKDTISYYSSDTKLFLTGGFRRGKEHGHDVDFILTTPEIGKEQALLLFLIDKFKNLGILLYSDFQESTFDIQKLPSCKFEAMDHFPKCFLILKLKTGEQTAGVQQNQNDSRDWKAIRVDLVAPPADRYAFAMLGWTGSTQFERDLRRYARKERRMLLDNHALYDKTKHAFLEAQTEEEIFDLLGLEYIEPWQRNA

SEQ ID NO: 30 - protein, 506 aa, Python bivittatus
MNKMKTSDFSPMRKRQKRMQIAAPLSSYKIEFKDIIIFIVERKMGMTRRMFLMDLARRKGFRVENELSDLVTHVVAENNSCSEILKWLQKHNVEDSSRFRILDIRWLTACMEVGRPVDSEKYQLPEDEDRSVTSDLDRDSISEYACQRRTTLKNYNQKFTDAFEILAENYEFNENKGFCTAFRRAASVLKCLPFTIVQVHDIEGVPWMGKQVKGIIEDIIEEGESSKVKAVLDNENYRSVKLFTSVFGVGLKTSDKWYRMGLRTLEEVKRDKNLKLTRMQKAGFLHYDDLTSCVSKAEADAASLIVQDVVWKIVPNAIVTIAGGFRRGKQTGHDVDFLITVPGSKQEEEELLHTVIDIWKKQELLLYYDLIESTFEDTKLPSRKVDALDHFQKCFAILKVHKEREDKGNSIRSKAFSEEEIKDWKAIRVDLVVVPFEQYAFALLGWTGSTQFERDLRRYATHEKKMMLDNHALYDKTKKIFLNAASEEEIFAHLGLDYLEPWERNA

SEQ ID NO: 31 - protein, 487 aa, Danio rerio
MLPSRKRVRAEVSTSQGGEHVKFSDVTLFLVEKRMGKSRRTFLSSLARSKGFCVDDALSGAVTHVVSEGLSAQDLWLWLEDQGFQETHSKHVLNISWFTESMSAGRPLPVEDTHCIQNPAADQRSCVHLSATPESAVSPYACQRRTTLENHNKIFTEALEVLALNSEFSGNQGSCLGFRRAASVLKSLPAALRSPEEALRLPCLGDNSRAVLEEICECGSSSRVEEIQNDQRYRTMKLFCSVFGVGPKTAESWFCRGLKTFEQVLTEPSIRLNRMQTAGFLFYEDISVPVSRAEAAALKMMMEEALLFINPSATVTITGGFRRGKEFGHDVDFIIKAPEGQEDRILPAVIKRFKSQNVLLYSDFQKSTFDLRQLPNHRFEAMDRFSKCFLLVKLQMQESRTGRNWRAVRVDLVAPPLERFPYALLGWTGSTLFERDLRRFARLERGKLLDNHTLYDKTTKTFLPANTEEDIFQHLGLEYIEPWQRNA

SEQ ID NO: 32 - protein, 391 aa, synthetic construct
MGGRDIVDGSEFSPSPVPGSQNVPAPAVKKISQYAVQRRTTLNNYNQLFTDALDILAENDELRGNEGRALAFMRASSVLKSLPFPITSMKDTEGIPSLGDKVKSIIEEIIEDGESSEVKAVLNDERYKSFKLFTSVFGVGLKTAEKWFRMGFRTLSEIQSDKSLRFTQMQKAGFLYYEDLVSCVNRPEAEAVSMLVKEAVVTFLPDALVTMTGGFRRGKMTGHDVDFLITSPEATEDEEQRLLHKVTDFWKQQGLLLYADILESTFEKFKQPSRKVDALDHFQKAFLILKLDHGRVHSEKSGQQEGKGWKAIRVDLVMSPYDRRAFALLGWTGSRQFNRDLRRYATHERKMMLDNHALYDRTKRVFLEAESEEEIFAHLGLDYIEPWERNA

SEQ ID NO: 33 - protein, 391 aa, synthetic construct
MGGRDIVDGSEFSPSPVPGSQNVPAPAVKKISQYAVQRRTTLNNYNQLFTDALDILAENDELRGNEGRALAFMRASSVLKSLPFPITSMKDTEGIPSLGDKVKSIIEEIIEDGESSEVKAVLNDERYKSFKLFTSVFGVGLKTAEKWFRMGFRTLSEIQSDKSLRFTQMQKAGFLYYEDLVSCVNRPEAEAVSMLVKEAVVTFLPDALVTMTGGFRRGKMTGHDVDFLITSPEATEDEEQRLLHKVTDFWKQQGLLLYADILESTFEKFKQPSRKVDALDHFQKAFLILKLDHGRVHSEKSGQQEGKGWKAIRVDLVMSPYDRRAFALLGWTGSRQFERDLRRYATHERKMMLDNHALYDRTKRVFLEAESEEEIFAHLGLDYIEPWERNA

SEQ ID NO: 34 - protein, 391 aa, synthetic construct
MGGRDIVDGSEFSPSPVPGSQNVPAPAVKKISQYAVQRRTTLNNYNQLFTDALDILAENDELRENEGRALAFMRASSVLKSLPFPITSMKDTEGIPSLGDKVKSIIEEIIEDGESSEVKAVLNDERYKSFKLFTSVFGVGLKTAEKWFRMGFRTLSEIQSDKSLRFTQMQKAGFLYYEDLVSCVNRPEAEAVSMLVKEAVVTFLPDALVTMTGGFRRGKMTGHDVDFLITSPEATEDEEQRLLHKVTDFWKQQGLLLYADILESTFEKFKQPSRKVDALDHFQKAFLILKLDHGRVHSEKSGQQEGKGWKAIRVDLVMSPYDRRAFALLGWTGSRQFNRDLRRYATHERKMMLDNHALYDRTKRVFLEAESEEEIFAHLGLDYIEPWERNA

SEQ ID NO: 35 - protein, 391 aa, synthetic construct
MGGRDIVDGSEFSPSPVPGSQNVPAPAVKKISQYAVQRRTTLNNYNQLFTDALDILAENDELRGNEGRALAFMRASSVLKSLPFPITSMKDTEGIPSLGDKVKSIIEEIIEDGESSEVKAVLNDERYKSFKLFTSVFGVGLKTAEKWFRMGFRTLSEIQSDKSLRFTQMQKAGFLYYEDLVSCVNRPEAEAVSMLVKEAVVTFLPDALVTMTGGFRRGKMTGHDVDFLITSPEATEDEEQRLLHKVTDFWKQQGLLLYADILESTFEKFKQPSRKVDALDHFQKCFLILKLDHGRVHSEKSGQQEGKGWKAIRVDLVMSPYDRRAFALLGWTGSRQFNRDLRRYATHERKMMLDNHALYDRTKRVFLEAESEEEIFAHLGLDYIEPWERNA

SEQ ID NO: 36 - protein, 391 aa, synthetic construct
MGGRDIVDGSEFSPSPVPGSQNVPAPAVKKISQYAVQRRTTLNNYNQLFTDALDILAENDELRGNEGRALAFMRASSVLKSLPFPITSMKDTEGIPSLGDKVKSIIEEIIEDGESSEVKAVLNDERYKSFKLFTSVFGVGLKTAEKWFRMGFRTLSEVQSDKSLRFTQMQKAGFLYYEDLVSCVNRSEAEAVSMLVKEAVVTFLPDALVTMTGGFRRGKMTGHDVDFLITSPEATEDEEQRLLHKVTDFWKQQGLLLYANILESTFEKFKQPSRKVDALDHFQKAFLILKLDHGRVHSEKSGQQEGKGWKAIRVDLVMPPYDRRAFALLGWTGSRQFNRDLRRYATHERKMMLDNHALYDRTKRVFLEAESEEEIFAHLGLDYIEPWERNA

SEQ ID NO: 37 - protein, 391 aa, synthetic construct
MGGRDIVDGSEFSPSPVPGSQNVPAPAVKKISQYAVQRRTTLNNYNQLFTDALDILAENDEFRGNEGSALAFQRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFLYYEDLVSCVNRAEAEAVSMLVKEAVVTFLPDALVTMTGGFRRGKMTGHDVDFLITSPEATEDEEQRLLHKVTDFWKQQGLLLYANILESTFEKFKLPSRKVDALDHFQKGFLILKLDHQRVHSEKSGQQEGKGWKAIRVDLVMPPYDRRAFALLGWTGSRQFNRDLRRYATHERKMLLDNHALYDRTKRVFLEAESEEEIFAHLGLDYIEPWERNA

SEQ ID NO: 38 - protein, 391 aa, synthetic construct
MGGRDIVDGSEFSPSPVPGSQNVPAPAVKKISQYAVQRRTTLNNYNQLFTDALDILAENDELRENEGRALAFMRASSVLKSLPFPITSMKDTEGIPSLGDKVKSIIEEIIEDGESSEVKAVLNDERYKSFKLFTSVFGVGLKTAEKWFRMGFRTLSEIQSDKSLRFTQMQKAGFLYYEDLVSCVNRPEAEAVSMLVKEAVVTFLPDALVTMTGGFRRGKMTGHDVDFLITSPEATEDEEQRLLHKVTDFWKQQGLLLYADIAESTFEKFKQPSRKVDALDHFQKAFLILKLDHGRVHSEKSGQQEGKGWKAIRVDLVMSPYDRRAFALLGWTGSTQFERDLRRYATHERKMMLDNHALYDRTKRVFLEAESEEEIFAHLGLDYIEPWERNA

SEQ ID NO: 39 - protein, 391 aa, synthetic construct
MGGRDIDDGSEFSPSPVPGSQNVPAPAVKKISQYAVQRRTTLNNYNQLFTDALDILAENDEFRENEGSALAFQRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSCVTRPEAEAVSVLVKEAVVTFLPDALVTMTGGFRRGKMTGHDVDFLITSPEATEDEEQRLLHKVTDFWKQQGLLLYADILESTFEKFKLPSRKVDALDHFQKGFLILKLDHQRVHSEESGQQEGKGWKAIRVDLVMPPYDRRAFALLGWTGSRQFERDLRRYATHERKMLLDNHALYDKTKRVFLGAESEEEIFAHLGLDYIEPWERNA

SEQ ID NO: 40 - protein, 391 aa, synthetic construct
MGGRDIVDGSEFSPSPVPGSQNVPAPAVKKISQYAVQRRTTLNNYNQLFTDALDILACNDELRGNEGRALAFMRASSVLKSLPFPITSMKDTEGIPSLGDKVKSIIEEIIEDGESSEVKAVLNDERYKSFKLFTSVFGVGLKTAEKWFRMGFRTLSEIQSDKSLRFTQMQKAGFLYYEDLVSGVNRPEAEAVSMLVKEAVVTFLPDALVTMTGGFRRGKMTGHDVDFLITSPEATEDEEQRLLHKVTDFWKQQGLLLYADILESTFEKFKQPSRKVDALDHFQKAFLILKLDHGRVHSEKSGQQEGKGWKAIRVDLVMSPYDRRAFALLGWTGSTQFERDLRRYATHERKMMLDNHALYDRTKRVFLEAESEEEIFAHLGLDYIEPWERNA

SEQ ID NO: 41 - protein, 391 aa, synthetic construct
MGGRDIVDGSEFSPSPVPGSQNVPAPAVKKISQYAVQRRTTLNNYNQLFTDALDILACNDELRENEGRALAFMRASSVLKSLPFPITSMKDTEGIPSLGDKVKSIIEEIIEDGESSEVKAVLNDERYKSFKLFTSVFGVGLKTAEKWFRMGFRTLSEIQSDKSLRFTQMQKAGFLYYEDLVSGVNRPEAEAVSMLVKEAVVTFLPDALVTMTGGFRRGKMTGHDVDFLITSPEATEDEEQRLLHKVTDFWKQQGLLLYADIAESTFEKFKQPSRKVDALDHFQKAFLILKLDHGRVHSEKSGQQEGKGWKAIRVDLVMSPYDRRAFALLGWTGSTQFERDLRRYATHERKMMLDNHALYDRTKRVFLEAESEEEIFAHLGLDYIEPWERNA

SEQ ID NO: 42 - protein, 391 aa, synthetic construct
MGGRDIDDGSEFSPSPVPGSQNVPAPAVKKISQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 43 - protein, 391 aa, synthetic construct
MGGRDIVDGSEFSPSPVPGSQNVPAPAVKKISQYAVQRRTTLNNYNQLFTDALDILAENDELRENEGRALAFCRASSVLKSLPFPITSMKDTEGIPSLGDKVKSIIEEIIEDGESSEVKAVLNDERYKSFKLFTSVFGVGLKTAEKWFRMGFRTLSEIQSDKSLRFTQMQKAGFLYYEDLVSGVNRPEAEAVSMLVKEAVVTFLPDALVTMTGGFRRGKMTGHDVDFLITSPEATEDEEQRLLHKVTDFWKQQGLLLYADIAESTFEKFKQPSRKVDALDHFQKAFLILKLDHGRVHSEKSGQQEGKGWKAIRVDLVMSPYDRRAFALLGWTGSTQFERDLRRYATHERKMMLDNHALYDRTKRVFLEAESEEEIFAHLGLDYIEPWERNA

SEQ ID NO: 44 - protein, 391 aa, synthetic construct
MGGRDIDDGSEFSPSPVPGSQNVPAPAVKKISQYAVQRRTTLNNYNQLFTDALDILAENDEFRGNEGSALAFQRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRCGKMTGHDVDFLITSPEATEDEEQRLLHKVTDFWKQQGLLLYANILESTFEKFKLPSRKVDALDHFQKGFLILKLDHQRVHSEESGQQEGKGWKAIRVDLVMPPYDRRAFALLGWTGSRQFNRDLRRYATHERKMLLDNHALYDKTKRVFLGAESEEEIFAHLGLDYIEPWERNA

SEQ ID NO: 45 - protein, 391 aa, synthetic construct
MGGRDIDDGSEFSPSPVPGSQNVPAPAVKKISQYAVQRRTTLNNYNQLFTDALDILAENDEFRGNEGSALAFQRASSVLKSLPFPITSMEDLEGIPSLGDEVKSIIEEIIEDGESSEVMAVLNDERYKSFKLFTSVFGVGLKTAKKWYRMGFRTLSEVQSDKSLRFTEMQKAGFKYYEDLVSCVTRPEAEAVSVLVKEAVVTFLPDALVTMTGGFRRGKMTGHDVDFLITSPEATEDEEQRLLHKVTDFWKQQGLLLYANILESTFEKFKLPSRKVDALDHFQKGFLILKLDHQRVHSEESGQQEGKGWKAIRVDLVMPPYDRRAFALLGWTGSRQFNRDLRRYATHERKMLLDNHALYDKTKEVFLGAESEEEIFAHLGLDYIEPWERNA

SEQ ID NO: 46 - protein, 391 aa, synthetic construct
MGGRDIDDGSEFSPSPVPGSQNVPAPAVKKISQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALAHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 47 - protein, 391 aa, synthetic construct
MGGRDIDDGSEFSPSPVPGSQNVPAPAVKKISQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALNHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 48 - protein, 391 aa, synthetic construct
MGGRDIDDGSEFSPSPVPGSQNVPAPAVKKISQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLANHALYDKTKRVFLGAESEEEIFAHLGLDYIEPWERNA

SEQ ID NO: 49 - protein, 391 aa, synthetic construct
MGGRDIDDGSEFSPSPVPGSQNVPAPAVKKISQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLLNHALYDKTKRVFLGAESEEEIFAHLGLDYIEPWERNA

SEQ ID NO: 50 - protein, 391 aa, synthetic construct
MGGRDIDDGSEFSPSPVPGSQNVPAPAVKKISQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNAALYDKTKRVFLGAESEEEIFAHLGLDYIEPWERNA

SEQ ID NO: 51 - protein, 391 aa, synthetic construct
MGGRDIDDGSEFSPSPVPGSQNVPAPAVKKISQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNVALYDKTKRVFLGAESEEEIFAHLGLDYIEPWERNA

SEQ ID NO: 52 - protein, 391 aa, synthetic construct
MGGRDIDDGSEFSPSPVPGSQNVPAPAVKKISQYAVQRRTTLNNYNQLFTDALDILAENDCFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 53 - protein, 391 aa, synthetic construct
MGGRDIDDGSEFSPSPVPGSQNVPAPAVKKISQYAVQRRTTLNNYNQLFTDALDILAENDEFRENCGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 54 - protein, 391 aa, synthetic construct
MGGRDIDDGSEFSPSPVPGSQNVPAPAVKKISQYAVQRRTTLNNYNQLFTDALDILAENDEFRENEGSCLAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 55 - protein, 391 aa, synthetic construct
MGGRDIDDGSEFSPSPVPGSQNVPAPAVKKISQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFCSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 56 - protein, 391 aa, synthetic construct
MGGRDIDDGSEFSPSPVPGSQNVPAPAVKKISQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGCKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 57 - protein, 391 aa, synthetic construct
MGGRDIDDGSEFSPSPVPGSQNVPAPAVKKISQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVCALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 58 - protein, 391 aa, synthetic construct
MGGRDIDDGSEFSPSPVPGSQNVPAPAVKKISQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDCLDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 59 - protein, 391 aa, synthetic construct
MGGRDIDDGSEFSPSPVPGSQNVPAPAVKKISQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSCQFERDLRRYATHERKMLLDNHALYDKTKRVFLGAESEEEIFAHLGLDYIEPWERNA

SEQ ID NO: 60 - protein, 391 aa, synthetic construct
MGGRDIDDGSEFSPSPVPGSQNVPAPAVKKISQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFECDLRRYATHERKMLLDNHALYDKTKRVFLGAESEEEIFAHLGLDYIEPWERNA

SEQ ID NO: 61 - protein, 391 aa, synthetic construct
MGGRDIDDGSEFSPSPVPGSQNVPAPAVKKISQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLCRYATHERKMLLDNHALYDKTKRVFLGAESEEEIFAHLGLDYIEPWERNA

SEQ ID NO: 62 - protein, 391 aa, synthetic construct
MGGRDIDDGSEFSPSPVPGSQNVPAPAVKKIPQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLEAESEEEIFAHLGLDYIEPWERNA

SEQ ID NO: 63 - protein, 391 aa, synthetic construct
MGGRDIDDGSEFSPSPVPGSQNVPAPAVKKIPQYAVQRRTTLNNYNQLFTDALDILAENDEFRENEGSALAFCRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFEWDLRRYANQERKMLLDNHALYDKTKRVFLEAESEEEIFAHLGLDYIEPWERNA

SEQ ID NO: 64 - protein, 391 aa, synthetic construct
MGGRDIDDGSEFSPSPVPGSQNVPAPAVKKIPQYAVQRRTTLNNYNQLFTDALDILAENDCFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLEAESEEEIFAHLGLDYIEPWERNA

SEQ ID NO: 65 - protein, 391 aa, synthetic construct
MGGRDIDDGSEFSPSPVPGSQNVPAPAVKKIPQYAVQRRTTLNNYNQLFTDALDILAENDEFRENCGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLEAESEEEIFAHLGLDYIEPWERNA

SEQ ID NO: 66 - protein, 391 aa, synthetic construct
MGGRDIDDGSEFSPSPVPGSQNVPAPAVKKIPQYAVQRRTTLNNYNQLFTDALDILAENDEFRENEGSCLAFMRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLEAESEEEIFAHLGLDYIEPWERNA

SEQ ID NO: 67 - protein, 391 aa, synthetic construct
MGGRDIDDGSEFSPSPVPGSQNVPAPAVKKIPQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFCSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLEAESEEEIFAHLGLDYIEPWERNA

SEQ ID NO: 68 - protein, 391 aa, synthetic construct
MGGRDIDDGSEFSPSPVPGSQNVPAPAVKKIPQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGCKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLEAESEEEIFAHLGLDYIEPWERNA

SEQ ID NO: 69 - protein, 391 aa, synthetic construct
MGGRDIDDGSEFSPSPVPGSQNVPAPAVKKIPQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVCALDHFQKGFLILKLDHQRVHSEESGQQEGKGWKAIRVDLVMPPYDRRAFALLGWTGSTQFERDLRRYATHERKMLLDNHALYDKTKRVFLEAESEEEIFAHLGLDYIEPWERNA

SEQ ID NO: 70 - protein, 391 aa, synthetic construct
MGGRDIDDGSEFSPSPVPGSQNVPAPAVKKIPQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDCLDHFQKGFLILKLDHQRVHSEESGQQEGKGWKAIRVDLVMPPYDRRAFALLGWTGSTQFERDLRRYATHERKMLLDNHALYDKTKRVFLEAESEEEIFAHLGLDYIEPWERNA

SEQ ID NO: 71 - protein, 391 aa, synthetic construct
MGGRDIDDGSEFSPSPVPGSQNVPAPAVKKIPQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSCQFERDLRRYATHERKMLLDNHALYDKTKRVFLEAESEEEIFAHLGLDYIEPWERNA

SEQ ID NO: 72 - protein, 391 aa, synthetic construct
MGGRDIDDGSEFSPSPVPGSQNVPAPAVKKIPQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFECDLRRYATHERKMLLDNHALYDKTKRVFLEAESEEEIFAHLGLDYIEPWERNA

SEQ ID NO: 73 - protein, 391 aa, synthetic construct
MGGRDIDDGSEFSPSPVPGSQNVPAPAVKKIPQYAVQRRTTLNNYNQLFTDALDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKTLRFTEMQKAGFKYYEDLVSGVTRPEAEAVSTLVKEAVVTFLPDALVTMTGGFRRGKMTGHDVDFLITSPEATEDEEQQLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLCRYATHERKMLLDNHALYDKTKRVFLEAESEEEIFAHLGLDYIEPWERNA

SEQ ID NO: 74 - protein, 391 aa, synthetic construct
MGGRDIDDGSEFSPSPVPGSQNVPAPAVKKISQYAVQRRTTLNNYNQLFTDALDILACNDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 75 - protein, 391 aa, synthetic construct
MGGRDIDDGSEFSPSPVPGSQNVPAPAVKKISQYAVQRRTTLNNYNQLFTDALCILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 76 - protein, 391 aa, synthetic construct
MGGRDIDDGSEFSPSPVPGSQNVPAPAVKKISQYAVQRRTTLNNYNQLFTDALDILAENDECRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 77 - protein, 391 aa, synthetic construct
MGGRDIDDGSEFSPSPVPGSQNVPAPAVKKISQYAVQRRTTLNNYNQLFTDALDILAENDEFRENEGSACAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 78 - protein, 391 aa, synthetic construct
MGGRDIDDGSEFSPSPVPGSQNVPAPAVKKISQYAVQRRTTLNNYNQLFTDALDILAENDEFRENEGSALAFMRACSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 79 - protein, 391 aa, synthetic construct
MGGRDIDDGSEFSPSPVPGSQNVPAPAVKKISQYAVQRRTTLNNYNQLFTDALDILAENDEFRENEGSALAFMRASSVLKSLPFPITSMKDLEGIPSLGDKVKSIIEEIIEDGESSEVKAVLNDERYKSFCLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 80 - protein, 391 aa, synthetic construct
MGGRDIDDGSEFSPSPVPGSQNVPAPAVKKISQYAVQRRTTLNNYNQLFTDCLDILAENDEFRENEGSALAFMRASSVLKSLPFPITSMKDLEGIPSLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNA

SEQ ID NO: 81 - protein, 367 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQT

SEQ ID NO: 82 - protein, 25 aa, synthetic construct
NSSSNNNNNNNNNNLGIEGRISHMS

SEQ ID NO: 83 - protein, 24 aa, synthetic construct
MGHHHHHHHHHHSSGHIDDDDKHM

SEQ ID NO: 84 - protein, 8 aa, unidentified
  source 1..8: note = TdT polymerase sequence motif
  VARIANT 2: note = A or S
  VARIANT 6: note = K or R
  VARIANT 8: note = P or T
YXCQRXTX

SEQ ID NO: 85 - protein, 349 aa, synthetic construct
LNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 86 - protein, 41 aa, synthetic construct
MGGRDIDDGSEFSPSPVPGSQNVPAPAVKKISQYAVQRRTT

SEQ ID NO: 87 - protein, 6 aa, synthetic construct
YPRAGP

SEQ ID NO: 88 - protein, 353 aa, synthetic construct
MEQQEGKGWKAIRVDLVMPPYDRRAFALLGWTGSRQFNRDLRRYATHERKMLLDNHALYDRTKEVFLEAESEEEIFAHLGLDYIEPWERNAGLLNNYNQLFTDALDILAENDEFRGNEGSALAFQRASSVLKSLPFPITSMEDLEGIPSLGDEVKSIIEEIIEDGESSEVMAVLNDERYKSFKLFTSVFGVGLKTAKKWYRMGFRTLSEVQSDKTLRFTEMQKAGFLYYEDLVSCVNRAEAEAVSMLVKEAVVTFLPDALVTMTGGFRRGKMTGHDVDFLITSPEATEDEEQRLLHKVTDFWKQQGLLLYANILESTFEKFKLPSRKVDGLDHFQKGFLILKLDHGRVHSEES

SEQ ID NO: 89 - protein, 356 aa, synthetic construct
SLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNAYPRAGPLNNYNQLFTDALDILAENDEFRENEGSALAFCRASSVLKSLPFPITSMKDLEGIP

SEQ ID NO: 90 - protein, 352 aa, synthetic construct
SLGDKVKSIIEEIIEDGESSEVKAVLNDERYKSFKLFTSVFGVGLKTAKKWYRMGFRTLSEVQSDKSLRFTEMQKAGFKYYEDLVSGVTRPEAEAVSVLVKEAVVTFLPDALVTMTGGFRRGKMTGHDVDFLITSPEATEDEEQRLLHKVTDFWKQQGLLLYADIAESTFEKFKLPSRKVDALDHFQKGFLILKLDHQRVHSEESGQQEGKGWKAIRVDLVMPPYDRRAFALLGWTGSTQFERDLRRYATHERKMLLDNHALYDKTKRVFLGAESEEEIFAHLGLDYIEPWERNAGLLNNYNQLFTDALDILAENDEFRENEGSALAFCRASSVLKSLPFPITSMKDLEGIP

SEQ ID NO: 91 - protein, 355 aa, synthetic construct
  VARIANT 295..300: note = This region may comprise YPRAGP or GL
CLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNAXXXXXXLNNCNQIFTDAFDILAENCEFRENEDSCVTFMRAASVLKSLPFTIISMKDTEGIP

SEQ ID NO: 92 - protein, 376 aa, synthetic construct
  VARIANT 316..321: note = This region may comprise YPRAGP or GL
CLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKGKTVTISPLDGKVSKLQKALRVFLEAESEEEIFAHLGLDYIEPWERNAXXXXXXLNNYNQLFTDALDILAENDELRENEGSCLAFMRASSVLKSLPFPITSMKDTEGIP

SEQ ID NO: 93 - protein, 355 aa, synthetic construct
  VARIANT 295..300: note = This region may comprise YPRAGP or GL
CLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNAXXXXXXLNNYNHIFTDAFEILAENSEFKENEVSYVTFMRAASVLKSLPFTIISMKDTEGIP

SEQ ID NO: 94 - protein, 328 aa, synthetic construct
  VARIANT 268..273: note = This region may comprise YPRAGP or GL
CMGDQVRDIIEEIIEEGESSRAKDVLNDERYKSFKQFTSVFGVGVKTSEKWYRMGLRTLEEVKAEKTLKLSKMQRVGLLYYEDLVSRVSEAEVEAVDLIVKKAVHMFLPDALVTVTGGFRRGKKIGHDIDFLITNPGPRDDDDLLHKGLLLYCDIIESTFVKEQRPSRHIDAMDNFQKCFAILKLYQPRADSRSYNTSEEFDTAEVKDWKAIRVDLVISPFEQYAYALLGWTGSKQFGRDLRRYANHEKKMMLDNHALYDKRKVLFIXXXXXXLNNLNKKFTDAFEIMAENYEFKENEIFSLEFQRAASVLKFLPFPVVRMKDIQGLP

SEQ ID NO: 95 - protein, 352 aa, synthetic construct
  VARIANT 292..297: note = This region may comprise YPRAGP or GL
CMGDQVRDIIEEIIEEGESSRAKDVLNDERYKSFKQFTSVFGVGVKTSEKWYRMGLRTLEEVKAEKTLKLSKMQRVGLLYYEDLVTCVSEAEANAVGLIVKKAVHTFLPDALVTLTGGFRRGKKIGHDIDFLITNPGPREDDDLLHKGLLLYCDIIESTFVKEQRPSRNVDAMDNFQKCFAILKLHQPPADNRSHSTSEEFDMAAVRDWKAIRVDLVISPFEQYAYALLGWTGSKQFGRDLRRYASHEKKMMLDNHALYDKRKRIFLKAGSEEEIFAHLGLDYVEPWERNAXXXXXXLNNFNKKFTDAFEIMAENYEFKENEIFSLEFLRAASVLKCLPFPVVRMRDVQGLP

SEQ ID NO: 96 - protein, 360 aa, synthetic construct
  VARIANT 300..305: note = This region may comprise YPRAGP or GL
CMGDQVRDIIEEIIEEGESSRVKDVLNDERYKSFKQFTSVFGVGVKTSEKWYRMGLRTLEEVTAEKTLKLSKMQKVGLLYYEDLVSCVSEAEADAVGLVVKKAVHTFLPDALVTLTGGFRRGKKIGHDIDFLITNPGPREDDDLLHKVVDLWKKQGLLLYCDIIESTFVKEQRPSRNIDAMDNFQKCFAILKLYQPRVDSESQSTSEEFDMAEVKDWKAIRVDLVISPFEQYAYALLGWTGSKQFGRDLRRYANHEKKMMLDNHALYDRRKRIFLKAGSEEEIFAHLGLDYVEPWERNAXXXXXXLNNFNKKFTDAFEIMAENYEFKENEIFSLEFLRAASVLKFLPFPVVRMKDIQGLP

SEQ ID NO: 97 - protein, 352 aa, synthetic construct
  VARIANT 292..297: note = This region may comprise YPRAGP or GL
CMGDQVRDIIEEIIEEGESSRAKDVLNDDRYKSFKQFTSVFGVGVKTSEKWYRMGLRTLEEVKAEKTLKLSKMQKVGLLYYEDLVSCVSEAEADAVGLVVKKAVHTFLPDALVTLTGGFRRGKKIGHDIDFLITNPGPREDDDLLHKGLLLYCDIIESTFVKEQRPSRNIDAMDNFQKCFAILKLYQPRVDSKSQSTSEELDMAEVKDWKAIRVDLVISPFEQYAYALLGWTGSKQFGRDLRRYASHEKKMMLDNHALYDRRKRIFLKAGSEEEIFAHLGLDYVEPWERNAXXXXXXLNNFNKKFTDAFEIMAENYEFKENEIFSLEFLRAASVMKFLPFPVVRMKDIQGLP

SEQ ID NO: 98 - protein, 352 aa, synthetic construct
  VARIANT 292..297: note = This region may comprise YPRAGP or GL
CMGDQVRDIIEEIIEEGESSRAKDVLNDDRYKSFKQFTSVFGVGVKTSEKWYRMGLRTLEEVKAEKTLKLSKMQKVGLLYYEDLVSCVSEAEADAVGLVVKKAVHTFLPDALVTLTGGFRRGKKIGHDIDFLITNPGPREDDDLLHKGLLLYCDIIESTFVKEQRPSRNIDAMDNFQKCFAILKLYQPRVDSKSQSTSEELDMAEVKDWKAIRVDLVISPFEQYAYALLGWTGSKQFGRDLRRYASHEKMMMLDNHALYDRRKRIFLKAGSEEEIFAHLGLDYVEPWERNAXXXXXXLNNLNKKFTDAFEIMAENYEFKENEIFSLEFLRAASVMKFLPFPVVRMKDIQGLP

SEQ ID NO: 99 - protein, 352 aa, synthetic construct
  VARIANT 292..297: note = This region may comprise YPRAGP or GL
CMGDQVRDIIEEIIEEGESSRAKDVLNDERYKSFKQFTSVFGVGVKTSEKWYRMGLRTLEEVKAEKTLKLSKMQRVGLLYYEDLVSCVSEAEVDAVGLIVKKTVHMFLPDALVTVTGGFRRGKKIGHDIDFLITNPGPREDDDLLHKGLLLYCDIIESTFVKEQRPSRNIDAMDNFQKCFAILKLYQPQVDNRSHNTSEEFNTAEVKGWKAIRVDLVISPFEQYAYALLGWTGSKQFGRDLRRYANHEKKMMLDNHALYDRRKRIFLKAGSEEEIFAHLGLDYLEPWERNAXXXXXXLNNFNKKFTDAFEIMAENYEFKENEIFSLEFQRAASVLKFLPFPVVRMKDIQGLP

SEQ ID NO: 100 - protein, 352 aa, synthetic construct
  VARIANT 292..297: note = This region may comprise YPRAGP or GL
CMGDHVRDIIEEIIEEGESSRAKDVLNDERYKSFKQFTSVFGVGVKTSEKWYRMGLRTLEEVKAEKTLKLSKMQRVGLLYYEDLVSCVSEAEVDAVGLIVKKTVHMFLPDALVTVTGGFRRGKKIGHDIDFLITNPGPREDDDLLHKGLLLYCDIIESTFVKEQRPSRNIDAMDNFQKCFAILKLYQPRVDNRSHNTSEEFKAAEVKDWKAIRVDLVISPFEQYPYALLGWTGSKQFGRDLRRYANHEKKMMLDNHALYDRRKRIFLKAGSEEEIFAHLGLDYLEPWERNAXXXXXXLSNFNKKFTDAFEIMAENYEFKENEVFSLEFQRAASVLKSLSFPVVRMKDIQGLP

SEQ ID NO: 101 - protein, 352 aa, synthetic construct
  VARIANT 292..297: note = This region may comprise YPRAGP or GL
CMGDQVKDIIEEIIEEGESSRTKDVLNDERYKSFKQFTSVFGVGVKTSEKWYRMGLRTLEEVKAEKTLKLSKMQRVGLLYYEDLVSCVSEAEVDAVGLIVKKAVHTFLPDALVTVTGGFRRGKKIGHDIDFLITNPGPRDDDDLLHKGLLLYCDIIESTFVKEQQPSRNIDAMDNFQKCFAILKLYQPQVDNRSYNTSEECDTAEVKDWKAIRVDLVISPFEQYAYALLGWTGSKQFGRDLRRYAHHEKKMMLDNHALYDRRKRIFLKAGSEEEIFAHLGLDYVEPWERNAXXXXXXLNNMNKKFTDAFEIMAENYEFKENEILSLEFQRAASVLKFLPFPVVRMKDIQGLP

SEQ ID NO: 102 - protein, 352 aa, synthetic construct
  VARIANT 292..297: note = This region may comprise YPRAGP or GL
CMGDQVRHIIEEIIEEGESSRAKDVLNDERYKSFKQFTSVFGVGVKTSEKWYRMGLRTLEEVKAEKTLKLSKMQRVGLLYYEDLVSCVSEAEVDAVGLIVKKAVHTFLPDALVTVTGGFRRGKKIGHDIDFLITNPGPREDDDLLHKGLLLYCDIIESTFVKEQQPSRNIDAMDNFQKCFAILKLYQPQVDNRSYNASEECDTAEVKDWKAIRVDLVISPFEQYAYALLGWTGSKQFGRDLRRYARHEKKMMLDNHALYDSRRKIFLKAGSEEEIFAHLGLDYVEPWERNAXXXXXXLNNLNKKFTDAFEIMAENYEFKENEILSLEFQRAASVLKFLPFPVLRMKDIQGLP

SEQ ID NO: 103 - protein, 352 aa, synthetic construct
  VARIANT 292..297: note = This region may comprise YPRAGP or GL
CIGDQVRDIIEEIIEEGESSRVKEVLNDERYKAFKQFTSIFGVGVKTSEKWYRMGLRTVEELKADKTLKLSKMQKAGFLYYEDLVSCVSKAEADAVTLIVKNTVCTFLPDALVTITGGFRRGKKMGHDIDFLITNPGPREDDELLHKGLLLYCDIIESTFVKEQLPSRKVDAMDHFQKCFTILKLYQPRVDNSSYDMSKKLDMAEVKDWKAIRVDLVITPFEQYAYALLGWTGSKQFGRDLRRYATHEKKMILDNHALYDRKKRIFLKAGSEEEIFAHLGLDYVEPWERNAXXXXXXLNNYNKKFTDAFEIMAENFEFKENEIFSLEFLRAASLLKSLPFPVTRMKDIQGLP

SEQ ID NO: 104 - protein, 355 aa, synthetic construct
  VARIANT 295..300: note = This region may comprise YPRAGP or GL
FLGKHSIAVIEEILQCGFSMEVQKILSNERFKTLKLFMSVFGVGLKTAEKWYRKGLLSFSDILAAPGIHLNRMQQNGFLHYGDISRAVSKAEAQALGNIIDETVHAITSDALLTLTGGFRRGKDFGHDVDFIVTTPEMGKEEHLLTAVIESLKHQGLILYCDYRASTFDMSRLPNHKFDAMDHFAKCFLILRLDGSRVEGGLCGAEGDGRGWRAVRVDLVSPPMKQYAFTLLGWTGSKQFERDLRRFAQKERHMLLDNHALYDKTKRELLAATTEQDIFAHLGLQYIEPCQRNAXXXXXXTANSNKIFTDAFEVLAESYEFNEMEGQSLAFRRAASVLRSLPSKIQSLRDAESLP

SEQ ID NO: 105 - protein, 407 aa, synthetic construct
  VARIANT 347..352: note = This region may comprise YPRAGP or GL
LKHHNSFLTEALEILAENAEFSENEGRSVAFRRSASVLKALPHAVRTIDELKDLPCLGEHSLSVIKEILEDGTSREVESTRQSEQFQAMKALTGVFGVGVKTADRWVREGLRSPLDLVDTGQKLNHAQQAGVQYYKDLNTPVTRKEAMVISVIVEKAVKALLPEAIITLTGGFRRGKEVGHDVDFLITHPEEGAEEKLMPKIVNWLEDQGLLLYQKTTRNSYLESKDGPARPPSNMDRFERCFSIFKLEQSKIDSNAHAEASSWRAIRVDLVVSPYSQFAFATLGWTGSKLFERELRRWAGQEKGMSLSSHALYDSKKKCYLQASSEEEIFTHLGLEYIPPSERNAXXXXXXLKHHNSFLTEALEILAENAEFSENEGRSVAFRRSASVLKALPHAVRTIDELKDLP

SEQ ID NO: 106 - protein, 357 aa, synthetic construct
  VARIANT 297..302: note = This region may comprise YPRAGP or GL
WLGEHTKAVMEEILTFGRSFKVEEIRCDERYQALKLFTSVFGVGPKTAEKWYRRGLRSLKEILTEPNIQLNRMQRAGFLYYSDISKAVSKAEAEALGCIIEETVHWIAPDAVLALTGGFRRGKEYGHDVDFLLTVPEMGKEEGLLLHVIDRLRDQGILLYCDHQGSTFDVSKLPSCRFEDMDHFQKCFLILRLEEGQVEGERGLQRDPGDSRGWRAVRVDLVAPPVDRYAFALLGWTGSKQFGRDLRTFARKERQMLLDNHALYDKTKKLFLPATTEEDIFAHLGLEYVEPWQRNAXXXXXXTENHNKIFTDVMEELAESSEFNESKGPCLAFRQAASVLKSLPSAVRCLGAIQGLP

SEQ ID NO: 107 - protein, 357 aa, synthetic construct
  VARIANT 297..302: note = This region may comprise YPRAGP or GL
CLGEHTKAVMEEILTFGRSFKVEEIRCDERYQALKLFTSVFGVGPKTAEKWYRRGLRSLQEILTEPNIQLNRMQRAGFLYYSDISKAVSKAEAKALGCIIEETVHWIAPDAILALTGGFRRGKEYGHDVDFLLTMPEMGKEEGLLLHVIDRLKDQGILLYCEYQGSTFDVSKLPSCRFEDMDCFQKCFLILRLEQGQVAGERGLQRDPGDSRGWRAVRVDLVAPPVDRYAFALLGWTGSKQFGRDLRTFAQKERLMLLDNHALYDKTKKLCLLATTEEDIFAHLGLEYVEPWQRNAXXXXXXTENHNKIFTDVMEELAESSEFNESKGPCLAFRQAASVLKSLPSAVHCLEAIQGLP

SEQ ID NO: 108 - protein, 356 aa, synthetic construct
  VARIANT 296..301: note = This region may comprise YPRAGP or GL
CLENKVKCIIEEIIEDGESSEVKAVLNDERYQSFKLFTSVFGVGLKTSEKWFRMGFRTLSKIRSDETLKFTRMQKAGFLYYEDLVSCVTRAEAEAVSVLVKEAVWAFLPDAFVTMTGGFRRGKQIGHDVDFLITSPGTTEAEEEQLLPKVINLWERKELLLYCDLVESTFEKSKLPSRKVDALDHFQKCFLILKLHHQRVDSGLSRRQEGKTWKAIRVDLVMCPYEHRAFALLGWTGSKQFERDLRRYATHERKMILDNHALYDKTKRIFLKAESEEEIFAHLGLDYIEPWERNAXXXXXXLNNCNHIFTDAFEVLAENYEFRENEIFSVTFLRAASVLKSLPFTIISMKDTEGIP

SEQ ID NO: 109 - protein, 356 aa, synthetic construct
  VARIANT 296..301: note = This region may comprise YPRAGP or GL
CLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNAXXXXXXLNNHNHIFTDAFEILAENCEFRENEGSYVTYMRAASVLKSLPFSIISMKDTEGIP

SEQ ID NO: 110 - protein, 321 aa, synthetic construct
  VARIANT 261..266: note = This region may comprise YPRAGP or GL
CLGDNVKCVIEEIIEDGESSEVKNVLNDERYQSFKLFTSVFGVGLKTSERWFRMGFRTLSTIRSDKSLKFTRMQKAGFLYYEDLVSCVTRAEAEAVSVLVKEAVWAFLPDALVTMTGGFRSQGLLLYHDLVESTFEKLKLPSRNVDALDHFQKCFLILKLDHQRVDSDKSNQQEGKTWKAIRVDLVMCPYECRAFALLGWTGSPQFERDLRRYATHERKMRLDNHALYDKTKRIFLKAESEEEIFAHLGLDYIEPWERNAXXXXXXLNNCNHMFTDAFEILAENYEFRENEDSYLAFKRAASVLKSLPFTIISMRDTEGIP

SEQ ID NO: 111 - protein, 351 aa, synthetic construct
  VARIANT 300..305: note = This region may comprise YPRAGP or GL
LIGVEMKYIIEEILEEGQCCRVLEVLNSERYKAFKAFTSIFGVGLKTSEKWFRMGLRTLEDIKYKKDLKLTTMQKHGLLYYDDIADYVSKMEAEAVEHLMKNIIYKFVPDAIVTITGGFRRGKELGHDVDLLITCPRKGVEREILHNTLNVLKNQGQLLFYDIYESTFDDTKRPSRRVDALDHYQKSFLILKLSRKEVKAEKQRDDGWTRLEDLRDWKAVRVDLVITPHEQYAFALLGWTGSPQFERDLRRYATQEKKMMLDNHALFDKTKNVFLKARSEEDIFAHLGLDYVLPEERNAXXXXXXDAFDILAENFEFEINKSPLVAFRRASSVLKSLQSPIMSTKDLEGLP

SEQ ID NO: 112 - protein, 417 aa, synthetic construct
  VARIANT 357..362: note = This region may comprise YPRAGP or GL
LQDINRIFTDALDTLAENYEFWENKGPLVAFRRASSVLKSLQSPIVSIKDLRGLPLLGDEMKHIIEEILEEGKCWRVLEVVNSERYNAFKVFTSIFGVGLKTAEKWHRMGFRTLEDIKTKKDLKLTRMQKHGLLYYEDITSHVSKMEADAVEHLVKNIICEFVPDAIVTITGGFRRGKDVGHDVDLLITCPRRGAEKNILHNTINVLKNQGRLLFYDIIESTFDDTKLPSRRVDALDHYQKSFIILKLLKKEVKAQIPRDYGWTEPEDTYPRDWKAVRVDLVISPQEQYAFALLGWTGSPQFERDLRRYASQEKKMMLDNHALFDKTKHIFMRARTEQDIFDHLGLDYVEPGDRNAXXXXXXLQDINRIFTDALDTLAENYEFWENKGPLVAFRRASSVLKSLQSPIVSIKDLRGLP

SEQ ID NO: 113 - protein, 362 aa, synthetic construct
  VARIANT 302..307: note = This region may comprise YPRAGP or GL
LLGDEMKHIIEEILEEGKCWRVLEVVNSERYKAFKVFTSIFGVGLKTAEKWHRMGFRTLEDIKTKKDLKLTRMQKHGLLYYEDITSHVSKMEADAVEHLVKNIICEFVPDAIVTITGGFRRGKDVGHDVDLLITCPRRGAEKNILHNTINVLKNQGRLLFYDIIESTFDDTKLPSRRVDALDHYQKSFIILKLLKKEVKAQIPEDDGWTEPEDTYPRDWKAVRVDLVISPQEQYAFALLGWTGSPQFERDLRRYASQEKKMMLDNHALFDKTKHIFLRARTEQDIFDHLGLDYLEPGDRNAXXXXXXLQDINRIFTDALDTLAENYEFLENKGPLVAFRRASSVLKSLQSPIVSIKDLRGLP

SEQ ID NO: 114 - protein, 413 aa, synthetic construct
  VARIANT 353..358: note = This region may comprise YPRAGP or GL
CLGDKVKCIIEEIIEDGESSEVKAVLNDERYKSFKLFTSVFGVGLKTSERWFRMGFRSLSKIRSDKSLKFTHMQKAGRGHRTPKHNGKAWEMLRKSQARNAAGPLRPGSNRVSLCLEFPPGLCAGWEGGNEGGQAGSSRGHGSEPSPTDKPDPDGLAQAQRRKPTCTDAEARALVQRDHRAPPCKASKPRRLDWCSLVLLYPFSYPFSVDRTCEGLLLYCDLVESTFEKLKIPSRKVDALDHFQKCFLILKLDHQRVDSDKASQQDGKTWKAIRVDLVMCPYERRAFALLGWTGSPQFERDLRRFATHERKMVLDNHALYDKTKRIFLKAESEEEIFAHLGLDYIEPWERNAXXXXXXLDNHNHVFTDAFEVLAENYEFRENEGCYLIFMRAASVLKSLPFTISSMKDIEGIP

SEQ ID NO: 115 - protein, 358 aa, synthetic construct
  VARIANT 298..303: note = This region may comprise YPRAGP or GL
HLGEEMKYIIEEILEDGKSSRVDQVIYSERYKSFKIFTSIFGVGLKTAEKWYRMGYRTLEDITSNKGLTFTSMQKHGLLYYEDITSSVTKTEADAVEHLIRTVICGFVSDAIVTVTGGFRRGKDVGHDVDILITCPQKRDENQILHKTINALKKRDLLLFCYLVESTFDDTKRPSRYIDSLDHFQKCFLILQLQKEESKNAYSHKPKGCDAKRSWKAIRVDLVVTPYEQYAFALLGWTGSQQFERDLRRYASHEKKMMLDNHAMYDKSKNIFLKAETEEDIFTHLGLEYLEPWERNAXXXXXXLQDINRIFTDAFDILAENYEFLENKGPRVAYQRASAVLKSLPFPILTMKDLEGLP

SEQ ID NO: 116 - protein, 350 aa, synthetic construct
  VARIANT 290..295: note = This region may comprise YPRAGP or GL
CLGEHSRAVIEEIIEYGSSSRVEEILNDERYRTMKLFSSVFGVGPKTSESWFCRGLRSFEQVLNEPSVRLNRMQTAGFLFYEDISKPVSRTEASALKIIVEEAVISVNPSATVSITGGFRRGKEFGHDVDFIIKTPEAGQEDAILTAVIERFKSQNILLYSDFQESTFDLRQLPTHCFEAMDHYSKCFLIIKLKREQETDARAGRDWRAVRVDLVAPPLERFAYALLGWTGSTLFERDLRRFARLERGKLLDNHTLYDKATNTFLPANTEEDIFAHLGLEYIEPWQRNAXXXXXXMDNHNKIFTDALEVLAENLEFGGNQGPSLAFRRAASVLKSLPAALKHLQETQHLP

SEQ ID NO: 117 - protein, 351 aa, synthetic construct
  VARIANT 291..296: note = This region may comprise YPRAGP or GL
CLGEHSKAVLEEIFECGSSSRAEDTLNDERYRTMKLFSSVFGVGPRTAESWYCRGLRTFEQVLTEPSIRLNRMQTAGFLFYEDISKPVSRTEASALKIIVEEAVMCVNPHATVHITGGFRRGKEFGHDVDFIIKTPEPGQENAVLPALIQRFKAQNILLYSDFQDSTFDLSQLPTHRFEAMDHFSKCFLIVKLKREQESGEREASRDWRAVRVDLVAPPLERYAYALLGWTGSTLFERDLRRFARLERGKLLDNHALYDKATKAFLPACTEEEIFAHLGLEYIEPWQRNAXXXXXXLDNHNKIFTDALEVLAENMEFVGNRGPCLAFRRAASVLKSLRAPLQHLEETQRLP

SEQ ID NO: 118 - protein, 355 aa, synthetic construct
  VARIANT 295..300: note = This region may comprise YPRAGP or GL
CLGEQIKSVIEEIFEFRTCSKVEEILRDEKYQTLKLFTSVFGVGPKTAEKWYRKGLRTLKAVLAEPDIHLNKMQEAGFLYYEDISKSVSKADAQAVETLIKDTISYYSSDTKLFLTGGFRRGKEHGHDVDFILTTPEIGKEQALLLFLIDKFKNLGILLYSDFQESTFDIQKLPSCKFEAMDHFPKCFLILKLKTGEQTAGVQQNQNDSRDWKAIRVDLVAPPADRYAFAMLGWTGSTQFERDLRRYARKERRMLLDNHALYDKTKHAFLEAQTEEEIFDLLGLEYIEPWQRNAXXXXXXLDNQNKIFTDAFEVLAESNEFLDSRGQCLAFRRAASVLKSLQFTLRSLDDIEGLP

SEQ ID NO: 119 - protein, 361 aa, synthetic construct
  VARIANT 301..306: note = This region may comprise YPRAGP or GL
WMGKQVKGIIEDIIEEGESSKVKAVLDNENYRSVKLFTSVFGVGLKTSDKWYRMGLRTLEEVKRDKNLKLTRMQKAGFLHYDDLTSCVSKAEADAASLIVQDVVWKIVPNAIVTIAGGFRRGKQTGHDVDFLITVPGSKQEEEELLHTVIDIWKKQELLLYYDLIESTFEDTKLPSRKVDALDHFQKCFAILKVHKEREDKGNSIRSKAFSEEEIKDWKAIRVDLVVVPFEQYAFALLGWTGSTQFERDLRRYATHEKKMMLDNHALYDKTKKIFLNAASEEEIFAHLGLDYLEPWERNAXXXXXXLKNYNQKFTDAFEILAENYEFNENKGFCTAFRRAASVLKCLPFTIVQVHDIEGVP

SEQ ID NO: 120 - protein, 346 aa, synthetic construct
  VARIANT 286..291: note = This region may comprise YPRAGP or GL
CLGDNSRAVLEEICECGSSSRVEEIQNDQRYRTMKLFCSVFGVGPKTAESWFCRGLKTFEQVLTEPSIRLNRMQTAGFLFYEDISVPVSRAEAAALKMMMEEALLFINPSATVTITGGFRRGKEFGHDVDFIIKAPEGQEDRILPAVIKRFKSQNVLLYSDFQKSTFDLRQLPNHRFEAMDRFSKCFLLVKLQMQESRTGRNWRAVRVDLVAPPLERFPYALLGWTGSTLFERDLRRFARLERGKLLDNHTLYDKTTKTFLPANTEEDIFQHLGLEYIEPWQRNAXXXXXXLENHNKIFTEALEVLALNSEFSGNQGSCLGFRRAASVLKSLPAALRSPEEALRLP

SEQ ID NO: 121 - protein, 4 aa, synthetic construct
TGSK

SEQ ID NO: 122 - protein, 4 aa, synthetic construct
TGSP

SEQ ID NO: 123 - protein, 4 aa, synthetic construct
TGSQ

SEQ ID NO: 124 - protein, 4 aa, synthetic construct
TGST

SEQ ID NO: 125 - protein, 4 aa, synthetic construct
TGSR

SEQ ID NO: 126 - protein, 362 aa, synthetic construct
IVDGSEFSPSPVPGSQNVPAPAVKKISQYACQRRTTLNNYNQLFTDALDILAENDELRENEGSALAFMRASSVLKSLPFPITSMKDTEGIPSLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYADILESTFEKFKQPSRKVDALDHFQKCFLILKLDHGRVHSEKSGQQEGKGWKAIRVDLVMSPYDRRAFALLGWTGSRQFERDLRRYATHERKMMLDNHALYDRTKRVFL

SEQ ID NO: 127 - protein, 357 aa, Tinamus guttatus
LMEQDEFPAVNTCEPEVPSFTVSKVSQYSCQRKTTLNNLNKKFTDAFEIMAENYEFKENEIFSLEFQRAASVLKFLPFPVVRMKDIQGLPCMGDQVRDIIEEIIEEGESSRAKDVLNDERYKSFKQFTSVFGVGVKTSEKWYRMGLRTLEEVKAEKTLKLSKMQRVGLLYYEDLVSRVSEAEVEAVDLIVKKAVHMFLPDALVTVTGGFRRGKKIGHDIDFLITNPGPRDDDDLLHKGLLLYCDIIESTFVKEQRPSRHIDAMDNFQKCFAILKLYQPRADSRSYNTSEEFDTAEVKDWKAIRVDLVISPFEQYAYALLGWTGSKQFGRDLRRYANHEKKMMLDNHALYDKRKVLFI

(19) United States
(12) Patent Application Publication
(10) Pub. No.: US 2025/0207113 A1
(43) Pub. Date: Jun. 26, 2025

(54) MODIFIED TERMINAL DEOXYNUCLEOTIDYL TRANSFERASE POLYMERASES

(71) Applicant: Ansa Biotechnologies, Inc., Emeryville, CA (US)

(72) Inventors: Jared Ellefson, El Cerrito, CA (US); Sebastian Barthel, Marburg (DE); Sebastian Palluk, Oakland, CA (US); Daniel Arlow, San Francisco, CA (US); Eric Estrin, Oakland, CA (US)

(21) Appl. No.: 18/971,708

(22) Filed: Dec. 6, 2024

Related U.S. Application Data
(63) Continuation of application No. PCT/US2023/068148, filed on Jun. 8, 2023.
(60) Provisional application No. 63/427,705, filed on Nov. 23, 2022.
(60) Provisional application No. 63/424,435, filed on Nov. 10, 2022.
(60) Provisional application No. 63/354,114, filed on Jun. 21, 2022.
(60) Provisional application No. 63/350,371, filed on Jun. 8, 2022.

(51) Int. Cl. C12N 9/12; C12P 19/34

(52) U.S. Cl. CPC C12N 9/1264; C12P 19/34; C12Y 207/07031

(57) ABSTRACT

The present disclosure provides modified terminal deoxynucleotidyl (TdT) polymerases and compositions (e.g., conjugates) comprising modified TdT polymerases. The present disclosure also provides circularly permuted terminal deoxynucleotidyl (TdT) polymerases as well as modified TdT polymerases and compositions (e.g., conjugates) comprising such cpTdT or modified TdT polymerases. Also provided are nucleic acids, vectors, and host cells encoding modified TdT polymerases, as well as methods including methods of producing modified TdT polymerases, and methods of using modified TdT polymerases (e.g., methods of nucleic acid synthesis).

### CROSS-REFERENCE TO APPLICATIONS

This application is a continuation of PCT/US2023/068148, filed Jun. 8, 2023, which claims priority to and the benefit of each of U.S. Provisional Application No. 63/350,371, filed Jun. 8, 2022, U.S. Provisional Application No. 63/424,435, filed Nov. 10, 2022, U.S. Provisional Application No. 63/427,705, filed Nov. 23, 2022, and U.S. Provisional Application No. 63/354,114, filed Jun. 21, 2022, the entire contents of each of which are incorporated by reference herein for all purposes.

### STATEMENT REGARDING FEDERALLY FUNDED RESEARCH

Some aspects of the present disclosure were made with government support under NSF Grant No. IIP-2036532 awarded by the National Science Foundation, and the government shares rights to such aspects of the present disclosure.

### REFERENCE TO A SEQUENCE LISTING

The instant application contains a Sequence Listing (XML file named ABB-010WO Sequence Listing.xml, generated on Jul. 14, 2023 and 171,260 bytes in size), which has been submitted electronically and is incorporated by reference herein.

### BACKGROUND

Terminal deoxynucleotidyl transferase (TdT) is a nucleotide polymerase capable of incorporating a nucleotide onto the 3′-OH of a polynucleotide in a template-independent manner. TdT can be used in a range of target applications, such as sequencing by synthesis and production of target polynucleotide sequences by enzymatic polynucleotide synthesis. Wild-type TdT has limited thermostability and nucleotide incorporation efficiency when used to incorporate nucleotide analogs. Accordingly, there is a need for improved TdT polymerases for use in a variety of applications.

### SUMMARY OF THE DISCLOSURE

In one aspect, the disclosure provides modified terminal deoxynucleotidyl transferases (TdTs). In some embodiments, a modified terminal deoxynucleotidyl transferase (TdT) polymerase comprises a sequence: (i) having at least 90% identity to that of the TdT polymerase of SEQ ID NO: 1; and (ii) having one or more amino acid substitutions relative to the amino acid sequence of the TdT polymerase of SEQ ID NO: 1. In some embodiments, the one or more amino acid substitutions is selected from the group consisting of A52C, D54E, D54C, D60A, L62F, L62C, S68R, L70C, S76A, S76C, T92L, I95L, S104R, G108E, I110L, K131C, F148Y, F166L, D206G, M211L, T235G, C36V, E64G, A118V, K157E, Q241R, C285A, E338N, I158V, P187S, D260N, S319P, M73Q, E145K, S163T, Q168E, P187A, Q271L, C285G, G294Q, M352L, L262A, R335T, V7D, L175K, N185T, M194V, K300E, R361K, E368G, E58C, C183G, M73C, R217C, K90E, K101E, K119M, R364E, D280A, D280N, D354A, D354L, H356V, H356A, L141C, D277C, A278C, R335C, R339C, R342C, A69C, T134C, E61C, S32P, M194T, E368K, R339W, T346N, H347Q, S163S, E66C, R361K, K141C, and combinations thereof.

In some embodiments, the one or more amino acid substitutions comprises C36V, G108E, A118V, and/or K157E. In some embodiments, the one or more amino acid substitutions further comprises V7D, L62F, T92L, E145K, F148Y, I158V, Q168E, L175K, C183G, N185T, Q271L, G294Q, K300E, S319P, and/or M352L. In some embodiments, the one or more amino acid substitutions further comprises C285G, L262A, R335T, and/or R361K.

In some embodiments, the one or more amino acid substitutions further comprises Q241R. In some embodiments, the one or more amino acid substitutions further comprises M194V and/or E368G. In some embodiments, the one or more amino acid substitutions further comprises S32P and M194T. In some embodiments, the one or more amino acid substitutions further comprises S163T. In some embodiments, the one or more amino acid substitutions further comprises E64G. In some embodiments, the one or more amino acid substitutions further comprises C285A. In some embodiments, the one or more amino acid substitutions further comprises E338N. In some embodiments, the one or more amino acid substitutions further comprises M73C. In some embodiments, the one or more amino acid substitutions further comprises D260N. In some embodiments, the one or more amino acid substitutions furthers comprises M73Q. In some embodiments, the one or more amino acid substitutions further comprises E58C.

In some embodiments, the one or more amino acid substitutions further comprises A69C, T134C, E61C, D277C, A278C, R339C, and/or R342C. In some embodiments, the one or more amino acid substitutions further comprises P187S. In some embodiments, the one or more amino acid substitutions further comprises P187A.

In some embodiments, the one or more amino acid substitutions further comprises (i) D280A or D280N, and/or (ii) D354L, and/or H356V. In some embodiments, the one or more amino acid substitutions further comprises R364E, K90E, K101E, and/or K119M. In some embodiments, the one or more amino acid substitutions further comprises R339W, T346N, and/or H347Q. In some embodiments, the one or more amino acid substitutions further comprises E66C and/or R361K.

In some embodiments, the one or more amino acid substitutions comprises R217C. In some embodiments, the one or more amino acid substitutions further comprises L141C. In some embodiments, the one or more amino acid substitutions further comprises R335C. In some embodiments, the one or more amino acid substitutions further comprises E368K. In some embodiments, the one or more amino acid substitutions further comprises S163S. In some embodiments, the one or more amino acid substitutions further comprises K141C.

In some embodiments, modified TdT polymerases of the present disclosure further comprise an amino acid sequence comprising at least one active site motif. In some embodiments, the active site motif corresponds to a homologous nucleotidyl transferase from a species selected from the group comprising Tinamus guttatus (SEQ ID NO: 5), Eudromia elegans (SEQ ID NO: 6), Nothoprocta perdicaria (SEQ ID NO: 7), Nothoprocta ornate (SEQ ID NO: 8), Nothoprocta pentlandii (SEQ ID NO: 9), Nothocercus Julius (SEQ ID NO: 10), Nothocercus nigrocapillus (SEQ ID NO: 11), Crypturellus undulatus (SEQ ID NO: 12), Crypturellus soui (SEQ ID NO: 13), Serilophus lunatus (SEQ ID NO: 14), Gouania willdenow (SEQ ID NO: 15), Silurus meridionalis (SEQ ID NO: 16), Coregonus clupeaformis (SEQ ID NO: 17), Oncorhynchus nerka (SEQ ID NO: 18), Vicugna pacos (SEQ ID NO: 19), Elephantulus edwardii (SEQ ID NO: 20), Galeopterus variegatus (SEQ ID NO: 21), Engystomops pustulosus (SEQ ID NO: 22), Bufo bufo (SEQ ID NO: 23), Bufo gargarizans (SEQ ID NO: 24), Sciurus carolinensis (SEQ ID NO: 25), Nanorana perkeri (SEQ ID NO: 26), Carassius auratus (SEQ ID NO: 27), Pimephales promelas (SEQ ID NO: 28), Chanos chanos (SEQ ID NO: 29), Python bivittatus (SEQ ID NO: 30), and Danio rerio (SEQ ID NO: 31), wherein the active site motif corresponds to positions 332-335 of the TdT polymerase of SEQ ID NO: 1. In some embodiments, the active site motif is selected from the group consisting of TGSK (SEQ ID NO: 121), TGSP (SEQ ID NO: 122), TGSQ (SEQ ID NO: 123), and TGST (SEQ ID NO: 124).

In one aspect, the disclosure provides modified TdT polymerases, which polymerases are chimeric TdT variant polymerases. In some embodiments, a chimeric TdT variant polymerase comprises a sequence having at least 90% identity to SEQ ID NO: 1, and an active site motif corresponding to positions 332-335 of SEQ ID NO: 1 comprising an active site motif identical to an active site motif from a homologous nucleotidyl transferase, wherein the homologous nucleotidyl transferase comprises a sequence selected from the group comprising SEQ ID NOs: 5-31. In some embodiments, the active site motif is selected from the group consisting of TGSK (SEQ ID NO: 121), TGSP (SEQ ID NO: 122), TGSQ (SEQ ID NO: 123), and TGST (SEQ ID NO: 124).

In some embodiments, a modified TdT polymerase as provided herein comprises a sequence having at least 80%, at least 85%, at least 90%, at least 95%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to an amino acid sequence selected from the group comprising SEQ ID NOs: 32-80. In some embodiments, the modified TdT polymerase is a chimeric variant polymerase.

In some embodiments, the modified TdT polymerases of the present disclosure comprise one or more improved properties compared to the TdT polymerase of SEQ ID NO: 1. In some embodiments, the one or more improved properties comprises increased thermostability, increased nucleotidyl transferase activity, and increased protease resistance.

In one aspect, the disclosure provides a modified TdT polymerase that is a circularly permuted polypeptide. In some such embodiments, the circularly permuted polypeptides comprise template-independent polymerase activity and an amino acid sequence derived from a parental polypeptide. In some such embodiments, the circularly permuted polypeptide has at least one functional difference as compared to the parental polypeptide. In some embodiments, the circularly permuted polypeptide is a terminal deoxynucleotidyl transferase (TdT) polymerase (cpTdT) and the parental polypeptide is a TdT polymerase. In some such embodiments, the cpTdTs have one or more parental TdTs. In some such embodiments, a parental TdT is or comprises a wild type TdT or components thereof. In some embodiments, a parental TdT is or comprises a modified TdT or components thereof.

A circularly permuted polypeptide comprising template-independent polymerase activity and comprising an amino acid sequence derived from a parental polypeptide, where the original N- and C-terminus are joined by a linker, and wherein a new N and C-terminus are formed by separating the parental sequence as described herein. In some embodiments, a parental TdT comprises or consists of a modified terminal deoxynucleotidyl transferase (TdT) polymerase as described herein. In some embodiments, the circularly permuted polypeptide has at least one functional difference as compared to the parental polypeptide.

In one aspect, the disclosure provides a polynucleotide comprising a sequence encoding a modified TdT polymerase as provided herein. In some embodiments, the sequence encoding a modified TdT polymerase is codon optimized.

In one aspect, the disclosure provides a vector comprising a polynucleotide comprising a sequence encoding a modified TdT polymerase as provided herein. In some embodiments, the sequence encoding the modified TdT polymerase is codon optimized.

In one aspect, the disclosure provides a host cell comprising a polynucleotide or a vector, wherein the polynucleotide or vector comprises a sequence encoding a modified TdT polymerase provided herein. In some embodiments, the sequence encoding the modified TdT polymerase is codon optimized.

In one aspect, the disclosure provides methods of producing modified TdT polymerases. In some embodiments, the method comprises (a) culturing a host cell provided herein under appropriate conditions for expressing a modified TdT polymerase of the present disclosure; and (b) isolating the modified TdT polymerase.

In one aspect, the disclosure provides conjugates comprising a modified TdT polymerase provided herein, a linker, and a nucleotide, wherein the linker tethers the nucleotide to the modified TdT polymerase. In some embodiments, the nucleotide is covalently linked to a TdT polymerase via the linker. In some embodiments, the linker is selectively cleavable.

In one aspect, the disclosure provides methods of nucleic acid synthesis. In some embodiments, a method comprises: a) providing a sample comprising a polynucleotide; b) providing a modified TdT polymerase provided herein and a nucleotide, or a conjugate provided herein; and c) contacting the sample comprising the polynucleotide with the modified TdT polymerase or the conjugate, wherein the modified TdT polymerase or the conjugate catalyzes an extension reaction comprising a covalent addition of the nucleotide (of the TdT polymerase/nucleotide or of the conjugate) onto the 3′ hydroxyl of said polynucleotide. In some embodiments, the method further comprises a cycle comprising repeating each of steps (a), (b), and (c) in order, and repeating the cycle one or more times to synthesize a nucleic acid. In some embodiments, the nucleic acid molecule is single stranded. In some embodiments, the nucleic acid molecule is double stranded.

In some aspects, the disclosure provides modified TdT polymerases as means to more efficiently catalyze addition of a nucleotide strand as compared to the TdT polymerase as set forth in SEQ ID NO: 1. In some embodiments, a modified TdT polymerase provided herein comprises one or more differences in properties compared to the TdT polymerase of SEQ ID NO: 1. In some embodiments, the one or more differences in properties is increased thermostability, increased nucleotidyl transferase activity, and/or increased protease resistance.

In some embodiments, a modified TdT polymerase provided herein comprises increased thermostability between a range of about 37° C. to about 55° C. In some embodiments, the increased thermostability comprises increased activity of a TdT polymerase provided herein at about 44° C., 48° C., 52° C., 54° C., and/or 55° C., wherein the increased activity is activity greater than about 75% of the modified TdT polymerase activity at 37° C.

In some embodiments, a modified TdT polymerase provided herein shows substantially no bias for incorporating a oligonucleotide substrate having a nucleotide terminal end of A, T, G, or C into a sequence.

In one aspect, the disclosure provides nucleotide extension reactions. In some embodiments, the nucleotide extension reaction uses a modified TdT polymerase provided herein, wherein the incorporation time of a nucleotide in a nucleotide extension reaction is less than about 60 seconds.

In one aspect, the disclosure provides circularly permuted polypeptides comprising template-independent polymerase activity.

In some embodiments, a circularly permuted polypeptide in accordance with the present disclosure comprises a sequence derived from one or more terminal deoxynucleotidyl transferase (TdT) sequences.

In some such embodiments, the circularly permuted polypeptide comprises a sequence having at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to two or more portions of a sequence of a TdT polymerase, wherein the sequence order of the portions are rearranged relative to the sequence of said TdT polymerase.

In some embodiments, the TdT is a wild type TdT. In some embodiments, the TdT is the modified TdT polymerase disclosed herein.

In some embodiments, a modified TdT polymerase as provided herein comprises an amino acid sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or more identity to that of the TdT polymerase of SEQ ID NO: 1. In some embodiments, a modified TdT polymerase disclosed herein comprises one or more amino acid substitutions relative to the amino acid sequence of the TdT polymerase of SEQ ID NO: 1.

In some such embodiments, the modified TdT polymerase comprises a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to an amino acid sequence selected from any one of SEQ ID NOs: 32-74.

In some embodiments, a circularly permuted polypeptide in accordance with the present disclosure comprises a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to SEQ ID NO: 88.

In some embodiments, a circularly permuted polypeptide in accordance with the present disclosure comprises a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to SEQ ID NO: 89.

In some embodiments, a circularly permuted polypeptide in accordance with the present disclosure comprises a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to SEQ ID NO: 90.

In some embodiments, a TdT in accordance with the present disclosure comprises an N-terminal truncation comprising removal of a region comprising an N-terminal amino acid through a defined sequence motif of a wild type TdT or a modified TdT polymerase, wherein the sequence motif is YX1CQRX2TX3, where X1 is A or S, X2 is K or R, and X3 is P or T (SEQ ID NO: 84).

In one aspect, the present disclosure provides polynucleotides, vectors, and/or host cells. In some embodiments, a polynucleotide comprises a sequence encoding a circularly permuted polypeptide. In some embodiments, a sequence encoding a circularly permuted polypeptide is codon-optimized. In some embodiments, a vector comprises one or more polynucleotides as provided herein. In some embodiments, a host cell comprises a polynucleotide disclosed herein or a vector disclosed herein.

In one aspect, the present disclosure provides methods of producing one or more circularly permuted polypeptides. In some such embodiments, the method comprises (a) culturing a host cell disclosed herein under appropriate conditions for expressing a circularly permuted polypeptide; and (b) isolating the circularly permuted polypeptide.

In one aspect, the disclosure provides a conjugate comprising: (a) a circularly permuted polypeptide disclosed herein; and (b) a nucleotide.

In some embodiments, a nucleotide is tethered to the circularly permuted polypeptide disclosed herein via a cleavable linker.

In one aspect, the disclosure provides methods of nucleic acid synthesis, comprising: (a) providing a circularly permuted polypeptide and a nucleotide as provided herein, or providing a conjugate as provided herein; (b) providing a sample comprising a polynucleotide; and (c) contacting the sample comprising a polynucleotide with the circularly permuted polypeptide and the nucleotide, or with the conjugate, wherein the circularly permuted polypeptide or the conjugate catalyzes an extension reaction comprising covalent addition of the nucleotide onto the 3′ hydroxyl of the polynucleotide.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale, with emphasis instead placed upon illustrating the principles of various embodiments of the disclosure.

FIG. 1 shows polymerase activity, as measured by level of fluorescence over time of an exemplary modified TdT polymerase at a range of temperatures.

FIG. 2 shows polymerase activity, as measured by level of fluorescence over time, of an exemplary modified TdT polymerase at a range of temperatures.

FIG. 3 shows polymerase activity, as measured by level of fluorescence over time, of an exemplary modified TdT polymerase at a range of temperatures.

FIGS. 4A-4D are heat map plots showing polymerase activity profiles of exemplary conjugates each comprising an exemplary modified TdT polymerases linked to a nucleotide (4A, dATP; 4B, dCTP; 4C, dGTP; and 4D, dTTP).

FIG. 5 is an alignment of an exemplary modified TdT polymerase amino acid sequence (“query”; SEQ ID NO: 126) and a non-Mus musculus nucleotidyl transferase amino acid sequence (“subject”; SEQ ID NO: 127), showing the active site motif in SEQ ID NO: 5 or 127 (underlined) corresponding to amino acid positions 332-335 of SEQ ID NO: 1 or 126.

FIGS. 6A-6C are exemplary electropherograms showing products (by fragment abundance (left y-axis) and percent of total products (right y-axis)) of an extension reaction catalyzed by an exemplary circularly permuted TdT conjugate and a dTTP after reaction times of 3.8 seconds (FIG. 6A), 11.6 seconds (FIG. 6B), and 45 seconds (FIG. 6C).

FIGS. 7A-7F are exemplary electropherograms showing products (by fragment abundance (left y-axis) and percent of total products (right y-axis)) of an extension reaction catalyzed by an exemplary TdT polymerase conjugate and a dTTP after reaction times of 3.8 seconds (FIG. 7A), 45 seconds (FIG. 7B), and 139 seconds (FIG. 7C) or the extension products of an extension reaction catalyzed by an exemplary circularly permuted TdT conjugate and a dTTP after reaction times of 3.8 seconds (FIG. 7D), 45 seconds (FIG. 7E), and 139 seconds (FIG. 7F).

### DETAILED DESCRIPTION

Details of various embodiments of the present disclosure are set forth herein. Among other things, the present disclosure provides technologies (e.g., compositions, methods, etc.) comprising modified TdT polymerases, including truncated TdT polymerases, TdT polymerases comprising one or more mutations, and circularly permuted TdT polymerases. Among other things, other features, objects, and advantages of technologies disclosed herein will be apparent from the description and the drawings, and from the claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “about” as used herein refers to the normal range of error for each value readily known to those skilled in the art. The term “about” value or reference to a parameter herein includes (and describes) an implementation of the value or parameter itself. For example, a description referring to “about X” includes a description of “X”. In some embodiments, “about” means a value of at most +/−10% of the recited value, e.g., +/−1%, +/−2%, +/−3%, +/−4%, +/−5%, +/−6% %, ±8%, ±9%, or ±10%.

### Terminal Deoxynucleotidyl Transferase

Terminal deoxynucleotidyl transferase is also known as “DNA Nucleotidylexotransferase,” “Deoxynucleotidyltransferase”, “TdT”, “terminal addition enzyme”, “terminal transferase”, “DNTT”, and “nucleotidylexotransferase”, “terminal deoxyribonucleotidyltransferase”, “deoxynucleotidyl terminal transferase”, “deoxyribonucleic acid nucleotidyltransferase”, “deoxyribonucleic nucleotidyltransferase”, and “terminal deoxynucleotide transferase.”

TdT is a member of the DNA polymerase type-X family and is a template-independent DNA polymerase that catalyzes the addition (i.e. incorporation) of a nucleotide to the 3′-hydroxyl terminus of oligonucleotide primers. TdT comprises activity described by IUBMB enzyme nomenclature EC 2.7.7.31. TdT cannot initiate a polynucleotide chain de novo.

As used herein, the term “nucleotide” refers to a molecule comprising a nucleoside and one or more phosphate groups. A “nucleoside” refers to a molecule comprising a nucleobase (e.g. adenine, thymine, cytosine, guanine, or uracil) and a five carbon sugar (e.g. ribose or 2′-deoxyribose). For example, in some embodiments, non-limiting examples of nucleotides include a nucleoside monophosphate, a nucleoside diphosphate, a nucleoside triphosphate, a nucleoside tetraphosphate, a nucleoside pentaphosphate, or a nucleoside hexaphosphate. TdT and modified TdT polymerases (e.g., TdT variants, chimeric TdT variants, circularly permuted TdTs) as provided herein, can incorporate any nucleoside polyphosphate, including nucleotide analogs comprising modifications to the nucleobase.

TdT is natively expressed in species spanning taxonomic ranks. For example, certain TdT homologs (sequences of which are set forth in Table 1) are expressed in Homo sapiens (NCBI Reference Sequence: NP_004079.3; SEQ ID NO: 2), Mus musculus (NCBI Reference Sequence: NP_033371.2; SEQ ID NO: 3), Bos taurus (NP_803461.1; SEQ ID NO: 4), Tinamus guttatus (GenBank: KGL73349.1; SEQ ID NO: 5), Eudromia elegans (GenBank: NXA34558.1; SEQ ID NO: 6), Nothoprocta perdicaria (NCBI Reference Sequence: XP_025906782.1; SEQ ID NO: 7), Nothoprocta ornate (GenBank: NWY00334.1; SEQ ID NO: 8), Nothoprocta pentlandii (GenBank: NWX83107.1; SEQ ID NO: 9), Nothocercus Julius (GenBank: NXA48721.1; SEQ ID NO: 10), Nothocercus nigrocapillus (GenBank: NXD14109.1; SEQ ID NO: 11), Crypturellus undulatus (GenBank: NWJ05551.1; SEQ ID NO: 12), Crypturellus soui (GenBank: NWI08830.1; SEQ ID NO: 13), Serilophus lunatus (GenBank: NXM66262.1; SEQ ID NO: 14), Gouania willdenow (NCBI Reference Sequence: XP_028324800.1; SEQ ID NO: 15), Silurus meridionalis (GenBank: KAF7701041.1; SEQ ID NO: 16), Coregonus clupeaformis (NCBI Reference Sequence: XP_041735872.1; SEQ ID NO: 17), Oncorhynchus nerka (NCBI Reference Number: XP_029496749.1; SEQ ID NO: 18), Vicugna pacos (XP_006210640.1; SEQ ID NO: 19), Elephantulus edwardii (NCBI Reference Sequence: XP_006880141.1; SEQ ID NO: 20), Galeopterus variegatus (NCBI Reference Sequence: XP_008573590.1; SEQ ID NO: 21), Engystomops pustulosus (GenBank: KAG8551716.1; SEQ ID NO: 22), Bufo bufo (NCBI Reference Sequence: XP_040294203.1; SEQ ID NO: 23), Bufo gargarizans (NCBI Reference Sequence: XP_044155657.1; SEQ ID NO: 24), Sciurus carolinensis (GenBank: MBZ3884855.1; SEQ ID NO: 25), Nanorana perkeri (NCBI Reference Sequence: XP_018414400.1; SEQ ID NO: 26), Carassius auratus (NCBI Reference Sequence: XP_026079168.1; SEQ ID NO: 27), Pimephales promelas (NCBI Reference Sequence: XP_039504943.1; SEQ ID NO: 28), Chanos chanos (NCBI Reference Sequence: XP_030628137.1; SEQ ID NO: 29), Python bivittatus (NCBI Reference Sequence: XP_007424457.1; SEQ ID NO: 30), and Danio rerio (GenBank: AAS89780.1; SEQ ID NO: 31).

In some embodiments, a TdTs of any one of SEQ ID NOs: 2-4 represents what is considered a wild-type TdT. In some embodiments, such a wild-type TdT may be used as a reference for which one or more changes may be made to arrive at, e.g., a modified TdT polymerase, e.g., as provided herein.

TABLE 1
Exemplary TdT Homologs and Sequences

| SEQ ID NO: | Sequence | Species |
|---|---|---|
| 2 | MDPPRASHLSPRKKRPRQTGALMASSPQDIKFQDLVVFILEKKMG | Homo sapiens |
| | TTRRAFLMELARRKGFRVENELSDSVTHIVAENNSGSDVLEWLQA | |
| | QKVQVSSQPELLDVSWLIECIRAGKPVEMTGKHQLVVRRDYSDST | |
| | NPGPPKTPPIAVQKISQYACQRRTTLNNCNQIFTDAFDILAENCE | |
| | FRENEDSCVTFMRAASVLKSLPFTIISMKDTEGIPCLGSKVKGII | |
| | EEIIEDGESSEVKAVLNDERYQSFKLFTSVFGVGLKTSEKWERMG | |
| | FRTLSKVRSDKSLKFTRMQKAGFLYYEDLVSCVTRAEAEAVSVLV | |
| | KEAVWAFLPDAFVTMTGGFRRGKKMGHDVDFLITSPGSTEDEEQL | |
| | LQKVMNLWEKKGLLLYYDLVESTFEKLRLPSRKVDALDHFQKCFL | |
| | IFKLPRQRVDSDQSSWQEGKTWKAIRVDLVLCPYERRAFALLGWT | |
| | GSRQFERDLRRYATHERKMILDNHALYDKTKRIFLKAESEEEIFA | |
| | HLGLDYIEPWERNA | |
| 3 | MDPLQAVHLGPRKKRPRQLGTPVASTPYDIRFRDLVLFILEKKMG | Mus musculus |
| | TTRRAFLMELARRKGFRVENELSDSVTHIVAENNSGSDVLEWLQL | |
| | QNIKASSELELLDISWLIECMGAGKPVEMMGRHQLVVNRNSSPSP | |
| | VPGSQNVPAPAVKKISQYACQRRTTLNNYNQLFTDALDILAENDE | |
| | LRENEGSCLAFMRASSVLKSLPFPITSMKDTEGIPCLGDKVKSII | |
| | EGIIEDGESSEAKAVLNDERYKSFKLFTSVFGVGLKTAEKWFRMG | |
| | FRTLSKIQSDKSLRFTQMQKAGFLYYEDLVSCVNRPEAEAVSMLV | |
| | KEAVVTFLPDALVTMTGGFRRGKMTGHDVDFLITSPEATEDEEQQ | |
| | LLHKVTDFWKQQGLLLYCDILESTFEKFKQPSRKVDALDHFQKCF | |
| | LILKLDHGRVHSEKSGQQEGKGWKAIRVDLVMCPYDRRAFALLGW | |
| | TGSRQFERDLRRYATHERKMMLDNHALYDRTKGKTVTISPLDGKV | |
| | SKLQKALRVFLEAESEEEIFAHLGLDYIEPWERNA | |
| 4 | MAQQRQHQRLPMDPLCTASSGPRKKRPRQVGASMASPPHDIKFQN | Bos taurus |
| | LVLFILEKKMGTTRRNFLMELARRKGFRVENELSDSVTHIVAENN | |
| | SGSEVLEWLQVQNIRASSQLELLDVSWLIESMGAGKPVEITGKHQ | |
| | LVVRTDYSATPNPGFQKTPPLAVKKISQYACQRKTTLNNYNHIFT | |
| | DAFEILAENSEFKENEVSYVTFMRAASVLKSLPFTIISMKDTEGI | |
| | PCLGDKVKCIIEEIIEDGESSEVKAVLNDERYQSFKLFTSVFGVG | |
| | LKTSEKWFRMGFRSLSKIMSDKTLKFTKMQKAGFLYYEDLVSCVT | |
| | RAEAEAVGVLVKEAVWAFLPDAFVTMTGGFRRGKKIGHDVDFLIT | |
| | SPGSAEDEEQLLPKVINLWEKKGLLLYYDLVESTFEKFKLPSRQV | |
| | DTLDHFQKCFLILKLHHQRVDSSKSNQQEGKTWKAIRVDLVMCPY | |
| | ENRAFALLGWTGSRQFERDIRRYATHERKMMLDNHALYDKTKRVF | |
| | LKAESEEEIFAHLGLDYIEPWERNA | |
| 5 | MERIRAPAAASQRKRQREMQSPNAPCTYEIKFNKFVIFIMQRKMG | Tinamus |
| | KTRRAFLMELARRKGFRVESELSDSVTHIVAENNSYLEVLDWLRG | guttatus |
| | QAVRDSSRFELLDISWFTACMEAGRPIELEMKYRLMEQDEFPAVN | |
| | TCEPEVPSFTVSKVSQYSCQRKTTLNNLNKKFTDAFEIMAENYEF | |
| | KENEIFSLEFQRAASVLKFLPFPVVRMKDIQGLPCMGDQVRDIIE | |
| | EIIEEGESSRAKDVLNDERYKSFKQFTSVFGVGVKTSEKWYRMGL | |
| | RTLEEVKAEKTLKLSKMQRVGLLYYEDLVSRVSEAEVEAVDLIVK | |
| | KAVHMFLPDALVTVTGGFRRGKKIGHDIDFLITNPGPRDDDDLLH | |
| | KGLLLYCDIIESTFVKEQRPSRHIDAMDNFQKCFAILKLYQPRAD | |
| | SRSYNTSEEFDTAEVKDWKAIRVDLVISPFEQYAYALLGWTGSKQ | |
| | FGRDLRRYANHEKKMMLDNHALYDKRKVLFI | |
| 6 | MERIRAPAAASQKKRQREMQSPNPPCSYKIKENKLVIFIMQRKMG | Eduromia |
| | KTRRTFLMELARRKGFRVESELSDSVTHIVAENNSYPEVLDWLRG | elegans |
| | QAVRDSSRFELLDISWFTACMEAGRPVESDVKYRLMEQDDFPTVK | |
| | TCEPEAPSFAASKVSQYSCQRKTTLNNFNKKFTDAFEIMAENYEF | |
| | KENEIFSLEFLRAASVLKCLPFPVVRMRDVQGLPCMGDQVRDIIE | |
| | EIIEEGESSRAKDVLNDERYKSFKQFTSVFGVGVKTSEKWYRMGL | |
| | RTLEEVKAEKTLKLSKMQRVGLLYYEDLVTCVSEAEANAVGLIVK | |
| | KAVHTFLPDALVTLTGGFRRGKKIGHDIDFLITNPGPREDDDLLH | |
| | KGLLLYCDIIESTFVKEQRPSRNVDAMDNFQKCFAILKLHQPPAD | |
| | NRSHSTSEEFDMAAVRDWKAIRVDLVISPFEQYAYALLGWTGSKQ | |
| | FGRDLRRYASHEKKMMLDNHALYDKRKRIFLKAGSEEEIFAHLGL | |
| | DYVEPWERNA | |
| 7 | MERIRAPAVASRKKSQREMQSPKPPCSYEIKENKFVIFIVQRKMG | Nothoprocta |
| | KTRRAFLMELARRKGFRVESELSDSVTHVVAENNSYSEVLDWLRG | perdicaria |
| | QAVQESSRFELLDISWFTACMEAGRPVEAEMKYRLLEQDECPAVN | |
| | TCEPEVPSFTASRVSQYSCQRKTTLNNFNKKFTDAFEIMAENYEF | |
| | KENEIFSLEFLRAASVLKFLPFPVVRMKDIQGLPCMGDQVRDIIE | |
| | EIIEEGESSRVKDVLNDERYKSFKQFTSVFGVGVKTSEKWYRMGL | |
| | RTLEEVTAEKTLKLSKMQKVGLLYYEDLVSCVSEAEADAVGLVVK | |
| | KAVHTFLPDALVTLTGGFRRGKKIGHDIDFLITNPGPREDDDLLH | |
| | KVVDLWKKQGLLLYCDIIESTFVKEQRPSRNIDAMDNFQKCFAIL | |
| | KLYQPRVDSESQSTSEEFDMAEVKDWKAIRVDLVISPFEQYAYAL | |
| | LGWTGSKQFGRDLRRYANHEKKMMLDNHALYDRRKRIFLKAGSEE | |
| | EIFAHLGLDYVEPWERNA | |
| 8 | MERMRAPAVASRKKSQREMQSPKPPCSYEIKENKFVIFIVQRKMG | Nothoprocta |
| | KTRRAFLMELARRKGFRVESELSDSVTHIVAENNSYSEVSDWLRG | ornate |
| | QAVQESSRFELLDISWFTACMEAGRPVETEMKYRLLEQDECPSMN | |
| | TCEPEVPSFTASKVSQYSCQRKTTLNNFNKKFTDAFEIMAENYEF | |
| | KENEIFSLEFLRAASVMKFLPFPVVRMKDIQGLPCMGDQVRDIIE | |
| | EIIEEGESSRAKDVLNDDRYKSFKQFTSVFGVGVKTSEKWYRMGL | |
| | RTLEEVKAEKTLKLSKMQKVGLLYYEDLVSCVSEAEADAVGLVVK | |
| | KAVHTFLPDALVTLTGGFRRGKKIGHDIDFLITNPGPREDDDLLH | |
| | KGLLLYCDIIESTFVKEQRPSRNIDAMDNFQKCFAILKLYQPRVD | |
| | SKSQSTSEELDMAEVKDWKAIRVDLVISPFEQYAYALLGWTGSKQ | |
| | FGRDLRRYASHEKKMMLDNHALYDRRKRIFLKAGSEEEIFAHLGL | |
| | DYVEPWERNA | |
| 9 | MERMRAPAVASRKKSQREMQSPKPPCSYEIKENKFVIFIVQRKMG | Nothoprocta |
| | KTRRAFLMELARRKGFRVESELSDSVTHIVAENNSYSEVSDWLRG | pentlandii |
| | QAVQESSRFELLDISWFTACMEAGRPVETEMKYRLLEQDECPAVN | |
| | TCEPEVPSFTASKVSQYSCQRKTTLNNLNKKFTDAFEIMAENYEF | |
| | KENEIFSLEFLRAASVMKFLPFPVVRMKDIQGLPCMGDQVRDIIE | |
| | EIIEEGESSRAKDVLNDDRYKSFKQFTSVFGVGVKTSEKWYRMGL | |
| | RTLEEVKAEKTLKLSKMQKVGLLYYEDLVSCVSEAEADAVGLVVK | |
| | KAVHTFLPDALVTLTGGFRRGKKIGHDIDFLITNPGPREDDDLLH | |
| | KGLLLYCDIIESTFVKEQRPSRNIDAMDNFQKCFAILKLYQPRVD | |
| | SKSQSTSEELDMAEVKDWKAIRVDLVISPFEQYAYALLGWTGSKQ | |
| | FGRDLRRYASHEKMMMLDNHALYDRRKRIFLKAGSEEEIFAHLGL | |
| | DYVEPWERNA | |
| 10 | MERIRAPAVASQRKRQREMQSPNPSCSYKIKFDKFVIFILQRKMG | Nothocercus |
| | KTRRAFLMELARRKGFRVESELSDSVTHIVAENNSYLEVLDWLRG | julius |
| | QAVQDSSRFELLDISWFTACMEAGRPVETDMKYRLVEQDELSTVN | |
| | TREPEVPSFTASKVSQYSCQRKTTLNNFNKKFTDAFEIMAENYEF | |
| | KENEIFSLEFQRAASVLKFLPFPVVRMKDIQGLPCMGDQVRDIIE | |
| | EIIEEGESSRAKDVLNDERYKSFKQFTSVFGVGVKTSEKWYRMGL | |
| | RTLEEVKAEKTLKLSKMQRVGLLYYEDLVSCVSEAEVDAVGLIVK | |
| | KTVHMFLPDALVTVTGGFRRGKKIGHDIDFLITNPGPREDDDLLH | |
| | KGLLLYCDIIESTFVKEQRPSRNIDAMDNFQKCFAILKLYQPQVD | |
| | NRSHNTSEEFNTAEVKGWKAIRVDLVISPFEQYAYALLGWTGSKQ | |
| | FGRDLRRYANHEKKMMLDNHALYDRRKRIFLKAGSEEEIFAHLGL | |
| | DYLEPWERNA | |
| 11 | MERIRAPAVVSQRKRQREMQSPNPPCSYEIKFDEFVIFIMQRKMG | Nothocercus |
| | KTRRAFLMELARRKGFRVESELSDSVTHIVAENNSYLEVLDWLRG | nigrocapillus |
| | QAVQDSSRFELLDVSWFTACMEAGRPVETDMKYRLMEQDELSTVN | |
| | TCEPEVPSFTASKVSQYSCQRKTTLSNFNKKFTDAFEIMAENYEF | |
| | KENEVFSLEFQRAASVLKSLSFPVVRMKDIQGLPCMGDHVRDIIE | |
| | EIIEEGESSRAKDVLNDERYKSFKQFTSVFGVGVKTSEKWYRMGL | |
| | RTLEEVKAEKTLKLSKMQRVGLLYYEDLVSCVSEAEVDAVGLIVK | |
| | KTVHMFLPDALVTVTGGFRRGKKIGHDIDFLITNPGPREDDDLLH | |
| | KGLLLYCDIIESTFVKEQRPSRNIDAMDNFQKCFAILKLYQPRVD | |
| | NRSHNTSEEFKAAEVKDWKAIRVDLVISPFEQYPYALLGWTGSKQ | |
| | FGRDLRRYANHEKKMMLDNHALYDRRKRIFLKAGSEEEIFAHLGL | |
| | DYLEPWERNA | |
| 12 | MERIRAPAAASQRKRQREMQSPNTPCSYEIKFNTFVIFIMQRKMG | Crypturellus |
| | KTRRAFLMELARRKGFRVESELSDSVTHIVAENNSYLEVMDWLRG | undulatus |
| | QAVQDSSRFELLDISWFTACMEAGRPVESEVKYRLMDKDEFPTVN | |
| | TCEPEVPSFTASKVSQYSCQRKTTLNNMNKKFTDAFEIMAENYEF | |
| | KENEILSLEFQRAASVLKFLPFPVVRMKDIQGLPCMGDQVKDIIE | |
| | EIIEEGESSRTKDVLNDERYKSFKQFTSVFGVGVKTSEKWYRMGL | |
| | RTLEEVKAEKTLKLSKMQRVGLLYYEDLVSCVSEAEVDAVGLIVK | |
| | KAVHTFLPDALVTVTGGFRRGKKIGHDIDFLITNPGPRDDDDLLH | |
| | KGLLLYCDIIESTFVKEQQPSRNIDAMDNFQKCFAILKLYQPQVD | |
| | NRSYNTSEECDTAEVKDWKAIRVDLVISPFEQYAYALLGWTGSKQ | |
| | FGRDLRRYAHHEKKMMLDNHALYDRRKRIFLKAGSEEEIFAHLGL | |
| | DYVEPWERNA | |
| 13 | MERIRAPAAASQRKRQREMQSPNAPCSYEIKENKFVIFLMQRKMG | Crypturellus |
| | KTRRAFLMELARRKGFRVESELSDSVTHIVAENNSYLEVMDWLRG | soui |
| | QAVQDSSRFELLDISWFTACMEAGRPVESEMKYRLMEQDEFPAVN | |
| | MCEPEVPSFTVSKVSQYSCQRKTTLNNLNKKFTDAFEIMAENYEF | |
| | KENEILSLEFQRAASVLKFLPFPVLRMKDIQGLPCMGDQVRHIIE | |
| | EIIEEGESSRAKDVLNDERYKSFKQFTSVFGVGVKTSEKWYRMGL | |
| | RTLEEVKAEKTLKLSKMQRVGLLYYEDLVSCVSEAEVDAVGLIVK | |
| | KAVHTFLPDALVTVTGGFRRGKKIGHDIDFLITNPGPREDDDLLH | |
| | KGLLLYCDIIESTFVKEQQPSRNIDAMDNFQKCFAILKLYQPQVD | |
| | NRSYNASEECDTAEVKDWKAIRVDLVISPFEQYAYALLGWTGSKQ | |
| | FGRDLRRYARHEKKMMLDNHALYDSRRKIFLKAGSEEEIFAHLGL | |
| | DYVEPWERNA | |
| 14 | MDRIKAPAVISHRKRQKELNSPKLTCGYEIKFSKFVIFIMQRKMG | Serilophus |
| | TTRRMFLMELARRNGFRVESELSDSVTHIVAENNSYLEVLDWLKG | lunatus |
| | QAVGDISRFELLDISWLTACMEAGRPVDSEIKYRLMNQCQDPLNT | |
| | PESEVPSSIARKVSHYSCQRKTTLNNYNKKFTDAFEIMAENFEFK | |
| | ENEIFSLEFLRAASLLKSLPFPVTRMKDIQGLPCIGDQVRDIIEE | |
| | IIEEGESSRVKEVLNDERYKAFKQFTSIFGVGVKTSEKWYRMGLR | |
| | TVEELKADKTLKLSKMQKAGFLYYEDLVSCVSKAEADAVTLIVKN | |
| | TVCTFLPDALVTITGGFRRGKKMGHDIDFLITNPGPREDDELLHK | |
| | GLLLYCDIIESTFVKEQLPSRKVDAMDHFQKCFTILKLYQPRVDN | |
| | SSYDMSKKLDMAEVKDWKAIRVDLVITPFEQYAYALLGWTGSKQF | |
| | GRDLRRYATHEKKMILDNHALYDRKKRIFLKAGSEEEIFAHLGLD | |
| | YVEPWERNA | |
| 15 | MLHGAVGFHIRRRSKMEKVPVSMHKDAKFESVRLYLVERKMGSSR | Gouania |
| | RAFLTQLARSKGFIVEDILSDVVTHIVSEDCQASSLWTWLEGHGV | willdenowi |
| | KTLPMMQVLDITWFTDSMKQGKPVAVEPRHLIQGSLPMLPDATTS | |
| | TPVAKVSQYACQRRTTTANSNKIFTDAFEVLAESYEFNEMEGQSL | |
| | AFRRAASVLRSLPSKIQSLRDAESLPFLGKHSIAVIEEILQCGES | |
| | MEVQKILSNERFKTLKLFMSVFGVGLKTAEKWYRKGLLSFSDILA | |
| | APGIHLNRMQQNGFLHYGDISRAVSKAEAQALGNIIDETVHAITS | |
| | DALLTLTGGFRRGKDFGHDVDFIVTTPEMGKEEHLLTAVIESLKH | |
| | QGLILYCDYRASTFDMSRLPNHKFDAMDHFAKCFLILRLDGSRVE | |
| | GGLCGAEGDGRGWRAVRVDLVSPPMKQYAFTLLGWTGSKQFERDL | |
| | RRFAQKERHMLLDNHALYDKTKRELLAATTEQDIFAHLGLQYIEP | |
| | CQRNA | |
| 16 | MVPLKRRKVTTEQTATHDSKACKFPETVIFIVERKMGASRKSFLT | Silurus |
| | RLGRSKGFLIEESFSSSITHVVSENNTGDEVHIWIKKQQKEGKAS | meridionalis |
| | TNLNSVNLLDISWLTESLASGNPVPVQDRHKLKTDPKPSEVTPVI | |
| | RMKSYACQRRTPLKHHNSFLTEALEILAENAEFSENEGRSVAFRR | |
| | SASVLKALPHAVRTIDELKDLPCLGEHSLSVIKEILEDGTSREVE | |
| | STRQSEQFQAMKALTGVFGVGVKTADRWVREGLRSPLDLVDTGQK | |
| | LNHAQQAGVQYYKDLNTPVTRKEAMVISVIVEKAVKALLPEAIIT | |
| | LTGGFRRGKEVGHDVDFLITHPEEGAEEKLMPKIVNWLEDQGLLL | |
| | YQKTTRNSYLESKDGPARPPSNMDRFERCFSIFKLEQSKIDSNAH | |
| | AEASSWRAIRVDLVVSPYSQFAFATLGWTGSKLFERELRRWAGQE | |
| | KGMSLSSHALYDSKKKCYLQASSEEEIFTHLGLEYIPPSERNA | |
| 17 | MNHAGMLVRVKKRKRPVEAGARGQVEVKFKEVTLYLVERKMGSSR | Coregonus |
| | RNFLTSLARSKGFRVEDVLSDEVTHVVAEDNQAEGLLAWLRGHGL | clupeaformis |
| | RDISRLAVLDISWFTDSMREGRPVTVETRHRIQNTVTDCSPPTAV | |
| | ANVSQYACQRRTTTENHNKIFTDVMEELAESSEFNESKGPCLAFR | |
| | QAASVLKSLPSAVRCLGAIQGLPWLGEHTKAVMEEILTFGRSFKV | |
| | EEIRCDERYQALKLFTSVFGVGPKTAEKWYRRGLRSLKEILTEPN | |
| | IQLNRMQRAGFLYYSDISKAVSKAEAEALGCIIEETVHWIAPDAV | |
| | LALTGGFRRGKEYGHDVDFLLTVPEMGKEEGLLLHVIDRLRDQGI | |
| | LLYCDHQGSTFDVSKLPSCRFEDMDHFQKCFLILRLEEGQVEGER | |
| | GLQRDPGDSRGWRAVRVDLVAPPVDRYAFALLGWTGSKQFGRDLR | |
| | TFARKERQMLLDNHALYDKTKKLFLPATTEEDIFAHLGLEYVEPW | |
| | QRNA | |
| 18 | MNHAGMLALVKKRKRPVEAGAQGQVEVKFKEVTLYLVERKMGSSR | Oncorhynchus |
| | RNFLTSLARSKGFRVEDVLSDDVTHVVAEDNQAEVLWAWLMGHGL | nerka |
| | RDVSRVAVLDISWFTDSMREGRPVRVETRHCIQNTPTGTDCSPPT | |
| | AVANVSQYACQRRITTENHNKIFTDVMEELAESSEFNESKGPCLA | |
| | FRQAASVLKSLPSAVHCLEAIQGLPCLGEHTKAVMEEILTFGRSF | |
| | KVEEIRCDERYQALKLFTSVFGVGPKTAEKWYRRGLRSLQEILTE | |
| | PNIQLNRMQRAGFLYYSDISKAVSKAEAKALGCIIEETVHWIAPD | |
| | AILALTGGFRRGKEYGHDVDFLLTMPEMGKEEGLLLHVIDRLKDQ | |
| | GILLYCEYQGSTFDVSKLPSCRFEDMDCFQKCFLILRLEQGQVAG | |
| | ERGLQRDPGDSRGWRAVRVDLVAPPVDRYAFALLGWTGSKQFGRD | |
| | LRTFAQKERLMLLDNHALYDKTKKLCLLATTEEDIFAHLGLEYVE | |
| | PWQRNA | |
| 19 | MDPQQMAPSGPRKKRPRQVGASVASPPHDIKFQDLVLFILEKKMG | Vicugna pacos |
| | TTRRTFLMELARRKGFRVENELSDSVTHIVAENNSGSEVLEWLQV | |
| | QNIKAGSQLELLDVSWLIESMEAGKPVEMTGKHQLVVKTDCSGSP | |
| | NPGFQKTPPLAVKKISQYACQRRTTLNNCNHIFTDAFEVLAENYE | |
| | FRENEIFSVTFLRAASVLKSLPFTIISMKDTEGIPCLENKVKCII | |
| | EEIIEDGESSEVKAVLNDERYQSFKLFTSVFGVGLKTSEKWERMG | |
| | FRTLSKIRSDETLKFTRMQKAGFLYYEDLVSCVTRAEAEAVSVLV | |
| | KEAVWAFLPDAFVTMTGGFRRGKQIGHDVDFLITSPGTTEAEEEQ | |
| | LLPKVINLWERKELLLYCDLVESTFEKSKLPSRKVDALDHFQKCF | |
| | LILKLHHQRVDSGLSRRQEGKTWKAIRVDLVMCPYEHRAFALLGW | |
| | TGSKQFERDLRRYATHERKMILDNHALYDKTKRIFLKAESEEEIF | |
| | AHLGLDYIEPWERNA | |
| 20 | MDPLQMACTGPRKKRARQMDTSMGSTQDIKFQDLVLFILEKKMGT | Elephantulus |
| | TRRAFLMELARRKGFRVENELSDSVTHIVAENNSGSDVLEWLQVQ | edwardii |
| | KIKASSQLELLDVSWLIECMGAGKLVEITGKHQLVSIMVRGDCPA | |
| | SHDSSPQKTESAAVQKISQYACQRRTTLNNHNHIFTDAFEILAEN | |
| | CEFRENEGSYVTYMRAASVLKSLPFSIISMKDTEGIPCLADKVKC | |
| | VIEEIIEDGESSEVKAVLNDERYKSFKLFTSVFGVGLKTAEKWFR | |
| | LGFRTLSGIMNDKTLKLTHMQKAGFLYYEDLVSCVTRAEAEAVGV | |
| | LVKEAVWAFLPDAIVTMTGGFRRGKKVGHDVDFLITSPEATEEQE | |
| | QQLLHKVITFWEKEGLLLYCDLYESTFEKLKMPSRKVDALDHFQK | |
| | CFLILKLHRECVDDGTSSQLQGKTWKAIRVDLVVCPYECRAFALL | |
| | GWTGSPQFERDLRRYATHERKMMLDNHALYDKTKRKFLSADSEED | |
| | IFAHLGLDYIEPWERNA | |
| 21 | MDLLPMAHSGPRKKRPRQTGTSMASTSHDIKFQDLVLFILEKKMG | Galeopterus |
| | TTRRAFLMELARRKGFRVDNELSDSVTHIVAENNSGSDVLEWLQV | variegatus |
| | QKIRASSQLELLDVSWLIECMRAGKPVEMTGKHQLVVKRDYLASP | |
| | NPGPQKTLPLAAQKISPYACQRRTTLNNCNHMFTDAFEILAENYE | |
| | FRENEDSYLAFKRAASVLKSLPFTIISMRDTEGIPCLGDNVKCVI | |
| | EEIIEDGESSEVKNVLNDERYQSFKLFTSVFGVGLKTSERWERMG | |
| | FRTLSTIRSDKSLKFTRMQKAGFLYYEDLVSCVTRAEAEAVSVLV | |
| | KEAVWAFLPDALVTMTGGFRSQGLLLYHDLVESTFEKLKLPSRNV | |
| | DALDHFQKCFLILKLDHQRVDSDKSNQQEGKTWKAIRVDLVMCPY | |
| | ECRAFALLGWTGSPQFERDLRRYATHERKMRLDNHALYDKTKRIF | |
| | LKAESEEEIFAHLGLDYIEPWERNA | |
| 22 | DAFDILAENFEFEINKSPLVAFRRASSVLKSLQSPIMSTKDLEGL | Engystomops |
| | PLIGVEMKYIIEEILEEGQCCRVLEVLNSERYKAFKAFTSIFGVG | pustulosus |
| | LKTSEKWFRMGLRTLEDIKYKKDLKLTTMQKHGLLYYDDIADYVS | |
| | KMEAEAVEHLMKNIIYKFVPDAIVTITGGFRRGKELGHDVDLLIT | |
| | CPRKGVEREILHNTLNVLKNQGQLLFYDIYESTEDDTKRPSRRVD | |
| | ALDHYQKSFLILKLSRKEVKAEKQRDDGWTRLEDLRDWKAVRVDL | |
| | VITPHEQYAFALLGWTGSPQFERDLRRYATQEKKMMLDNHALFDK | |
| | TKNVFLKARSEEDIFAHLGLDYVLPEERNA | |
| 23 | MELVRTPLLVPIKKKPKLLHISSSFYHYEVKFKGIVLYLLERKMG | Bufo bufo |
| | ASRRNFLMELGRKRGFQIESELRDSVTHIVAENNSGDDVLEWLQT | |
| | TKLTKANPYQILDISWFTECMGAGYPINIECRHRLQVQQDCSASF | |
| | NPPLSSSCVHVSPYACQRRTPLQDINRIFTDALDTLAENYEFWEN | |
| | KGPLVAFRRASSVLKSLQSPIVSIKDLRGLPLLGDEMKHIIEEIL | |
| | EEGKCWRVLEVVNSERYNAFKVFTSIFGVGLKTAEKWHRMGFRTL | |
| | EDIKTKKDLKLTRMQKHGLLYYEDITSHVSKMEADAVEHLVKNII | |
| | CEFVPDAIVTITGGFRRGKDVGHDVDLLITCPRRGAEKNILHNTI | |
| | NVLKNQGRLLFYDIIESTFDDTKLPSRRVDALDHYQKSFIILKLL | |
| | KKEVKAQIPRDYGWTEPEDTYPRDWKAVRVDLVISPQEQYAFALL | |
| | GWTGSPQFERDLRRYASQEKKMMLDNHALFDKTKHIFMRARTEQD | |
| | IFDHLGLDYVEPGDRNA | |
| 24 | MELVRTPTLVPIKKKPKLLQVSSSFYHYEVKFKGIVLYLLERKMG | Bufo |
| | ASRRNFLMELGRKRGFQIESELRDSVTHIVAENNSGDEVLEWLQT | gargarizans |
| | TKLTKANLYQILDISWFTECMGAGYPIIIECRHRLQVQQDCSASF | |
| | NPPLSSSCVHVSPYACQRQTPLQDINRIFTDALDTLAENYEFLEN | |
| | KGPLVAFRRASSVLKSLQSPIVSIKDLRGLPLLGDEMKHIIEEIL | |
| | EEGKCWRVLEVVNSERYKAFKVFTSIFGVGLKTAEKWHRMGFRTL | |
| | EDIKTKKDLKLTRMQKHGLLYYEDITSHVSKMEADAVEHLVKNII | |
| | CEFVPDAIVTITGGFRRGKDVGHDVDLLITCPRRGAEKNILHNTI | |
| | NVLKNQGRLLFYDIIESTFDDTKLPSRRVDALDHYQKSFIILKLL | |
| | KKEVKAQIPEDDGWTEPEDTYPRDWKAVRVDLVISPQEQYAFALL | |
| | GWTGSPQFERDLRRYASQEKKMMLDNHALFDKTKHIFLRARTEQD | |
| | IFDHLGLDYLEPGDRNA | |
| 25 | MTLNWVLVLVLHQAIGLVITHRGSHTPAFRDAKDQARHVRITHEL | Sciurus |
| | EEVTQNLLPCTKQRNESQVGIATIGSSDSVTHIVAENNSGSDVLE | carolinensis |
| | WLQVQNIKASSQLELLDISWLIECMRAGRPVETTGKHQLVRRADS | |
| | AHSNPDPQRIPAHAVRKISQYACQRRTTLDNHNHVFTDAFEVLAE | |
| | NYEFRENEGCYLIFMRAASVLKSLPFTISSMKDIEGIPCLGDKVK | |
| | CIIEEIIEDGESSEVKAVLNDERYKSFKLFTSVFGVGLKTSERWF | |
| | RMGFRSLSKIRSDKSLKFTHMQKAGRGHRTPKHNGKAWEMLRKSQ | |
| | ARNAAGPLRPGSNRVSLCLEFPPGLCAGWEGGNEGGQAGSSRGHG | |
| | SEPSPTDKPDPDGLAQAQRRKPTCTDAEARALVQRDHRAPPCKAS | |
| | KPRRLDWCSLVLLYPFSYPFSVDRTCEGLLLYCDLVESTFEKLKI | |
| | PSRKVDALDHFQKCFLILKLDHQRVDSDKASQQDGKTWKAIRVDL | |
| | VMCPYERRAFALLGWTGSPQFERDLRRFATHERKMVLDNHALYDK | |
| | TKRIFLKAESEEEIFAHLGLDYIEPWERNA | |
| 26 | MDLVRGSGLIPVKKRPKILHGSSSFCHYEVKFKGIVLFLVERKMG | Nanorana |
| | TSRRNFLMELAKKRGFQIERELRDCVTHIVAENNSGAEVVEWLQS | parkeri |
| | RKLNNAVKCQIVDISWFTECMGAGCPIKIESRHQLTVQEDCPASF | |
| | NMPVSSSCVRVSPYACQRRTPLQDINRIFTDAFDILAENYEFLEN | |
| | KGPRVAYQRASAVLKSLPFPILTMKDLEGLPHLGEEMKYIIEEIL | |
| | EDGKSSRVDQVIYSERYKSFKIFTSIFGVGLKTAEKWYRMGYRTL | |
| | EDITSNKGLTFTSMQKHGLLYYEDITSSVTKTEADAVEHLIRTVI | |
| | CGFVSDAIVTVTGGFRRGKDVGHDVDILITCPQKRDENQILHKTI | |
| | NALKKRDLLLFCYLVESTFDDTKRPSRYIDSLDHFQKCFLILQLQ | |
| | KEESKNAYSHKPKGCDAKRSWKAIRVDLVVTPYEQYAFALLGWTG | |
| | SQQFERDLRRYASHEKKMMLDNHAMYDKSKNIFLKAETEEDIFTH | |
| | LGLEYLEPWERNA | |
| 27 | MFPKALPTRKRRRTEVASVGGREEVKFRDVTLFLVEKRMGKSRRN | Carassius |
| | FLSNLARSKGFCVDDALSADVTHVVSEGNAAQELWSWLEEQGFRE | auratus |
| | MQDKHVLHISWFTESMSAGRPVPPQIRHYIQNPAVDQRSRTNPSA | |
| | KTESALSPYACQRRTTMDNHNKIFTDALEVLAENLEFGGNQGPSL | |
| | AFRRAASVLKSLPAALKHLQETQHLPCLGEHSRAVIEEIIEYGSS | |
| | SRVEEILNDERYRTMKLFSSVFGVGPKTSESWFCRGLRSFEQVLN | |
| | EPSVRLNRMQTAGFLFYEDISKPVSRTEASALKIIVEEAVISVNP | |
| | SATVSITGGFRRGKEFGHDVDFIIKTPEAGQEDAILTAVIERFKS | |
| | QNILLYSDFQESTFDLRQLPTHCFEAMDHYSKCFLIIKLKREQET | |
| | DARAGRDWRAVRVDLVAPPLERFAYALLGWTGSTLFERDLRRFAR | |
| | LERGKLLDNHTLYDKATNTFLPANTEEDIFAHLGLEYIEPWQRNA | |
| 28 | MLPTRKRRPEASFPRGHDEVKFKDLTLYLVERRMGKSRRNFLSNL | Pimephales |
| | ARSKGFCVDDALSGNVTHVVSEGNAAQDLWHWLEEQGFGETPHTH | promelas |
| | VLDVSWFTESMSAGRPVLVELRHYIQNPAVDHRSCAHPSGKPESA | |
| | VSAYACQRRTTLDNHNKIFTDALEVLAENMEFVGNRGPCLAFRRA | |
| | ASVLKSLRAPLQHLEETQRLPCLGEHSKAVLEEIFECGSSSRAED | |
| | TLNDERYRTMKLFSSVFGVGPRTAESWYCRGLRTFEQVLTEPSIR | |
| | LNRMQTAGFLFYEDISKPVSRTEASALKIIVEEAVMCVNPHATVH | |
| | ITGGFRRGKEFGHDVDFIIKTPEPGQENAVLPALIQRFKAQNILL | |
| | YSDFQDSTFDLSQLPTHRFEAMDHFSKCFLIVKLKREQESGEREA | |
| | SRDWRAVRVDLVAPPLERYAYALLGWTGSTLFERDLRRFARLERG | |
| | KLLDNHALYDKATKAFLPACTEEEIFAHLGLEYIEPWQRNA | |
| 29 | MLQSPFLPPVRKRRRPEMTPARVQHEVRFEGVTLYLVERRMGSSR | Chanos chanos |
| | RNFLTHLARSKGFCVHDILSSEVTHIVAEGNPAEELWSWLEEHHL | |
| | RDMCNLHVLDIRWFTESMQAGRPVAVETRHYIQKPSSKLKTALPA | |
| | TSKPTVSQYACQRRTTLDNQNKIFTDAFEVLAESNEFLDSRGQCL | |
| | AFRRAASVLKSLQFTLRSLDDIEGLPCLGEQIKSVIEEIFEFRTC | |
| | SKVEEILRDEKYQTLKLFTSVFGVGPKTAEKWYRKGLRTLKAVLA | |
| | EPDIHLNKMQEAGFLYYEDISKSVSKADAQAVETLIKDTISYYSS | |
| | DTKLFLTGGFRRGKEHGHDVDFILTTPEIGKEQALLLFLIDKFKN | |
| | LGILLYSDFQESTFDIQKLPSCKFEAMDHFPKCFLILKLKTGEQT | |
| | AGVQQNQNDSRDWKAIRVDLVAPPADRYAFAMLGWTGSTQFERDL | |
| | RRYARKERRMLLDNHALYDKTKHAFLEAQTEEEIFDLLGLEYIEP | |
| | WQRNA | |
| 30 | MNKMKTSDFSPMRKRQKRMQIAAPLSSYKIEFKDIIIFIVERKMG | Python |
| | MTRRMFLMDLARRKGFRVENELSDLVTHVVAENNSCSEILKWLQK | bivittatus |
| | HNVEDSSRFRILDIRWLTACMEVGRPVDSEKYQLPEDEDRSVTSD | |
| | LDRDSISEYACQRRTTLKNYNQKFTDAFEILAENYEFNENKGFCT | |
| | AFRRAASVLKCLPFTIVQVHDIEGVPWMGKQVKGIIEDIIEEGES | |
| | SKVKAVLDNENYRSVKLFTSVFGVGLKTSDKWYRMGLRTLEEVKR | |
| | DKNLKLTRMQKAGFLHYDDLTSCVSKAEADAASLIVQDVVWKIVP | |
| | NAIVTIAGGFRRGKQTGHDVDFLITVPGSKQEEEELLHTVIDIWK | |
| | KQELLLYYDLIESTFEDTKLPSRKVDALDHFQKCFAILKVHKERE | |
| | DKGNSIRSKAFSEEEIKDWKAIRVDLVVVPFEQYAFALLGWTGST | |
| | QFERDLRRYATHEKKMMLDNHALYDKTKKIFLNAASEEEIFAHLG | |
| | LDYLEPWERNA | |
| 31 | MLPSRKRVRAEVSTSQGGEHVKFSDVTLFLVEKRMGKSRRTFLSS | Danio rerio |
| | LARSKGFCVDDALSGAVTHVVSEGLSAQDLWLWLEDQGFQETHSK | |
| | HVLNISWFTESMSAGRPLPVEDTHCIQNPAADQRSCVHLSATPES | |
| | AVSPYACQRRTTLENHNKIFTEALEVLALNSEFSGNQGSCLGFRR | |
| | AASVLKSLPAALRSPEEALRLPCLGDNSRAVLEEICECGSSSRVE | |
| | EIQNDQRYRTMKLFCSVFGVGPKTAESWFCRGLKTFEQVLTEPSI | |
| | RLNRMQTAGFLFYEDISVPVSRAEAAALKMMMEEALLFINPSATV | |
| | TITGGFRRGKEFGHDVDFIIKAPEGQEDRILPAVIKRFKSQNVLL | |
| | YSDFQKSTFDLRQLPNHRFEAMDRFSKCFLLVKLQMQESRTGRNW | |
| | RAVRVDLVAPPLERFPYALLGWTGSTLFERDLRRFARLERGKLLD | |
| | NHTLYDKTTKTFLPANTEEDIFQHLGLEYIEPWQRNA | |

### Modified TdT Polymerases

Provided herein, among other things, are engineered, modified terminal deoxynucleotidyl transferase (TdT) polymerases. As used herein, a “modified TdT polymerase” refers to a terminal deoxynucleotidyl transferase (TdT) polymerase having an amino acid sequence that is less than 100% identical to the amino acid sequence of a reference TdT polymerase as set forth in SEQ ID NO: 1. In some embodiments, the amino acids are synthetic and/or comprised of synthetic nucleotides.

As will be known to those of skill in the art, the TdT polymerase of SEQ ID NO: 1 has an amino acid sequence that differs from wild-type TdT polymerase (e.g. SEQ ID NO: 3). In some embodiments, such a modified TdT polymerase comprises one or more amino acid differences relative to a wild-type TdT polymerase and/or the TdT polymerase comprising the amino acid sequence of SEQ ID NO: 1. In some such embodiments, such a modified TdT polymerase comprises one or more functional differences such as, for example, improved thermostability, or a difference in protease resistance as compared to a wild-type TdT polymerase and/or the TdT polymerase set forth in SEQ ID NO: 1.

In some embodiments, a modified TdT polymerase is or comprises a TdT variant. In some embodiments, a modified TdT polymerase is or comprises a chimeric TdT polymerase. In some embodiments, a chimeric TdT polymerase is a variant TdT polymerase, a chimeric variant polymerase, but a variant TdT polymerase is not necessarily a chimeric TdT polymerase.

In some embodiments, a modified TdT polymerase is a circularly permuted TdT (cpTdT) polymerase. In some embodiments, a cpTdT polymerase is derived from and/or has a sequence that is derived from one or more parental TdT polymerases. In some embodiments, a cpTdT as provided herein is derived from a single parental TdT. In some embodiments, a parental TdT is or comprises a modified TdT polymerase, such as a truncated, variant, and/or chimeric TdT polymerase.

### TdT Variant Polymerases

The present disclosure provides TdT variant polymerases. As used herein, a “TdT variant polymerase” or a “TdT variant” refers to a TdT polymerase comprising at least one amino acid change (e.g., substitution, deletion, modification) relative to that of SEQ ID NO: 1. In some embodiments, a TdT variant comprises a sequence having a certain percentage identity to that of SEQ ID NO: 1 as set forth herein (see also, e.g., Palluk et al. Nature Biotechnology. 36(7):645-650 2018). In some such embodiments, a TdT variant as provided herein comprises an amino acid sequence having at least 60%, at least 65%, at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or more identity to the amino acid sequence set forth in SEQ ID NO: 1. In some embodiments, a TdT variant comprises the amino acid sequence set forth in SEQ ID NO: 1. In some embodiments, a TdT variant comprises one or more amino acid substitutions relative to the TdT polymerase of

SEQ ID NO: 1.
>Terminal deoxynucleotidyl transferase (TdT) (SEQ ID NO: 1)
(SEQ ID NO: 1)
MGGRDIVDGSEFSPSPVPGSQNVPAPAVKKISQYACQRRITLNNYNQLFTDALDIL
AENDELRENEGSALAFMRASSVLKSLPFPITSMKDTEGIPSLGDKVKSIIEGIIEDGESSEA
KAVLNDERYKSFKLFTSVFGVGLKTAEKWFRMGFRTLSKIQSDKSLRFTQMQKAGFLYYEDL
VSCVNRPEAEAVSMLVKEAVVTFLPDALVTMTGGFRRGKMTGHDVDFLITSPEATEDEEQQL
LHKVTDFWKQQGLLLYADILESTFEKFKQPSRKVDALDHFQKCFLILKLDHGRVHSEKSGQQ
EGKGWKAIRVDLVMSPYDRRAFALLGWTGSRQFERDLRRYATHERKMMLDNHALYDRTKRVE
LEAESEEEIFAHLGLDYIEPWERNA

In some embodiments, a modified TdT polymerase is selected from any of those set forth in Table 2 or a variant thereof.

As used herein, the term “sequence identity” has a standard meaning in the art. As is known in the art, a variety of different programs can be used to determine whether a polynucleotide or polypeptide has sequence identity or similarity to a known sequence. Sequence identity or similarity can be determined using standard techniques known in the art, including, but not limited to, the Smith-Waterman Local Sequence Identity Search Algorithm, Adv. Appl. Math. 2: 482 (1981), Needleman-Wunsch homology region alignment algorithm, J. Mol. Biol. 48: 443 (1970), Pearson-Lipman similarity search method, Proc. Natl. Acad. Sci. USA 85: 2444 (1988), using a computerized implementation of these algorithms (GAP, BESTFIT, FASTA, and TFASTA in the Wisconsin Genetics Software Package, Genetics Computer Group, 575 Science Dr., Madison, WI), a best-fit sequence search program, described by Devereux et al., Nucl. Acid Res. 12: 387 (1984), preferably using default parameters, or by visual inspection. Publicly available web-based applications for determining sequence identity are available, (e.g. blast.ncbi.nlm.nih.gov/Blast.cgi). Sequence identity can refer to a percentage of identity over an entire molecule, or a portion thereof. For example, as will be understood by those in the art, a particular polypeptide may have a particular percent of sequence identity to a portion of a molecule, which means that, in some embodiments, in reference to the entire molecule, a sequence identity will be lower relative to percent identity to a portion of the molecule (e.g., sequence identity to an entire vector as compared to a portion of the vector).

In some embodiments, the one or more amino acid substitutions in a modified TdT polymerase as provided herein is at an amino acid position numbered with respect to the amino acid sequence of SEQ ID NO: 1.

In some embodiments, the one or more amino acid substitutions comprises C36V, G108E, A118V, and/or K157E.

In some embodiments, the one or more amino acid substitutions comprises or consists of C36V, G108E, A118V, and K157E.

In some such embodiments, the one or more amino acid substitutions comprises D54, D60, L62, S68, S76, T92, 195, S104, G108, 1110, F148, F166, D206, M211, T235, C36, E64, A118, K157, Q241, C285, E338, 1158, P187, D260, S319, M73, E145, S163, Q168, P187, Q271, C285, G294, M352, L262, R335, V7, L175, N185, M194, K300, R361, E368, E58, C183, M73, R217, K90, K101, K119, R364, D280, D280, D354, D354, H356, L141, D277, A278, R335, R339, R342, A69, T134, E61, S32P, M194, E368, R339, T346, H347, S163, E66, R361, and/or K141.

In some embodiments, the one or more amino acid substitutions comprises A52C, D54E, D54C, D60A, L62F, L62C, S68R, L70C, S76A, S76C, T92L, I95L, S104R, G108E, I110L, K131C, F148Y, F166L, D206G, M211L, T235G, C36V, E64G, A118V, K157E, Q241R, C285A, E338N, 1158V, P187S, D260N, S319P, M73Q, E145K, S163T, Q168E, P187A, Q271L, C285G, G294Q, M352L, L262A, R335T, V7D, L175K, N185T, M194V, K300E, R361K, E368G, E58C, C183G, M73C, R217C, K90E, K101E, K119M, R364E, D280A, D280N, D354A, D354L, H356V, H356A, L141C, D277C, A278C, R335C, R339C, R342C, A69C, T134C, E61C, S32P, M194T, E368K, R339W, T346N, H347Q, S163S, E66C, R361K, and/or K141C.

In some embodiments, the one or more amino acid substitutions comprises V7D, L62F, T92L, E145K, F148Y, I158V, Q168E, L175K, C183G, N185T, Q271L, G294Q, K300E, S319P, and/or M352L.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, and/or M352L.

In some embodiments, the one or more amino acid substitutions comprises C285G, L262A, R335T, and/or R361K.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, and/or R361K. In some embodiments, the one or more amino acid substitutions comprises Q241R.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, and/or Q241R.

In some embodiments, the one or more amino acid substitutions comprises M194V and/or E368G.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, and/or E368G.

In some embodiments, the one or more amino acid substitutions comprises S32P and/or M194T.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S32P and/or M194T.

In some embodiments, the one or more amino acid substitutions comprises S163T.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R and/or S163T.

In some embodiments, the one or more amino acid substitutions comprises E64G.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or E64G.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, and/or E64G.

In some embodiments, the one or more amino acid substitutions comprises C285A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or C285A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, and/or C285A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, and/or C285A.

In some embodiments, the one or more amino acid substitutions comprises E338N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or E338N.

In some embodiments, the one or more amino acid substitutions comprises C36V, N185T, Q271L, G294Q, K300E, S319P, M352L, C285G, L262A, R335T, R361K, Q241R, M194V, E368G, E64G, and/or E338N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, and/or E338N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, and/or E388N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, and/or E338N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, and/or E338N.

In some embodiments, the one or more amino acid substitutions comprises M73C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T and/or M73C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, and/or M73C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, and/or M73C.

In some embodiments, the one or more amino acid substitutions comprises C36V, N185T, Q271L, G294Q, K300E, S319P, M352L, C285G, L262A, R335T, R361K, Q241R S163T, E64G, C285A, and/or M73C.

In some embodiments, the one or more amino acid substitutions comprises D260N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, and/or D260N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, and/or D260N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, and/or D260N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, and/or D260N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, and/or D260N.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, and/or D260N.

In some embodiments, the one or more amino acid substitutions comprises M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or M73Q.

In some embodiments, the one or more amino acid substitutions comprises C36V, N185T, Q271L, G294Q, K300E, S319P, M352L, C285G, L262A, R335T, R361K, Q241R, S163T, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E338N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, D260N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, D260N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, D260N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, D260N, and/or M73Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, D260N, and/or M73Q.

In some embodiments, the one or more amino acid substitutions comprises E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, and/or E58C.

In some embodiments, the one or more amino acid substitutions comprises A69C, T134C, E61C, D277C, A278C, R339C, and/or R342C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, A69C, T134C, E61C, D277C, A278C, R339C, and/or R342C.

In some embodiments, the one or more amino acid substitutions comprises P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, and/or E338N, P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E338N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, D260N, and/or P187S.

In some embodiments, the one or more amino acid substitutions comprises C36V, N185T, Q271L, G294Q, K300E, S319P, M352L, C285G, L262A, R335T, R361K, Q241R, M194V, E368G, E64G, E338N, D260N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, D260N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, D260N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, D260N, and/or P187S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, D260N, and/or P187S.

In some embodiments, the one or more amino acid substitutions comprises P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, and/or P187A.

In some embodiments, the one or more amino acid substitutions comprises C36V, N185T, Q271L, G294Q, K300E, S319P, M352L, C285G, L262A, R335T, R361K, Q241R S163T, E64G, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E338N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, D260N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, D260N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, D260N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, D260N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, D260N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, D260N, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, M73Q, and/or P187A.

In some embodiments, the one or more amino acid substitutions comprises C36V, N185T, Q271L, G294Q, K300E, S319P, M352L, C285G, L262A, R335T, R361K, Q241R, M194V, E368G, E338N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E338N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, D260N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, D260N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, D260N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, D260N, M73Q, and/or P187A.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, D260N, M73Q, and/or P187A.

In some embodiments, the one or more amino acid substitutions comprises D280A, D280N, D354L, or H356V.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, D280A or D280N, D354L, and/or H356V.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, M73C, D280A or D280N, D354L, and/or H356V.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, D280A or D280N, D354L, and/or H356V.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, D280A or D280N, D354L, and/or H356V.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, M73C, D280A or D280N, D354L, and/or H356V.

In some embodiments, the one or more amino acid substitutions comprises R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E338N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, D260N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, D260N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, D260N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, D260N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, D260N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, D260N, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, M73Q, R364E, K90E, K101E, and/or K119M.

In some embodiments, the one or more amino acid substitutions comprises C36V, N185T, Q271L, G294Q, K300E, S319P, M352L, C285G, L262A, R335T, R361K, Q241R, S163T, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E338N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, D260N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, D260N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, D260N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, D260N, M73Q, R364E, K90E, K101E, and/or K119M.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, D260N, M73Q, R364E, K90E, K101E, and/or K119M.

In some embodiments, the one or more amino acid substitutions comprises R339W, T346N, and/or H347Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, R339W, T346N, and/or H347Q.

In some embodiments, the one or more amino acid substitutions comprises C36V, N185T, Q271L, G294Q, K300E, S319P, M352L, C285G, L262A, R335T, R361K, Q241R, S32P, M194T, R339W, T346N, and/or H347Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, R339W, T346N, and/or H347Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, M73C, R339W, T346N, and/or H347Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, M73C, R339W, T346N, and/or H347Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, M73C, R339W, T346N, and/or H347Q.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, M73C, R339W, T346N, and/or H347Q.

In some embodiments, the one or more amino acid substitutions comprises E66C and R361K.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, E66C and/or R361K.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S32P, M194T, E66C, and/or R361K.

In some embodiments, the one or more amino acid substitutions comprises R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E338N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, and/or R217C.

In some embodiments, the one or more amino acid substitutions comprises C36V, N185T, Q271L, G294Q, K300E, S319P, M352L, C285G, L262A, R335T, R361K, Q241R S163T, E64G, C285A, E338N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, D260N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, D260N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, D260N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, D260N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, D260N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, D260N, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E338N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E338N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, M73Q, and/or R217C.

In some embodiments, the one or more amino acid substitutions comprises C36V, N185T, Q271L, G294Q, K300E, S319P, M352L, C285G, L262A, R335T, R361K, Q241R, M194V, E368G, E338N, D260N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, E338N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, E338N, D260N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, E388N, D260N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, E338N, D260N, M73Q, and/or R217C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, E338N, D260N, M73Q, and/or R217C. In some embodiments, the one or more amino acid substitutions comprises L141C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or L141C.

In some embodiments, the one or more amino acid substitutions comprises R335C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or R335C.

In some embodiments, the one or more amino acid substitutions comprises E368K.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, and/or E368G.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, C285A, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, M194V, E368G, E64G, C285A, and/or E58C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R S163T, E64G, C285A, and/or E58C.

In some embodiments, the one or more amino acid substitutions comprises S163S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, and/or S163S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S32P, M194T, and/or S163S.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, A69C, T134C, E61C, D277C, A278C, R339C, R342C, and/or S163S.

In some embodiments, the one or more amino acid substitutions comprises K141C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, and/or K141C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S32P, M194T and/or K141C.

In some embodiments, the one or more amino acid substitutions comprises C36V, G108E, A118V, K157E, V7D, L62F, T92L, E145K, F148Y, I158V, Q168E, L175K, C183G, N185T, Q271L, G294Q, K300E, S319P, M352L, C285G, L262A, R335T, R361K, Q241R, S163T, and/or K141C.

In some embodiments, the one or more amino acid substitutions comprises V7D, C36V, D54C, L62F, T92L, G108E, A118V, E145K, F148Y, K157E, 1158V, Q168E, L175K, C183G, N185T, M194V, Q241R, L262A, Q271L, C285G, G294Q, K300E, S319P, R335T, M352L, R361K, and/or E368G.

In some embodiments, the one or more amino acid substitutions comprises V7D, C36V, L62C, T92L, G108E, A118V, E145K, F148Y, K157E, I158V, Q168E, L175K, C183G, N185T, M194V, Q241R, L262A, Q271L, C285G, G294Q, K300E, S319P, R335T, M352L, R361K, and/or E368G.

In some embodiments, the one or more amino acid substitutions comprises V7D, C36V, L62F, L70C, T92L, G108E, A118V, E145K, F148Y, K157E, I158V, Q168E, L175K, C183G, N185T, M194V, Q241R, L262A, Q271L, C285G, G294Q, K300E, S319P, R335T, M352L, R361K, and/or E368G.

In some embodiments, the one or more amino acid substitutions comprises V7D, C36V, L62F, S76C, T92L, G108E, A118V, E145K, F148Y, K157E, 1158V, Q168E, L175K, C183G, N185T, M194V, Q241R, L262A, Q271L, C285G, G294Q, K300E, S319P, R335T, M352L, R361K, and/or E368G.

In some embodiments, the one or more amino acid substitutions comprises V7D, C36V, L62F, T92L, G108E, A118V, E145K, F148Y, K131C, K157E, I158V, Q168E, L175K, C183G, N185T, M194V, Q241R, L262A, Q271L, C285G, G294Q, K300E, S319P, R335T, M352L, R361K, E368G.

In some embodiments, the one or more amino acid substitutions comprises V7D, C36V, A52C, L62F, T92L, G108E, A118V, E145K, F148Y, K157E, 1158V, Q168E, L175K, C183G, N185T, M194V, Q241R, L262A, Q271L, C285G, G294Q, K300E, S319P, R335T, M352L, R361K, E368G.

In some embodiments, the one or more amino acid substitutions comprises a set of substitutions selected from Table 2. It is understood that the amino acid substitutions set forth in Table 2 are with respect to amino acid positions of SEQ ID NO: 1.

TABLE 2
Exemplary Modified TdT Polymerases

| Set | Set of Substitutions |
|---|---|
| 1 | C36V, G108E, A118V, and K157E |
| 2 | D54E, D60A, L62F, S68R, S76A, T92L, I95L, S104R, G108E, I110L, |
| | F148Y, F166L, D206G, M211L, T235G |
| 3 | C36V, E64G, S68R, G108E, A118V, K157E, Q241R, C285A, E338N |
| 4 | C36V, E64G, S68R, G108E, A118V, K157E, Q241R, C285A |
| 5 | C36V, S68R, G108E, A118V, K157E, Q241R, C285A, E338N |
| 6 | C36V, E64G, S68R, G108E, A118V, K157E, Q241R |
| 7 | C36V, E64G, S68R, G108E, A118V, K157E, I158V, P187S, Q241R, D260N, |
| | C285A, S319P, E338N |
| 8 | C36V, L62F, E64G, M73Q, T92L, G108E, A118V, E145K, F148Y, K157E, |
| | I158V, S163T, Q168E, P187A, Q241R, D260N, Q271L, C285G, G294Q, |
| | S319P, E338N, M352L |
| 9 | C36V, S68R, G108E, A118V, K157E, Q241R, L262A, C285A, R335T |
| 10 | V7D, C36V, L62F, M73Q, T92L, G108E, A118V, E145K, F148Y, K157E, |
| | I158V, Q168E, L175K, N185T, M194V, Q241R, Q271L, C285G, G294Q, |
| | K300E, S319P, M352L, R361K, E368G |
| 11 | C36V, E58C, E64G, S68R, G108E, A118V, K157E, C183G, Q241R, C285A, |
| | R335T |
| 12 | C36V, E58C, S68R, G108E, A118V, K157E, C183G, Q241R, L262A, C285A, |
| | R335T |
| 13 | V7D, C36V, L62F, M73C, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 14 | C36V, S68R, M73C, G108E, A118V, K157E, C183G, Q241R, L262A, C285A, |
| | R335T |
| 15 | V7D, C36V, L62F, E64G, M73Q, T92L, G108E, A118V, E145K, F148Y, |
| | K157E, I158V, Q168E, L175K, C183G, N185T, M194V, R217C, Q241R, |
| | D260N, Q271L, C285G, G294Q, K300E, S319P, E338N, M352L, R361K, |
| | E368G |
| 16 | V7D, C36V, L62F, E64G, M73Q, K90E, T92L, K101E, G108E, A118V, |
| | K119M, E145K, F148Y, K157E, I158V, Q168E, L175K, N185T, M194V, |
| | Q241R, D260N, Q271L, C285G, G294Q, K300E, S319P, E338N, M352L, |
| | R361K, R364E, E368G |
| 17 | V7D, C36V, L62F, M73C, T92L, G108E, A118V, E145K, F148Y, K157E, |
| | I158V, Q168E, L175K, C183G, N185T, M194V, Q241R, L262A, Q271L, |
| | D280A, C285G, G294Q, K300E, S319P, R335T, M352L, R361K, E368G |
| 18 | V7D, C36V, L62F, M73C, T92L, G108E, A118V, E145K, F148Y, K157E, |
| | I158V, Q168E, L175K, C183G, N185T, M194V, Q241R, L262A, Q271L, |
| | D280N, C285G, G294Q, K300E, S319P, R335T, M352L, R361K, E368G |
| 19 | V7D, C36V, L62F, M73C, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, D354A, R361K, E368G |
| 20 | V7D, C36V, L62F, M73C, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, D354L, R361K, E368G |
| 21 | V7D, C36V, L62F, M73C, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, H356V, R361K, E368G |
| 22 | V7D, C36V, E61C, L62F, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 23 | V7D, C36V, E61C, L62F, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 24 | V7D, C36V, L62F, A69C, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 25 | V7D, C36V, L62F, T92L, G108E, A118V, T134C, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 26 | V7D, C36V, L62F, T92L, G108E, A118V, L141C, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 27 | V7D, C36V, L62F, T92L, G108E, A118V, E145K, F148Y, K157E, I158V, |
| | Q168E, L175K, C183G, N185T, M194V, Q241R, L262A, Q271L, D277C, |
| | G294Q, K300E, S319P, R335T, M352L, R361K, E368G |
| 28 | V7D, C36V, L62F, T92L, G108E, A118V, E145K, F148Y, K157E, I158V, |
| | Q168E, L175K, C183G, N185T, M194V, Q241R, L262A, Q271L, A278C, |
| | G294Q, K300E, S319P, R335T, M352L, R361K, E368G |
| 29 | V7D, C36V, L62F, T92L, G108E, A118V, E145K, F148Y, K157E, I158V, |
| | Q168E, L175K, C183G, N185T, M194V, Q241R, L262A, Q271L, C285G, |
| | G294Q, K300E, S319P, R335C, M352L, R361K, E368G |
| 30 | V7D, C36V, L62F, T92L, G108E, A118V, E145K, F148Y, K157E, I158V, |
| | Q168E, L175K, C183G, N185T, M194V, Q241R, L262A, Q271L, C285G, |
| | G294Q, K300E, S319P, R335T, R339C, M352L, R361K, E368G |
| 31 | V7D, C36V, L62F, T92L, G108E, A118V, E145K, F148Y, K157E, I158V, |
| | Q168E, L175K, C183G, N185T, M194V, Q241R, L262A, Q271L, C285G, |
| | G294Q, K300E, S319P, R335T, R342C, M352L, R361K, E368G |
| 32 | V7D, S32P, C36V, L62F, M73C, T92L, G108E, A118V, E145K, F148Y, |
| | K157E, I158V, S163T, Q168E, L175K, C183G, N185T, M194T, L262A, |
| | Q271L, C285G, G294Q, K300E, S319P, R335T, M352L, R361K |
| 33 | V7D, S32P, C36V, L62F, M73C, T92L, G108E, A118V, E145K, F148Y, |
| | K157E, I158V, S163T, Q168E, L175K, C183G, N185T, M194T, L262A, |
| | Q271L, C285G, G294Q, K300E, S319P, R335T, R339W, T346N, H347Q, |
| | M352L, R361K |
| 34 | V7D, S32P, C36V, E61C, L62F, T92L, G108E, A118V, E145K, F148Y, |
| | K157E, I158V, S163S, Q168E, L175K, C183G, N185T, M194T, L262A, |
| | Q271L, C285G, G294Q, K300E, S319P, R335T, M352L, R361K |
| 35 | V7D, S32P, C36V, L62F, E66C, T92L, G108E, A118V, E145K, F148Y, |
| | K157E, I158V, S163T, Q168E, L175K, C183G, N185T, M194T, L262A, |
| | Q271L, C285G, G294Q, K300E, S319P, R335T, M352L, R361K |
| 36 | V7D, S32P, C36V, L62F, A69C, T92L, G108E, A118V, E145K, F148Y, |
| | K157E, I158V, S163T, Q168E, L175K, C183G, N185T, M194T, L262A, |
| | Q271L, C285G, G294Q, K300E, S319P, R335T, M352L, R361K |
| 37 | V7D, S32P, C36V, L62F, T92L, G108E, A118V, T134C, E145K, F148Y, |
| | K157E, I158V, S163T, Q168E, L175K, C183G, N185T, M194T, L262A, |
| | Q271L, C285G, G294Q, K300E, S319P, R335T, M352L, R361K |
| 38 | V7D, S32P, C36V, L62F, T92L, G108E, A118V, K141C, E145K, F148Y, |
| | K157E, I158V, S163T, Q168E, L175K, C183G, N185T, M194T, L262A, |
| | Q271L, C285G, G294Q, K300E, S319P, R335T, M352L, R361K |
| 39 | V7D, S32P, C36V, L62F, T92L, G108E, A118V, E145K, F148Y, K157E, |
| | I158V, S163T, Q168E, L175K, C183G, N185T, M194T, L262A, Q271L, |
| | D277C, C285G, G294Q, K300E, S319P, R335T, M352L, R361K |
| 40 | V7D, S32P, C36V, L62F, T92L, G108E, A118V, E145K, F148Y, K157E, |
| | I158V, S163T, Q168E, L175K, C183G, N185T, M194T, L262A, Q271L, |
| | A278C, C285G, G294Q, K300E, S319P, R335T, M352L, R361K |
| 41 | V7D, S32P, C36V, L62F, T92L, G108E, A118V, E145K, F148Y, K157E, |
| | I158V, S163T, Q168E, L175K, C183G, N185T, M194T, L262A, Q271L, |
| | C285G, G294Q, K300E, S319P, R335T, M352L, R361K |
| 42 | V7D, S32P, C36V, L62F, T92L, G108E, A118V, E145K, F148Y, K157E, |
| | I158V, S163T, Q168E, L175K, C183G, N185T, M194T, L262A, Q271L, |
| | C285G, G294Q, K300E, S319P, R335T, R339C, M352L, R361K |
| 43 | V7D, S32P, C36V, L62F, T92L, G108E, A118V, E145K, F148Y, K157E, |
| | I158V, S163T, Q168E, L175K, C183G, N185T, M194T, L262A, Q271L, |
| | C285G, G294Q, K300E, S319P, R335T, R342C, M352L, R361K |
| 44 | V7D, C36V, E58C, L62F, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368K |
| 45 | V7D, C36V, D54C, L62F, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 46 | V7D, C36V, L62C, T92L, G108E, A118V, E145K, F148Y, K157E, I158V, |
| | Q168E, L175K, C183G, N185T, M194V, Q241R, L262A, Q271L, C285G, |
| | G294Q, K300E, S319P, R335T, M352L, R361K, E368G |
| 47 | V7D, C36V, L62F, L70C, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 48 | V7D, C36V, L62F, S76C, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |
| 49 | V7D, C36V, L62F, T92L, G108E, A118V, E145K, F148Y, K131C, K157E, |
| | I158V, Q168E, L175K, C183G, N185T, M194V, Q241R, L262A, Q271L, |
| | C285G, G294Q, K300E, S319P, R335T, M352L, R361K, E368G |
| 50 | V7D, C36V, A52C, L62F, T92L, G108E, A118V, E145K, F148Y, K157E, |
| | I158V, Q168E, L175K, C183G, N185T, M194V, Q241R, L262A, Q271L, |
| | C285G, G294Q, K300E, S319P, R335T, M352L, R361K, E368G |

### Chimeric TdT Variant Polymerases

In some embodiments, a modified TdT polymerase of the present disclosure is a chimeric TdT variant polymerase or chimeric variant polymerase. As used herein a “chimeric TdT” or a “chimeric TdT variant” refers to a modified TdT variant polymerase that comprises a homologous nucleotidyl transferase active site motif that differs by at least one amino acid from that of the amino acids corresponding to positions 332-335 of SEQ ID NO: 1. That is, as will be appreciated in accordance with the present disclosure, a chimeric TdT does not comprise a sequence consisting of TGSR (SEQ ID NO: 125) at amino acid positions corresponding to positions 332-335 of SEQ ID NO: 1. As used herein, “active site motif” refers to a region in a sequence of an enzyme (e.g., TdT polymerase) where substrate molecules are capable of binding and which facilitates a chemical reaction (e.g. nucleotide extension reaction). In some such embodiments, an active site motif is highly conserved and/or structurally superimposable but not necessarily identical among different organisms. For example, amino acid positions 332-335 of the polymerase of SEQ ID NO: 1 represent an active site motif. That is, in some embodiments, a polymerase comprising of having the sequence as set forth in SEQ ID NO: 1 also comprise an active site motif at amino acid positions corresponding to positions 332-335 of SEQ ID NO: 1.

In some embodiments, the chimeric TdT variant comprises a homologous nucleotidyl transferase active site motif derived from a species other than Mus musculus. In some embodiments, the TdT variant provided herein comprises a homologous nucleotidyl transferase active site motif from a non-bovine species. In some embodiments, the TdT variant provided herein comprises a homologous nucleotidyl transferase active site motif from a non-human species.

In some embodiments, the chimeric TdT variant comprises a homologous nucleotidyl transferase active site motif from a species selected from the group comprising or consisting of Tinamus guttatus (GenBank: KGL73349.1; SEQ ID NO: 5), Eudromia elegans (GenBank: NXA34558.1; SEQ ID NO: 6), Nothoprocta perdicaria (NCBI Reference Sequence: XP_025906782.1; SEQ ID NO: 7), Nothoprocta ornate (GenBank: NWY00334.1; SEQ ID NO: 8), Nothoprocta pentlandii (GenBank: NWX83107.1; SEQ ID NO: 9), Nothocercus Julius (GenBank: NXA48721.1; SEQ ID NO: 10), Nothocercus nigrocapillus (GenBank: NXD14109.1; SEQ ID NO: 11), Crypturellus undulatus (GenBank: NWJ05551.1; SEQ ID NO: 12), Crypturellus soui (GenBank: NWI08830.1; SEQ ID NO: 13), Serilophus lunatus (GenBank: NXM66262.1; SEQ ID NO: 14), Gouania willdenow (NCBI Reference Sequence: XP_028324800.1; SEQ ID NO: 15), Silurus meridionalis (GenBank: KAF7701041.1; SEQ ID NO: 16), Coregonus clupeaformis (NCBI Reference Sequence: XP_041735872.1; SEQ ID NO: 17), Oncorhynchus nerka (NCBI Reference Number: XP_029496749.1; SEQ ID NO: 18), Vicugna pacos (XP_006210640.1; SEQ ID NO: 19), Elephantulus edwardii (NCBI Reference Sequence: XP_006880141.1; SEQ ID NO: 20), Galeopterus variegatus (NCBI Reference Sequence: XP_008573590.1; SEQ ID NO: 21), Engystomops pustulosus (GenBank: KAG8551716.1; SEQ ID NO: 22), Bufo bufo (NCBI Reference Sequence: XP_040294203.1; SEQ ID NO: 23), Bufo gargarizans (NCBI Reference Sequence: XP_044155657.1; SEQ ID NO: 24), Sciurus carolinensis (GenBank: MBZ3884855.1; SEQ ID NO: 25), Nanorana perkeri (NCBI Reference Sequence: XP_018414400.1; SEQ ID NO: 26), Carassius auratus (NCBI Reference Sequence: XP_026079168.1; SEQ ID NO: 27), Pimephales promelas (NCBI Reference Sequence: XP_039504943.1; SEQ ID NO: 28), Chanos chanos (NCBI Reference Sequence: XP_030628137.1; SEQ ID NO: 29), Python bivittatus (NCBI Reference Sequence: XP_007424457.1; SEQ ID NO: 30), and Danio rerio (GenBank: AAS89780.1; SEQ ID NO: 31).

In some embodiments, the homologous nucleotidyl transferase active site motif corresponds to amino acid positions 332-335 of SEQ ID NO: 1. In some embodiments, the homologous nucleotidyl transferase active site motif is selected from the group consisting of TGSK (SEQ ID NO: 121), TGSP (SEQ ID NO: 122), TGSQ (SEQ ID NO: 123), and TGST (SEQ ID NO: 124). In some embodiments, the homologous nucleotidyl transferase active site motif comprises TGSK (SEQ ID NO: 121). In some embodiments, the homologous nucleotidyl transferase active site motif comprises TGSP (SEQ ID NO: 122). In some embodiments, the homologous nucleotidyl transferase active site motif comprises TGSQ (SEQ ID NO: 123). In some embodiments, the homologous nucleotidyl transferase active site motif comprises TGST (SEQ ID NO: 124).

In some embodiments, a chimeric TdT is a variant TdT, but a variant TdT is not necessarily a chimeric TdT if there is not at least one amino acid difference in amino acids corresponding to positions 332-335 of SEQ ID NO: 1.

In some embodiments, the present disclosure provides chimeric TdT variants comprising homologous TdT sequences derived from two or more organisms. In some embodiments, the chimeric TdT variants comprise a first sequence component and a second sequence component. In some embodiments, the first sequence component is from a first organism. In some embodiments, the second sequence is from a second organism. In some embodiments, the first organism is from a different species as the second organism. In some embodiments, the first organism is from a different genus as the second organism (and, thus, a different species). The first and second sequences can be composed in various arrangements. For example, first and second sequences can be arranged in a contiguous fashion with the N-terminal of the first sequence at the N-terminal side of the TdT chimeric molecule, and the N-terminal of the second sequence arranged following the C-terminal of the first sequence. In some embodiments, the first sequence can be arranged N-terminal to the second sequence, or vice versa. The first sequence can be arranged with its C-terminal to the second sequence, or vice versa. The first sequence may be embedded within the second sequence, or vice versa. The first sequence may replace a homologous sequence in the second sequence (e.g. an active site motif), or vice versa.

In some embodiments, the chimeric TdT variant comprises a TdT sequence (e.g., amino acid sequence, nucleotide sequences) identical to a TdT sequence from Mus musculus or having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, a TdT sequence from Mus musculus comprises an N-terminal truncation. In some embodiments, a TdT sequence from Mus musculus comprises one or more amino acid substitutions relative to that of SEQ ID NO: 1.

In some embodiments, the chimeric TdT variant sequence derived from Mus musculus is or comprises SEQ ID NO: 1. In some embodiments, the one or more amino acid substitutions are selected from the group comprising A52C, D54E, D54C, D60A, L62F, L62C, S68R, L70C, S76A, S76C, T92L, I95L, S104R, G108E, I110L, K141C, F148Y, F166L, D206G, M211L, T235G, C36V, E64G, A118V, K157E, Q241R, C285A, E338N, I158V, P187S, D260N, S319P, M73Q, E145K, S163T, Q168E, P187A, Q271L, C285G, G294Q, M352L, L262A, R335T, V7D, L175K, N185T, M194V, K300E, R361K, E368G, E58C, C183G, M73C, R217C, K90E, K101E, K119M, R364E, D280A, D280N, D354A, D354L, H356V, H356A, L141C, D277C, A278C, R335C, R339C, R342C, A69C, T134C, E61C, S32P, M194T, E368K, R339W, T346N, H347Q, S163S, E66C, R361K, K141C, and combinations thereof. In some embodiments, the one or more amino acid substitutions are selected from a set of substitutions set forth in Table 2.

In some embodiments, the first sequence is selected from a non-Mus musculus organism. In some embodiments, the second sequence is selected from a non-Mus musculus organism. In some embodiments, the chimeric TdT variant comprises a sequence from a non-Mus musculus organism selected from the group comprising or consisting of Tinamus guttatus (GenBank: KGL73349.1; SEQ ID NO: 5), Eudromia elegans (GenBank: NXA34558.1; SEQ ID NO: 6), Nothoprocta perdicaria (NCBI Reference Sequence: XP_025906782.1; SEQ ID NO: 7), Nothoprocta ornate (GenBank: NWY00334.1; SEQ ID NO: 8), Nothoprocta pentlandii (GenBank: NWX83107.1; SEQ ID NO: 9), Nothocercus Julius (GenBank: NXA48721.1; SEQ ID NO: 10), Nothocercus nigrocapillus (GenBank: NXD14109.1; SEQ ID NO: 11), Crypturellus undulatus (GenBank: NWJ05551.1; SEQ ID NO: 12), Crypturellus soui (GenBank: NWI08830.1; SEQ ID NO: 13), Serilophus lunatus (GenBank: NXM66262.1; SEQ ID NO: 14), Gouania willdenow (NCBI Reference Sequence: XP_028324800.1; SEQ ID NO: 15), Silurus meridionalis (GenBank: KAF7701041.1; SEQ ID NO: 16), Coregonus clupeaformis (NCBI Reference Sequence: XP_041735872.1; SEQ ID NO: 17), Oncorhynchus nerka (NCBI Reference Number: XP_029496749.1; SEQ ID NO: 18), Vicugna pacos (XP_006210640.1; SEQ ID NO: 19), Elephantulus edwardii (NCBI Reference Sequence: XP_006880141.1; SEQ ID NO: 20), Galeopterus variegatus (NCBI Reference Sequence: XP_008573590.1; SEQ ID NO: 21), Engystomops pustulosus (GenBank: KAG8551716.1; SEQ ID NO: 22), Bufo bufo (NCBI Reference Sequence: XP_040294203.1; SEQ ID NO: 23), Bufo gargarizans (NCBI Reference Sequence: XP_044155657.1; SEQ ID NO: 24), Sciurus carolinensis (GenBank: MBZ3884855.1; SEQ ID NO: 25), Nanorana perkeri (NCBI Reference Sequence: XP_018414400.1; SEQ ID NO: 26), Carassius auratus (NCBI Reference Sequence: XP_026079168.1; SEQ ID NO: 27), Pimephales promelas (NCBI Reference Sequence: XP_039504943.1; SEQ ID NO: 28), Chanos chanos (NCBI Reference Sequence: XP_030628137.1; SEQ ID NO: 29), Python bivittatus (NCBI Reference Sequence: XP_007424457.1; SEQ ID NO: 30), and Danio rerio (GenBank: AAS89780.1; SEQ ID NO: 31).

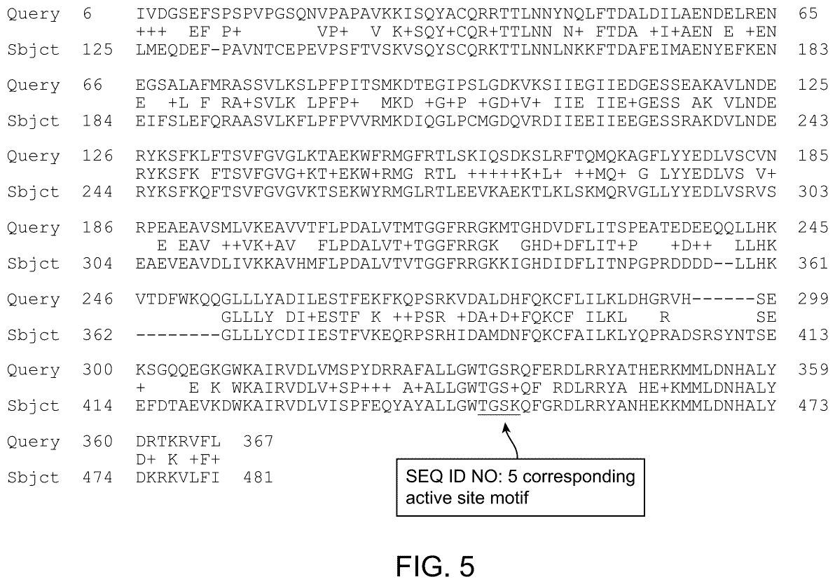

In some embodiments, the sequence from a non-Mus musculus organism comprises an active site motif from a homologous nucleotidyl transferase. In some embodiments, the active site motif of an amino acid sequence of a TdT variant or TdT chimera disclosed herein corresponds to amino acid positions 332-335 of SEQ ID NO: 1. In some embodiments, the homologous nucleotidyl transferase sequence comprises any one of SEQ ID NOs: 5-31. To identify the active site motif corresponding to the homologous nucleotidyl transferase sequence, the active site motif corresponding to positions 332-335 of SEQ ID NO: 1, an alignment can be performed using publicly available computational tools known in the art (e.g. the Basic Local Alignment Search Tool at blast.ncbi.nlm.nih.gov/Blast.cgi). For example, an alignment of SEQ ID NO: 1 and SEQ ID NO: 5 (as shown, in part, in FIG. 5;

FIG. 5 discloses SEQ ID NOS 126 and 127, respectively, in order of appearance)) reveals an active site motif of “TGSK (SEQ ID NO: 121)” at amino acids 446-449, and corresponding to amino acids 332-335 of SEQ ID NO: 1. In some embodiments, the active site motif from a homologous nucleotidyl transferase is selected from the group consisting of TGSK (SEQ ID NO: 121), TGSP (SEQ ID NO: 122), TGSQ (SEQ ID NO: 123), and TGST (SEQ ID NO: 124). In some embodiments, the active site motif from a homologous nucleotidyl transferase comprises TGSK (SEQ ID NO: 121). In some embodiments, the active site motif from a homologous nucleotidyl transferase comprises TGSP (SEQ ID NO: 122). In some embodiments, the active site motif from a homologous nucleotidyl transferase comprises TGSQ (SEQ ID NO: 123). In some embodiments, the active site motif from a homologous nucleotidyl transferase comprises TGST (SEQ ID NO: 124).

### Exemplary Modified TdT Polymerase Sequences

In some embodiments, a modified TdT polymerase of the present disclosure comprises or consists of a sequence selected from any one of SEQ ID NOs: 32-80, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 32, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprise s or consists of the amino acid sequence of SEQ ID NO: 33, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT comprises or consists of the amino acid sequence of SEQ ID NO: 34, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 35, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 36, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 37, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 38, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 39, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 40, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 41, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 42, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 43, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 44, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 45, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 46, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 47, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 48, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 49, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 50, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 51, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 52, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 53, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 54, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the TdT polymerase comprises or consists of the amino acid sequence of SEQ ID NO: 55, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 56, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 57, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 58, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 59, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 60, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 61, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 62, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 63, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 64, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 65, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 66, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 67, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 68, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 69, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 70, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 71, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 72, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 73, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 74, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 75, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 76, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 77, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 78, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 79, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, the modified TdT polymerase comprises or consists of the amino acid sequence of SEQ ID NO: 80, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more, or 100% identity thereto.

In some embodiments, a modified TdT polymerase comprises or consists of a sequence as set forth in any one of SEQ ID NOs 32-80. In some embodiments, a TdT variant comprises or consists of a sequence as set forth in any one of SEQ ID NOs: 32-37, 39, and 44-45. In some embodiments, a chimeric TdT variant comprises or consists of a sequence as set forth in any one of SEQ ID NOs: 38, 40-43, and 46-80.

In some embodiments, a modified TdT polymerase comprising a sequence that consists of TGSR (SEQ ID NO: 125) at positions corresponding to amino acids 332-335 of SEQ ID NO: 1 is considered a TdT variant. In some embodiments, a modified TdT polymerase consisting of TGSX, wherein X is K, P, Q, or T, and wherein X is not R at positions corresponding to amino acids 332-335 of SEQ ID NO: 1 is considered a chimeric TdT variant.

TABLE 3
Exemplary TdT Variant Sequences

| SEQ ID | |
|---|---|
| NO: | Sequence |
| 32 | MGGRDIVDGSEFSPSPVPGSQNVPAPAVKKISQYAVQRRTTLNNYNQLFTDALDILAENDELRGNEGR |
| | ALAFMRASSVLKSLPFPITSMKDTEGIPSLGDKVKSIIEEIIEDGESSEVKAVLNDERYKSFKLFTSV |
| | FGVGLKTAEKWFRMGFRTLSEIQSDKSLRFTQMQKAGFLYYEDLVSCVNRPEAEAVSMLVKEAVVTFL |
| | PDALVTMTGGFRRGKMTGHDVDFLITSPEATEDEEQRLLHKVTDFWKQQGLLLYADILESTFEKFKQP |
| | SRKVDALDHFQKAFLILKLDHGRVHSEKSGQQEGKGWKAIRVDLVMSPYDRRAFALLGWTGSRQFNRD |
| | LRRYATHERKMMLDNHALYDRTKRVFLEAESEEEIFAHLGLDYIEPWERNA |
| 33 | MGGRDIVDGSEFSPSPVPGSQNVPAPAVKKISQYAVQRRTTLNNYNQLFTDALDILAENDELRGNEGR |
| | ALAFMRASSVLKSLPFPITSMKDTEGIPSLGDKVKSIIEEIIEDGESSEVKAVLNDERYKSFKLFTSV |
| | FGVGLKTAEKWFRMGFRTLSEIQSDKSLRFTQMQKAGFLYYEDLVSCVNRPEAEAVSMLVKEAVVTFL |
| | PDALVTMTGGFRRGKMTGHDVDFLITSPEATEDEEQRLLHKVTDFWKQQGLLLYADILESTFEKFKQP |
| | SRKVDALDHFQKAFLILKLDHGRVHSEKSGQQEGKGWKAIRVDLVMSPYDRRAFALLGWTGSRQFERD |
| | LRRYATHERKMMLDNHALYDRTKRVFLEAESEEEIFAHLGLDYIEPWERNA |
| 34 | MGGRDIVDGSEFSPSPVPGSQNVPAPAVKKISQYAVQRRTTLNNYNQLFTDALDILAENDELRENEGR |
| | ALAFMRASSVLKSLPFPITSMKDTEGIPSLGDKVKSIIEEIIEDGESSEVKAVLNDERYKSFKLFTSV |
| | FGVGLKTAEKWFRMGFRTLSEIQSDKSLRFTQMQKAGFLYYEDLVSCVNRPEAEAVSMLVKEAVVTFL |
| | PDALVTMTGGFRRGKMTGHDVDFLITSPEATEDEEQRLLHKVTDFWKQQGLLLYADILESTFEKFKQP |
| | SRKVDALDHFQKAFLILKLDHGRVHSEKSGQQEGKGWKAIRVDLVMSPYDRRAFALLGWTGSRQFNRD |
| | LRRYATHERKMMLDNHALYDRTKRVFLEAESEEEIFAHLGLDYIEPWERNA |
| 35 | MGGRDIVDGSEFSPSPVPGSQNVPAPAVKKISQYAVQRRTTLNNYNQLFTDALDILAENDELRGNEGR |
| | ALAFMRASSVLKSLPFPITSMKDTEGIPSLGDKVKSIIEEIIEDGESSEVKAVLNDERYKSFKLFTSV |
| | FGVGLKTAEKWFRMGFRTLSEIQSDKSLRFTQMQKAGFLYYEDLVSCVNRPEAEAVSMLVKEAVVTEL |
| | PDALVTMTGGFRRGKMTGHDVDFLITSPEATEDEEQRLLHKVTDFWKQQGLLLYADILESTFEKFKQP |
| | SRKVDALDHFQKCFLILKLDHGRVHSEKSGQQEGKGWKAIRVDLVMSPYDRRAFALLGWTGSRQFNRD |
| | LRRYATHERKMMLDNHALYDRTKRVFLEAESEEEIFAHLGLDYIEPWERNA |
| 36 | MGGRDIVDGSEFSPSPVPGSQNVPAPAVKKISQYAVQRRTTLNNYNQLFTDALDILAENDELRGNEGR |
| | ALAFMRASSVLKSLPFPITSMKDTEGIPSLGDKVKSIIEEIIEDGESSEVKAVLNDERYKSFKLFTSV |
| | FGVGLKTAEKWFRMGFRTLSEVQSDKSLRFTQMQKAGFLYYEDLVSCVNRSEAEAVSMLVKEAVVTFL |
| | PDALVTMTGGFRRGKMTGHDVDFLITSPEATEDEEQRLLHKVTDFWKQQGLLLYANILESTFEKFKQP |
| | SRKVDALDHFQKAFLILKLDHGRVHSEKSGQQEGKGWKAIRVDLVMPPYDRRAFALLGWTGSRQFNRD |
| | LRRYATHERKMMLDNHALYDRTKRVFLEAESEEEIFAHLGLDYIEPWERNA |
| 37 | MGGRDIVDGSEFSPSPVPGSQNVPAPAVKKISQYAVQRRTTLNNYNQLFTDALDILAENDEFRGNEGS |
| | ALAFQRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFLYYEDLVSCVNRAEAEAVSMLVKEAVVTFL |
| | PDALVTMTGGFRRGKMTGHDVDFLITSPEATEDEEQRLLHKVTDFWKQQGLLLYANILESTFEKFKLP |
| | SRKVDALDHFQKGFLILKLDHQRVHSEKSGQQEGKGWKAIRVDLVMPPYDRRAFALLGWTGSRQFNRD |
| | LRRYATHERKMLLDNHALYDRTKRVFLEAESEEEIFAHLGLDYIEPWERNA |
| 38 | MGGRDIVDGSEFSPSPVPGSQNVPAPAVKKISQYAVQRRTTLNNYNQLFTDALDILAENDELRENEGR |
| | ALAFMRASSVLKSLPFPITSMKDTEGIPSLGDKVKSIIEEIIEDGESSEVKAVLNDERYKSFKLFTSV |
| | FGVGLKTAEKWFRMGFRTLSEIQSDKSLRFTQMQKAGFLYYEDLVSCVNRPEAEAVSMLVKEAVVTFL |
| | PDALVTMTGGFRRGKMTGHDVDFLITSPEATEDEEQRLLHKVTDFWKQQGLLLYADIAESTFEKFKQP |
| | SRKVDALDHFQKAFLILKLDHGRVHSEKSGQQEGKGWKAIRVDLVMSPYDRRAFALLGWTGSTQFERD |
| | LRRYATHERKMMLDNHALYDRTKRVFLEAESEEEIFAHLGLDYIEPWERNA |
| 39 | MGGRDIDDGSEFSPSPVPGSQNVPAPAVKKISQYAVQRRTTLNNYNQLFTDALDILAENDEFRENEGS |
| | ALAFQRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSCVTRPEAEAVSVLVKEAVVTFL |
| | PDALVTMTGGFRRGKMTGHDVDFLITSPEATEDEEQRLLHKVTDFWKQQGLLLYADILESTFEKFKLP |
| | SRKVDALDHFQKGFLILKLDHQRVHSEESGQQEGKGWKAIRVDLVMPPYDRRAFALLGWTGSRQFERD |
| | LRRYATHERKMLLDNHALYDKTKRVFLGAESEEEIFAHLGLDYIEPWERNA |
| 40 | MGGRDIVDGSEFSPSPVPGSQNVPAPAVKKISQYAVQRRTTLNNYNQLFTDALDILACNDELRGNEGR |
| | ALAFMRASSVLKSLPFPITSMKDTEGIPSLGDKVKSIIEEIIEDGESSEVKAVLNDERYKSFKLFTSV |
| | FGVGLKTAEKWFRMGFRTLSEIQSDKSLRFTQMQKAGFLYYEDLVSGVNRPEAEAVSMLVKEAVVTFL |
| | PDALVTMTGGFRRGKMTGHDVDFLITSPEATEDEEQRLLHKVTDFWKQQGLLLYADILESTFEKFKQP |
| | SRKVDALDHFQKAFLILKLDHGRVHSEKSGQQEGKGWKAIRVDLVMSPYDRRAFALLGWTGSTQFERD |
| | LRRYATHERKMMLDNHALYDRTKRVFLEAESEEEIFAHLGLDYIEPWERNA |
| 41 | MGGRDIVDGSEFSPSPVPGSQNVPAPAVKKISQYAVQRRTTLNNYNQLFTDALDILACNDELRENEGR |
| | ALAFMRASSVLKSLPFPITSMKDTEGIPSLGDKVKSIIEEIIEDGESSEVKAVLNDERYKSFKLFTSV |
| | FGVGLKTAEKWFRMGFRTLSEIQSDKSLRFTQMQKAGFLYYEDLVSGVNRPEAEAVSMLVKEAVVTFL |
| | PDALVTMTGGFRRGKMTGHDVDFLITSPEATEDEEQRLLHKVTDFWKQQGLLLYADIAESTFEKFKQP |
| | SRKVDALDHFQKAFLILKLDHGRVHSEKSGQQEGKGWKAIRVDLVMSPYDRRAFALLGWTGSTQFERD |
| | LRRYATHERKMMLDNHALYDRTKRVFLEAESEEEIFAHLGLDYIEPWERNA |
| 42 | MGGRDIDDGSEFSPSPVPGSQNVPAPAVKKISQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 43 | MGGRDIVDGSEFSPSPVPGSQNVPAPAVKKISQYAVQRRTTLNNYNQLFTDALDILAENDELRENEGR |
| | ALAFCRASSVLKSLPFPITSMKDTEGIPSLGDKVKSIIEEIIEDGESSEVKAVLNDERYKSFKLFTSV |
| | FGVGLKTAEKWFRMGFRTLSEIQSDKSLRFTQMQKAGFLYYEDLVSGVNRPEAEAVSMLVKEAVVTFL |
| | PDALVTMTGGFRRGKMTGHDVDFLITSPEATEDEEQRLLHKVTDFWKQQGLLLYADIAESTFEKFKQP |
| | SRKVDALDHFQKAFLILKLDHGRVHSEKSGQQEGKGWKAIRVDLVMSPYDRRAFALLGWTGSTQFERD |
| | LRRYATHERKMMLDNHALYDRTKRVFLEAESEEEIFAHLGLDYIEPWERNA |
| 44 | MGGRDIDDGSEFSPSPVPGSQNVPAPAVKKISQYAVQRRTTLNNYNQLFTDALDILAENDEFRGNEGS |
| | ALAFQRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRCGKMTGHDVDFLITSPEATEDEEQRLLHKVTDFWKQQGLLLYANILESTFEKFKLP |
| | SRKVDALDHFQKGFLILKLDHQRVHSEESGQQEGKGWKAIRVDLVMPPYDRRAFALLGWTGSRQFNRD |
| | LRRYATHERKMLLDNHALYDKTKRVFLGAESEEEIFAHLGLDYIEPWERNA |
| 45 | MGGRDIDDGSEFSPSPVPGSQNVPAPAVKKISQYAVQRRTTLNNYNQLFTDALDILAENDEFRGNEGS |
| | ALAFQRASSVLKSLPFPITSMEDLEGIPSLGDEVKSIIEEIIEDGESSEVMAVLNDERYKSFKLFTSV |
| | FGVGLKTAKKWYRMGFRTLSEVQSDKSLRFTEMQKAGFKYYEDLVSCVTRPEAEAVSVLVKEAVVTFL |
| | PDALVTMTGGFRRGKMTGHDVDFLITSPEATEDEEQRLLHKVTDFWKQQGLLLYANILESTFEKFKLP |
| | SRKVDALDHFQKGFLILKLDHQRVHSEESGQQEGKGWKAIRVDLVMPPYDRRAFALLGWTGSRQFNRD |
| | LRRYATHERKMLLDNHALYDKTKEVFLGAESEEEIFAHLGLDYIEPWERNA |
| 46 | MGGRDIDDGSEFSPSPVPGSQNVPAPAVKKISQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALAHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 47 | MGGRDIDDGSEFSPSPVPGSQNVPAPAVKKISQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALNHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 48 | MGGRDIDDGSEFSPSPVPGSQNVPAPAVKKISQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLANHALYDKTKRVFLGAESEEEIFAHLGLDYIEPWERNA |
| 49 | MGGRDIDDGSEFSPSPVPGSQNVPAPAVKKISQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLLNHALYDKTKRVFLGAESEEEIFAHLGLDYIEPWERNA |
| 50 | MGGRDIDDGSEFSPSPVPGSQNVPAPAVKKISQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNAALYDKTKRVFLGAESEEEIFAHLGLDYIEPWERNA |
| 51 | MGGRDIDDGSEFSPSPVPGSQNVPAPAVKKISQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNVALYDKTKRVFLGAESEEEIFAHLGLDYIEPWERNA |
| 52 | MGGRDIDDGSEFSPSPVPGSQNVPAPAVKKISQYAVQRRTTLNNYNQLFTDALDILAENDCFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 53 | MGGRDIDDGSEFSPSPVPGSQNVPAPAVKKISQYAVQRRTTLNNYNQLFTDALDILAENDEFRENCGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 54 | MGGRDIDDGSEFSPSPVPGSQNVPAPAVKKISQYAVQRRTTLNNYNQLFTDALDILAENDEFRENEGS |
| | CLAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 55 | MGGRDIDDGSEFSPSPVPGSQNVPAPAVKKISQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFCSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 56 | MGGRDIDDGSEFSPSPVPGSQNVPAPAVKKISQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGCKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 57 | MGGRDIDDGSEFSPSPVPGSQNVPAPAVKKISQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVCALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 58 | MGGRDIDDGSEFSPSPVPGSQNVPAPAVKKISQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDCLDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 59 | MGGRDIDDGSEFSPSPVPGSQNVPAPAVKKISQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSCQFERD |
| | LRRYATHERKMLLDNHALYDKTKRVFLGAESEEEIFAHLGLDYIEPWERNA |
| 60 | MGGRDIDDGSEFSPSPVPGSQNVPAPAVKKISQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFECD |
| | LRRYATHERKMLLDNHALYDKTKRVFLGAESEEEIFAHLGLDYIEPWERNA |
| 61 | MGGRDIDDGSEFSPSPVPGSQNVPAPAVKKISQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LCRYATHERKMLLDNHALYDKTKRVFLGAESEEEIFAHLGLDYIEPWERNA |
| 62 | MGGRDIDDGSEFSPSPVPGSQNVPAPAVKKIPQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLEAESEEEIFAHLGLDYIEPWERNA |
| 63 | MGGRDIDDGSEFSPSPVPGSQNVPAPAVKKIPQYAVQRRTTLNNYNQLFTDALDILAENDEFRENEGS |
| | ALAFCRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFEWD |
| | LRRYANQERKMLLDNHALYDKTKRVFLEAESEEEIFAHLGLDYIEPWERNA |
| 64 | MGGRDIDDGSEFSPSPVPGSQNVPAPAVKKIPQYAVQRRTTLNNYNQLFTDALDILAENDCFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLEAESEEEIFAHLGLDYIEPWERNA |
| 65 | MGGRDIDDGSEFSPSPVPGSQNVPAPAVKKIPQYAVQRRTTLNNYNQLFTDALDILAENDEFRENCGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLEAESEEEIFAHLGLDYIEPWERNA |
| 66 | MGGRDIDDGSEFSPSPVPGSQNVPAPAVKKIPQYAVQRRTTLNNYNQLFTDALDILAENDEFRENEGS |
| | CLAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLEAESEEEIFAHLGLDYIEPWERNA |
| 67 | MGGRDIDDGSEFSPSPVPGSQNVPAPAVKKIPQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFCSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLEAESEEEIFAHLGLDYIEPWERNA |
| 68 | MGGRDIDDGSEFSPSPVPGSQNVPAPAVKKIPQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGCKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLEAESEEEIFAHLGLDYIEPWERNA |
| 69 | MGGRDIDDGSEFSPSPVPGSQNVPAPAVKKIPQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVCALDHFQKGFLILKLDHQRVHSEESGQQEGKGWKAIRVDLVMPPYDRRAFALLGWTGSTQFERD |
| | LRRYATHERKMLLDNHALYDKTKRVFLEAESEEEIFAHLGLDYIEPWERNA |
| 70 | MGGRDIDDGSEFSPSPVPGSQNVPAPAVKKIPQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDCLDHFQKGFLILKLDHQRVHSEESGQQEGKGWKAIRVDLVMPPYDRRAFALLGWTGSTQFERD |
| | LRRYATHERKMLLDNHALYDKTKRVFLEAESEEEIFAHLGLDYIEPWERNA |
| 71 | MGGRDIDDGSEFSPSPVPGSQNVPAPAVKKIPQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSCQFERD |
| | LRRYATHERKMLLDNHALYDKTKRVFLEAESEEEIFAHLGLDYIEPWERNA |
| 72 | MGGRDIDDGSEFSPSPVPGSQNVPAPAVKKIPQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFECD |
| | LRRYATHERKMLLDNHALYDKTKRVFLEAESEEEIFAHLGLDYIEPWERNA |
| 73 | MGGRDIDDGSEFSPSPVPGSQNVPAPAVKKIPQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKTLRFTEMQKAGFKYYEDLVSGVTRPEAEAVSTLVKEAVVTFL |
| | PDALVTMTGGFRRGKMTGHDVDFLITSPEATEDEEQQLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LCRYATHERKMLLDNHALYDKTKRVFLEAESEEEIFAHLGLDYIEPWERNA |
| 74 | MGGRDIDDGSEFSPSPVPGSQNVPAPAVKKISQYAVQRRTTLNNYNQLFTDALDILACNDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 75 | MGGRDIDDGSEFSPSPVPGSQNVPAPAVKKISQYAVQRRTTLNNYNQLFTDALCILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 76 | MGGRDIDDGSEFSPSPVPGSQNVPAPAVKKISQYAVQRRTTLNNYNQLFTDALDILAENDECRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 77 | MGGRDIDDGSEFSPSPVPGSQNVPAPAVKKISQYAVQRRTTLNNYNQLFTDALDILAENDEFRENEGS |
| | ACAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 78 | MGGRDIDDGSEFSPSPVPGSQNVPAPAVKKISQYAVQRRTTLNNYNQLFTDALDILAENDEFRENEGS |
| | ALAFMRACSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 79 | MGGRDIDDGSEFSPSPVPGSQNVPAPAVKKISQYAVQRRTTLNNYNQLFTDALDILAENDEFRENEGS |
| | ALAFMRASSVLKSLPFPITSMKDLEGIPSLGDKVKSIIEEIIEDGESSEVKAVLNDERYKSFCLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |
| 80 | MGGRDIDDGSEFSPSPVPGSQNVPAPAVKKISQYAVQRRTTLNNYNQLFTDCLDILAENDEFRENEGS |
| | ALAFMRASSVLKSLPFPITSMKDLEGIPSLGDKVKSIIEEIIEDGESSEVKAVLNDERYKSFKLFTSV |
| | FGVGLKTAKKWYRMGFRTLSEVQSDKSLRFTEMQKAGFKYYEDLVSGVTRPEAEAVSVLVKEAVVTFL |
| | PDALVTMTGGFRRGKMTGHDVDFLITSPEATEDEEQRLLHKVTDFWKQQGLLLYADIAESTFEKFKLP |
| | SRKVDALDHFQKGFLILKLDHQRVHSEESGQQEGKGWKAIRVDLVMPPYDRRAFALLGWTGSTQFERD |
| | LRRYATHERKMLLDNHALYDKTKRVFLGAESEEEIFAHLGLDYIEPWERNA |

In some embodiments, the modified TdT polymerase comprises an active site motif. In some embodiments, the active site motif has a sequence comprising the amino acids corresponding to positions 332-335 of SEQ ID NO: 1. In some embodiments, the active site motif is a homologous nucleotidyl transferase active site motif. In some embodiments, the active site motif is optionally substituted. For example, in some embodiments, the active site motif comprises or consists of an amino acid sequence that varies by one or more amino acids relative to the amino acids corresponding to those at positions 332-335 of SEQ ID NO: 1. In some such embodiments, a modified TdT polymerase comprising an active site motif that differs by one or more amino acids from those amino acids corresponding to positions 332-335 of SEQ ID NO: 1 is a chimeric TdT variant polymerase.

In some embodiments, the modified TdT polymerase comprises a tag for visualization, detection, functional characterization, enrichment, isolation, cell and/or tissue targeting, and/or cell and/or tissue permeability.

In some embodiments, the modified TdT polymerase is a fusion protein. For example, in some embodiments, a modified TdT polymerase can be a fusion protein, that is, for example, fused to a fluorescent marker, a histidine-tag (His-tag), a maltose binding protein (MBP), or an albumin. In some such embodiments, the present disclosure contemplates that such fusions may allow for or facilitate features and/or properties such as improved purification and isolation, expression in a host cell, improved solubility, and/or one or more other beneficial properties (see, e.g. Mishra, Curr Protein Pept Sci. 2020; 21(8):821-830.; and Waugh, Postepy Biochem. 2016; 62(3):377-382).

In some embodiments, the modified TdT polymerase is fused to a maltose binding protein (MBP).

Exemplary Maltose Binding Protein
(SEQ ID NO: 81)
MKIEEGKLVIWINGDKGYNGLAEVGKKFEKDTGIKVTVEHPDKLEEKFPQVAATGDG
PDIIFWAHDRFGGYAQSGLLAEITPDKAFQDKLYPFTWDAVRYNGKLIAYPIAVEALSLIYN
KDLLPNPPKTWEEIPALDKELKAKGKSALMENLQEPYFTWPLIAADGGYAFKYENGKYDIKD
VGVDNAGAKAGLIFLVDLIKNKHMNADTDYSIAEAAFNKGETAMTINGPWAWSNIDTSKVNY
GVTVLPTFKGQPSKPFVGVLSAGINAASPNKELAKEFLENYLLTDEGLEAVNKDKPLGAVAL
KSYEEELVKDPRIAATMENAQKGEIMPNIPQMSAFWYAVRTAVINAASGRQTVDEALKDAQT

In some embodiments, a fusion protein of the present disclosure comprises a linker region. In some embodiments, when expressed as a fusion protein, a modified TdT polymerase in accordance with the present disclosure may comprise a linker region joining the modified TdT polymerase sequence and the sequence to which it is fused, such as provided in an exemplary linker region set forth in SEQ ID NO: 82. A linker can be any suitable amino acid sequence (see, e.g. Chen et al. Adv Drug Deliv Rev. 2013 Oct. 15; 65(10): 1357-1369.,).

In some embodiments, the linker region comprises a cleavage site. In some embodiments, the cleavage site is a proteolytic cleavage site.

Exemplary linker region
(SEQ ID NO: 82)
NSSSNNNNNNNNNNLGIEGRISHMS

In some embodiments, the modified TdT polymerase comprises a His-tag, such as that exemplified by the His-tag of SEQ ID NO: 83.

Exemplary His-tag
(SEQ ID NO: 83)
MGHHHHHHHHHHSSGHIDDDDKHM

### Circularly Permuted Polypeptides

Circularly permuted proteins were first discovered in nature in 1979 by Bruce Cunningham and colleagues. (Cunningham B A, et al. Proc Natl Acad Sci 76: 3218-3222, 1979. A review of circularly permuted variants of proteins and their history is provided in Bliven et al. (PloS Comput Biol. 8(3), 2012).

As used herein, the terms “circularly permuted,” “circular permutation,” and variations thereof refer to an engineered or modified polynucleotide or polypeptide (i.e., a molecule having a linear primary structure), wherein the molecule comprises two terminal components (e.g., a 5′ end and a 3′ end, e.g. an N-terminus and a C-terminus, etc.) that are or have been joined together, either directly or indirectly such as, e.g., via a linker, to produce a substantially circular/circularized molecule, which, after initial circularization, is then opened at another location on the circularized molecule, which location is distinct from the location where two termini of the linear molecule were initially joined. The resulting molecule is a new substantially linear molecule with a second set of two termini that differ from the first set of two termini in the first joining (i.e., in the “original” or “native” or “parent” molecule, e.g., a parent TdT). As provided herein circular permutation retains at least a portion of amino acid or nucleic acid sequence identity with that of the parent molecule, but in a rearranged order (i.e., sequence) relative to the parent, generating new termini (relative to those of the starting molecule).

In accordance with the present disclosure, in some embodiments, circular permutation includes any process that results in a circularly permuted protein. Circular permutation and processes to generate circularly permuted molecules are generally known to those of skill in the art.

In some embodiments, design of a sequence (e.g., a nucleic acid sequence, e.g., an amino acid sequence) for a circularly permuted DNA, RNA, or protein comprises joining terminal ends of a “parent” sequence. As used herein, the term “parent” refers to a “starting” sequence or molecule (e.g., polynucleotide or polypeptide) that, relative to the cpTdT has, at some point, been circularly permuted. In some embodiments, a circularly permuted molecule is produced from a parent or polynucleotide parent by joining the natural (i.e., as found in nature) N- and C-termini of a given parent polypeptide (or the 5′ and 3′ termini for a polynucleotide parent). In some embodiments, a parent polypeptide is truncated at the N- and/or C-termini (or the parent polynucleotides is truncated at the 5′ and 3′ termini) prior to being joined to produce a circularly permuted molecule. In some embodiments, a portion of a parent polypeptide is truncated at the N- and/or C-termini (or the parent polynucleotides is truncated at the 5′ and 3′ termini) prior to being joined to produce a circularly permutated molecule. In some embodiments, an assessment of the protein structure of the molecule is performed to determine effects (e.g., advantages) of truncating the termini of the protein prior to joining the termini. A non-limiting example of an assessment includes determining a terminal end to be non-essential to the functional domains to be preserved in a circular permutation molecule prior to truncating the termini of a parent and joining the termini.

In some embodiments, a linker is used to join the termini. Publicly available tools for designing linkers that join the N- and C-termini in designing circular permutations are generally known to those of skill in the art (see, for example, Chen et al. BMC Bioinformatics. October 12; 22(Suppl 10). 2021).

In some embodiments, once the initial terminal end joining has occurred, the circularized parent molecule may then be opened at any place in its structure. For example, the circularized parent molecule can be opened at a position in its sequence between known tertiary structures or domains of the protein, such as unstructured loop regions. Alternatively, a circularized molecule can be opened at a site within known secondary, tertiary structures, or domains (e.g., of a polypeptide, e.g., of a polynucleotide). In some embodiments, the circularized parent molecule is opened at any position within a sequence to generate new N- and C-termini in a protein's polypeptide sequence.

In some embodiments, circularly permuted molecules are designed in silico and/or synthesized de novo and do not necessarily require production from a parental molecule. That is, in some embodiments, a circularly permuted molecule (e.g., a cpTdT) is synthesized de novo as a linear molecule and is not subjected to a specific circularization and opening step.

Circularly Permuted TdT (cpTdT) Polymerases

In some embodiments, a circularly permuted polypeptide is a modified TdT polymerase that is a circularly permuted TdT polymerase. In some such embodiments, a cpTdT has one or more parental TdT polymerases. In some embodiments, one or more regions of a parent TdT comprise a suitable opening point for generating circularly permutated variants (i.e., cpTdTs). For instance, by way of non-limiting example, in some embodiments, a suitable opening point is at an amino acid position separating secondary structures. In some embodiments, an opening point is not at or does not comprise a conserved region or a functional domain of a parent molecule (e.g., a parent TdT).

By way of non-limiting example, in some embodiments, a region (i.e. amino acid positions) corresponding to an opening point comprises a sequence having at least 60%, at least 70%, at least 80%, at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to amino acid positions 38-46, amino acid positions 63-65, amino acid positions 81-88, amino acid positions 93-100, amino acid positions 113-115, amino acid positions 135-140, amino acid positions 150-154, amino acid positions 161-167, amino acid positions 176-177, amino acid positions 182-185, amino acid positions 204-207, amino acid positions 217-219, amino acid positions 231-235, amino acid positions 254-255, amino acid positions 263-267, amino acid positions 271-274, amino acid positions 278-281, amino acid positions 296-305, amino acid positions 319-320, amino acid positions 333-334, amino acid positions 354-357, amino acid positions 368-371, and amino acid positions 380-385 relative to the amino acid sequence of the TdT polymerase of SEQ ID NO: 1.

In some embodiments, one or more regions of a TdT structure (i.e., in a parent TdT) that serve as suitable opening points in generating circular permutation variants (e.g., cpTdTs) are at amino acid positions separating functional domains of TdT. Certain functional domains of TdT are generally known to those of skill in the art (see, e.g. Loc'h and Delarue. Curr Opin Struct Biol. 2018 December; 53:22-31). In some embodiments, a functional domain comprises a highly conserved region of a wild type TdT polymerase. Non-limiting examples of functional domains of TdT include: BRCA1 carboxy-terminal (BRCT) domain, SD1, SD2, HhH motif, 5′ phosphate binding site, and rNTP steric gate. In some embodiments, a region comprising an opening point (e.g., an amino acid position) corresponds to a sequence having at least 60%, at least 70%, at least 80%, at least 90%, at least 95%, at least 97%, at least 98%, at least 99% identity, at least 99.1%, at least 99.2%, at least 99.3%, at least 99.4%, at least 99.5%, at least 99.6%, at least 99.7%, at least 99.8%, at least 99.9% or 100% identity to amino acid positions 1-45, amino acid positions 112-131, amino acid positions 181-185, or amino acid positions 257-325 relative to the amino acid sequence of the TdT polymerase of SEQ ID NO: 1.

In some embodiments, one or more regions of a parental TdT that are suitable opening points for generating circular permutation variants (e.g., cpTdTs) are at amino acid positions between both a secondary structure and a functional domains of wild type TdTs. In some embodiments, a region (i.e. amino acid positions) corresponding to an opening point comprises a sequence having at least 60%, at least 70%, at least 80%, at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to amino acid positions 38-45, amino acid positions 113-115, amino acid positions 182-185, amino acid positions 263-267, amino acid positions 271-274, amino acid positions 278-281, amino acid positions 296-305, and amino acid positions 319-320 relative to the amino acid sequence of the TdT polymerase of SEQ ID NO: 1.

In some embodiments, a region (i.e. amino acid positions) corresponding to opening points as described herein is identified in any wild type TdT or modified TdT polymerase by performing a sequence alignment with SEQ ID NO: 1. In some embodiments, an alignment is analyzed to determine the corresponding amino acid position(s) in a wild type TdT or modified TdT polymerase that align with the region (i.e. amino acid positions) corresponding to opening points of SEQ ID NO: 1 described herein.

In some embodiments, design and characterization of one or more cpTdTs, components thereof, and/or polypeptides thereof or related thereto (e.g., parental TdTs, etc.) include sequence design, preparation of nucleic acids encoding a circular permutated molecule, expression vectors, expression and isolation of a circularly permuted protein, subsequent biophysical and/or functional characterization, and combinations thereof including, but not limited to, as provided herein.

Among other things, the present disclosure provides circularly permuted polypeptides comprising template-independent polymerase activity. In some embodiments, a circularly permuted polypeptide as provided herein comprises a sequence derived from one or more terminal deoxynucleotidyl transferases (TdTs). As used herein, a sequence is “derived” from another sequence if it has certain features of that sequence such as, for example, certain percent identity to that sequence. For example, in some embodiments, a sequence of a cpTdT derived from a TdT (i.e. a parental TdT) is a sequence having at least 90%, at least 95%, at least 97%, at least 98%, at least 99% identity, at least 99.1%, at least 99.2%, at least 99.3%, at least 99.4%, at least 99.5%, at least 99.6%, at least 99.7%, at least 99.8%, at least 99.9% or 100% identity to two or more portions of a sequence of a parental TdT polymerase (e.g., a wild type TdT, e.g., a modified TdT), wherein the sequence order of the portions are rearranged relative to the sequence of the parental TdT polymerase.

In some embodiments, a wild type TdT is any TdT or nucleotidyl transferase sequence that is found in nature (e.g., natively expressed in any living organism). In some embodiments, a modified TdT polymerase is or comprises any such modified TdT polymerase as provided herein or known in the art. For instance, certain exemplary modified TdT polymerases can be found in, for example, WO 2001/064909 A1, U.S. Pat. No. 7,494,797, WO 2018/217689 A1, WO 2018/215803 A1, WO 2020/072715 A1, WO 2020/081985 A1, WO 2020/099451 A1, WO 2020/161480 A1, WO 2020/239737 A1, WO 2021/094251 A1, WO 2021/116270 A1.

In some embodiments, a modified TdT polymerase as provided herein (e.g., a TdT variant, e.g., a chimeric TdT variant) comprises a sequence having at least 90%, at least 95%, at least 96%, at least 97%, at least 98%, or at least 99%, at least 99.1%, at least 99.2%, at least 99.3%, at least 99.4%, at least 99.5%, at least 99.6%, at least 99.7%, at least 99.8%, at least 99.9% identity to SEQ ID NO: 1. In some embodiments, a modified TdT polymerase comprises or consists of a sequence having at least 60%, at least 65%, at least 70%, at least 75%, at least 80%, at least 85%, at least 90%, at least 95%, at least 96%, at least 97%, at least 98%, or at least 99% identity to SEQ ID NO: 1.

In some embodiments, a parental TdT comprises SEQ ID NO: 1. In some embodiments, a parental TdT is or comprises a modified TdT polymerase. In some embodiments, a modified TdT polymerase comprises one or more amino acid substitutions relative to a reference TdT (e.g., a modified TdT, e.g., a TdT of SEQ ID NO: 1, e.g., a wild type TdT, etc.). In some embodiments, a modified TdT polymerase comprises or consists of a sequence having at least 90%, at least 95%, at least 96%, at least 97%, at least 98%, at least 99% identity, at least 99.1%, at least 99.2%, at least 99.3%, at least 99.4%, at least 99.5%, at least 99.6%, at least 99.7%, at least 99.8%, at least 99.9% or 100% identity to an amino acid sequence selected from the group comprising SEQ ID NOs: 32-74.

In some embodiments, cpTdTs as provided herein comprises an N- and/or C-terminal truncation relative to a parental TdT. In some such embodiments, a cpTdT comprises an N-terminal truncation. In some embodiments, the N-terminal truncation comprises removal or absence of about 1 to about 170 N-terminal amino acids relative to the sequence of the parental TdT. In some embodiments, an N-terminal truncation comprises removal of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, or about 170 N-terminal amino acids of a wild type TdT polymerase or a modified TdT polymerase. In some embodiments, an N-terminal truncation comprises removal of about 160 amino acids of a wild type TdT polymerase. In some embodiments, an N-terminal truncation comprises removal of about 20 amino acids from a modified TdT polymerase.

In some embodiments, a truncation includes removal or absence of a particular defined sequence motif that is present in a parental TdT such as a wild type TdT or a modified TdT as provided herein. For example, in some embodiments, an N-terminal truncation comprises removal or absence of a region comprising an N-terminal amino acid through a defined sequence motif of a wild type TdT or a modified TdT polymerase, wherein the sequence motif is YX1CQRX2TX3 (SEQ ID NO: 84), where X1 is A or S, X2 is K or R, and X3 is P or T. For example, the wild type TdT sequence for Homo sapiens is set forth in SEQ ID NO: 2 and the YX1CQRX2TX3 (SEQ ID NO: 84) sequence motif is at amino acid positions 153-160. In some embodiments, an N-terminal truncation of SEQ ID NO: 2, comprising or consisting of removal or absence of a region having an amino acid sequence comprising an N-terminal amino acid through the sequence motif YX1CQRX2TX3 (SEQ ID NO: 84), will not include amino acids 1-160 (numbered relative to SEQ ID NO: 2), resulting in SEQ ID NO: 85.

(SEQ ID NO: 85)
LNNCNQIFTDAFDILAENCEFRENEDSCVTFMRAASVLKSLPFTIISMKDTEGIPCLGSK
VKGIIEEIIEDGESSEVKAVLNDERYQSFKLFTSVFGVGLKTSEKWFRMGFRTLSKVRSDKSLKF
TRMQKAGFLYYEDLVSCVTRAEAEAVSVLVKEAVWAFLPDAFVTMTGGFRRGKKMGHDVDFLITS
PGSTEDEEQLLQKVMNLWEKKGLLLYYDLVESTFEKLRLPSRKVDALDHFQKCFLIFKLPRORVD
SDQSSWQEGKTWKAIRVDLVLCPYERRAFALLGWTGSRQFERDLRRYATHERKMILDNHALYDKT
KRIFLKAESEEEIFAHLGLDYIEPWERNA.

In some embodiments, an N-terminal truncation comprises or consists of removal or absence of amino acids MGGRDIDDGSEFSPSPVPGSQNVPAPAVKKISQYAVQRRTT (SEQ ID NO: 86), or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, the terminal ends of a parent TdT polymerase as disclosed herein are joined by a linker. In some embodiments, a linker comprises a gly-leu (“GL”) linkage. In some embodiments, a linker comprises a YPRAGP (SEQ ID NO: 87) linkage.

In some embodiments, a circularly permuted polypeptide comprises or consists of the amino acid sequence set forth in SEQ ID NO: 88, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

(SEQ ID NO: 88)
MEQQEGKGWKAIRVDLVMPPYDRRAFALLGWTGSRQFNRDLRRYATHERKMLLDNHALYD
RTKEVFLEAESEEEIFAHLGLDYIEPWERNAGLLNNYNQLFTDALDILAENDEFRGNEGSALAFQ
RASSVLKSLPFPITSMEDLEGIPSLGDEVKSIIEEIIEDGESSEVMAVLNDERYKSFKLFTSVFG
VGLKTAKKWYRMGFRTLSEVQSDKTLRFTEMQKAGFLYYEDLVSCVNRAEAEAVSMLVKEAVVTF
LPDALVTMTGGFRRGKMTGHDVDFLITSPEATEDEEQRLLHKVTDFWKQQGLLLYANILESTFEK
FKLPSRKVDGLDHFQKGFLILKLDHGRVHSEES.

In some embodiments, the circularly permuted polypeptide comprises or consists of the amino acid sequence as set forth in SEQ ID NO: 89, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

(SEQ ID NO: 89)
SLGDKVKSIIEEIIEDGESSEVKAVLNDERYKSFKLFTSVFGVGLKTAKKWYRMGFRTLS
EVQSDKSLRFTEMQKAGFKYYEDLVSGVTRPEAEAVSVLVKEAVVTFLPDALVTMTGGFRRGKMT
GHDVDFLITSPEATEDEEQRLLHKVTDFWKQQGLLLYADIAESTFEKFKLPSRKVDALDHFQKGF
LILKLDHQRVHSEESGQQEGKGWKAIRVDLVMPPYDRRAFALLGWTGSTQFERDLRRYATHERKM
LLDNHALYDKTKRVFLGAESEEEIFAHLGLDYIEPWERNAYPRAGPLNNYNQLFTDALDILAEND
EFRENEGSALAFCRASSVLKSLPFPITSMKDLEGIP.

In some embodiments, a circularly permuted polypeptide comprises or consists of the amino acid sequence set forth in SEQ ID NO: 90, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

(SEQ ID NO: 90)
SLGDKVKSIIEEIIEDGESSEVKAVLNDERYKSFKLFTSVFGVGLKTAKKWYRMGERTLS
EVQSDKSLRFTEMQKAGFKYYEDLVSGVTRPEAEAVSVLVKEAVVTFLPDALVTMTGGERRGKMT
GHDVDFLITSPEATEDEEQRLLHKVTDFWKQQGLLLYADIAESTFEKFKLPSRKVDALDHFQKGF
LILKLDHQRVHSEESGQQEGKGWKAIRVDLVMPPYDRRAFALLGWTGSTQFERDLRRYATHERKM
LLDNHALYDKTKRVFLGAESEEEIFAHLGLDYIEPWERNAGLLNNYNQLFTDALDILAENDEFRE
NEGSALAFCRASSVLKSLPFPITSMKDLEGIP.

In some embodiments a circularly permuted polypeptide comprises or consists of an amino acid sequence having at least 90%, at least 95%, at least 96%, at least 97%, at least 98%, at least 99% identity, at least 99.1%, at least 99.2%, at least 99.3%, at least 99.4%, at least 99.5%, at least 99.6%, at least 99.7%, at least 99.8%, or at least 99.9% or more or 100% identity to a sequence selected from Table 4.

In some embodiments, a circularly permuted polypeptide comprises or consists of the amino acid sequence set forth in SEQ ID NO: 91, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 92, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 93, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 94, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 95, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 96, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 97, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 98, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 99, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 100, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 101, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 102, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 103, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 104, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 105, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 106, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 107, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 108, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 109, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 110, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 111, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 112, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 113, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 114, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 115, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 116, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 117, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 118, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 118, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a circularly permuted polypeptide comprises or consists of the amino acid sequence set forth in SEQ ID NO: 120, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

Certain exemplary circularly permutated polypeptides (e.g. cpTdTs) and amino acid sequences are provided in Table 4. As will be appreciated, given context, the bracketed [YPRAGP (SEQ ID NO: 87)/GL] in sequences provided herein represent linker alternatives. In some embodiments, a circularly permuted polypeptide of Table 4 comprises a linker having the amino acid sequence YPRAGP (SEQ ID NO: 87). In some embodiments, a circularly permuted polypeptide of Table 4 comprises a gly-leu (“GL”) linkage.

TABLE 4
Exemplary Circularly Permuted Polypeptides

| SEQ ID | | Species |
|---|---|---|
| NO: | Sequence | Derived From |
| 91 | CLGSKVKGIIEEIIEDGESSEVKAVLNDERYQSFKLFTSVFGVGLKTSEKW | Homo sapiens |
| | FRMGFRTLSKVRSDKSLKFTRMQKAGFLYYEDLVSCVTRAEAEAVSVLVKE | |
| | AVWAFLPDAFVTMTGGFRRGKKMGHDVDFLITSPGSTEDEEQLLQKVMNLW | |
| | EKKGLLLYYDLVESTFEKLRLPSRKVDALDHFQKCFLIFKLPRQRVDSDQS | |
| | SWQEGKTWKAIRVDLVLCPYERRAFALLGWTGSRQFERDLRRYATHERKMI | |
| | LDNHALYDKTKRIFLKAESEEEIFAHLGLDYIEPWERNA[YPRAGP/GL]L | |
| | NNCNQIFTDAFDILAENCEFRENEDSCVTFMRAASVLKSLPFTIISMKDTE | |
| | GIP | |
| 92 | CLGDKVKSIIEGIIEDGESSEAKAVLNDERYKSFKLFTSVFGVGLKTAEKW | Mus musculus |
| | FRMGFRTLSKIQSDKSLRFTQMQKAGFLYYEDLVSCVNRPEAEAVSMLVKE | |
| | AVVTFLPDALVTMTGGFRRGKMTGHDVDFLITSPEATEDEEQQLLHKVTDF | |
| | WKQQGLLLYCDILESTFEKFKQPSRKVDALDHFQKCFLILKLDHGRVHSEK | |
| | SGQQEGKGWKAIRVDLVMCPYDRRAFALLGWTGSRQFERDLRRYATHERKM | |
| | MLDNHALYDRTKGKTVTISPLDGKVSKLQKALRVFLEAESEEEIFAHLGLD | |
| | YIEPWERNA[YPRAGP/GL]LNNYNQLFTDALDILAENDELRENEGSCLAF | |
| | MRASSVLKSLPFPITSMKDTEGIP | |
| 93 | CLGDKVKCIIEEIIEDGESSEVKAVLNDERYQSFKLFTSVFGVGLKTSEKW | Bos taurus |
| | FRMGFRSLSKIMSDKTLKFTKMQKAGFLYYEDLVSCVTRAEAEAVGVLVKE | |
| | AVWAFLPDAFVTMTGGFRRGKKIGHDVDFLITSPGSAEDEEQLLPKVINLW | |
| | EKKGLLLYYDLVESTFEKFKLPSRQVDTLDHFQKCFLILKLHHQRVDSSKS | |
| | NQQEGKTWKAIRVDLVMCPYENRAFALLGWTGSRQFERDIRRYATHERKMM | |
| | LDNHALYDKTKRVFLKAESEEEIFAHLGLDYIEPWERNA[YPRAGP/GL]L | |
| | NNYNHIFTDAFEILAENSEFKENEVSYVTFMRAASVLKSLPFTIISMKDTE | |
| | GIP | |
| 94 | CMGDQVRDIIEEIIEEGESSRAKDVLNDERYKSFKQFTSVFGVGVKTSEKW | Tinamus |
| | YRMGLRTLEEVKAEKTLKLSKMQRVGLLYYEDLVSRVSEAEVEAVDLIVKK | guttatus |
| | AVHMFLPDALVTVTGGFRRGKKIGHDIDFLITNPGPRDDDDLLHKGLLLYC | |
| | DIIESTFVKEQRPSRHIDAMDNFQKCFAILKLYQPRADSRSYNTSEEFDTA | |
| | EVKDWKAIRVDLVISPFEQYAYALLGWTGSKQFGRDLRRYANHEKKMMLDN | |
| | HALYDKRKVLFI[YPRAGP/GL]LNNLNKKFTDAFEIMAENYEFKENEIFS | |
| | LEFQRAASVLKFLPFPVVRMKDIQGLP | |
| 95 | CMGDQVRDIIEEIIEEGESSRAKDVLNDERYKSFKQFTSVFGVGVKTSEKW | Eduromia |
| | YRMGLRTLEEVKAEKTLKLSKMQRVGLLYYEDLVTCVSEAEANAVGLIVKK | elegans |
| | AVHTFLPDALVTLTGGFRRGKKIGHDIDFLITNPGPREDDDLLHKGLLLYC | |
| | DIIESTFVKEQRPSRNVDAMDNFQKCFAILKLHQPPADNRSHSTSEEFDMA | |
| | AVRDWKAIRVDLVISPFEQYAYALLGWTGSKQFGRDLRRYASHEKKMMLDN | |
| | HALYDKRKRIFLKAGSEEEIFAHLGLDYVEPWERNA[YPRAGP/GL]LNNF | |
| | NKKFTDAFEIMAENYEFKENEIFSLEFLRAASVLKCLPFPVVRMRDVQGLP | |
| 96 | CMGDQVRDIIEEIIEEGESSRVKDVLNDERYKSFKQFTSVFGVGVKTSEKW | Nothoprocta |
| | YRMGLRTLEEVTAEKTLKLSKMQKVGLLYYEDLVSCVSEAEADAVGLVVKK | perdicaria |
| | AVHTFLPDALVTLTGGFRRGKKIGHDIDFLITNPGPREDDDLLHKVVDLWK | |
| | KQGLLLYCDIIESTFVKEQRPSRNIDAMDNFQKCFAILKLYQPRVDSESQS | |
| | TSEEFDMAEVKDWKAIRVDLVISPFEQYAYALLGWTGSKQFGRDLRRYANH | |
| | EKKMMLDNHALYDRRKRIFLKAGSEEEIFAHLGLDYVEPWERNA[YPRAGP | |
| | /GL]LNNFNKKFTDAFEIMAENYEFKENEIFSLEFLRAASVLKFLPFPVVR | |
| | MKDIQGLP | |
| 97 | CMGDQVRDIIEEIIEEGESSRAKDVLNDDRYKSFKQFTSVFGVGVKTSEKW | Nothoprocta |
| | YRMGLRTLEEVKAEKTLKLSKMQKVGLLYYEDLVSCVSEAEADAVGLVVKK | ornate |
| | AVHTFLPDALVTLIGGERRGKKIGHDIDFLITNPGPREDDDLLHKGLLLYC | |
| | DIIESTFVKEQRPSRNIDAMDNFQKCFAILKLYQPRVDSKSQSTSEELDMA | |
| | EVKDWKAIRVDLVISPFEQYAYALLGWTGSKQFGRDLRRYASHEKKMMLDN | |
| | HALYDRRKRIFLKAGSEEEIFAHLGLDYVEPWERNA[YPRAGP/GL]LNNE | |
| | NKKFTDAFEIMAENYEFKENEIFSLEFLRAASVMKFLPFPVVRMKDIQGLP | |
| 98 | CMGDQVRDIIEEIIEEGESSRAKDVLNDDRYKSFKQFTSVFGVGVKTSEKW | Nothoprocta |
| | YRMGLRTLEEVKAEKTLKLSKMQKVGLLYYEDLVSCVSEAEADAVGLVVKK | pentlandii |
| | AVHTFLPDALVTLTGGFRRGKKIGHDIDFLITNPGPREDDDLLHKGLLLYC | |
| | DIIESTFVKEQRPSRNIDAMDNFQKCFAILKLYQPRVDSKSQSTSEELDMA | |
| | EVKDWKAIRVDLVISPFEQYAYALLGWTGSKQFGRDLRRYASHEKMMMLDN | |
| | HALYDRRKRIFLKAGSEEEIFAHLGLDYVEPWERNA[YPRAGP/GL]INNL | |
| | NKKFTDAFEIMAENYEFKENEIFSLEFLRAASVMKFLPFPVVRMKDIQGLP | |
| 99 | CMGDQVRDIIEEIIEEGESSRAKDVLNDERYKSFKQFTSVFGVGVKTSEKW | Nothocercus |
| | YRMGLRTLEEVKAEKTLKLSKMQRVGLLYYEDLVSCVSEAEVDAVGLIVKK | julius |
| | TVHMFLPDALVTVTGGFRRGKKIGHDIDFLITNPGPREDDDLLHKGLLLYC | |
| | DIIESTFVKEQRPSRNIDAMDNFQKCFAILKLYQPQVDNRSHNTSEEFNTA | |
| | EVKGWKAIRVDLVISPFEQYAYALLGWTGSKQFGRDLRRYANHEKKMMLDN | |
| | HALYDRRKRIFLKAGSEEEIFAHLGLDYLEPWERNA[YPRAGP/GL]LNNF | |
| | NKKFTDAFEIMAENYEFKENEIFSLEFQRAASVLKFLPFPVVRMKDIQGLP | |
| 100 | CMGDHVRDIIEEIIEEGESSRAKDVLNDERYKSFKQFTSVFGVGVKTSEKW | Nothocercus |
| | YRMGLRTLEEVKAEKTLKLSKMQRVGLLYYEDLVSCVSEAEVDAVGLIVKK | nigrocapillus |
| | TVHMFLPDALVTVTGGFRRGKKIGHDIDFLITNPGPREDDDLLHKGLLLYC | |
| | DIIESTFVKEQRPSRNIDAMDNFQKCFAILKLYQPRVDNRSHNTSEEFKAA | |
| | EVKDWKAIRVDLVISPFEQYPYALLGWTGSKQFGRDLRRYANHEKKMMLDN | |
| | HALYDRRKRIFLKAGSEEEIFAHLGLDYLEPWERNA[YPRAGP/GL]LSNF | |
| | NKKFTDAFEIMAENYEFKENEVESLEFQRAASVLKSLSFPVVRMKDIQGLP | |
| 101 | CMGDQVKDIIEEIIEEGESSRTKDVLNDERYKSFKQFTSVFGVGVKTSEKW | Crypturellus |
| | YRMGLRTLEEVKAEKTLKLSKMQRVGLLYYEDLVSCVSEAEVDAVGLIVKK | undulatus |
| | AVHTFLPDALVTVTGGFRRGKKIGHDIDFLITNPGPRDDDDLLHKGLLLYC | |
| | DIIESTFVKEQQPSRNIDAMDNFQKCFAILKLYQPQVDNRSYNTSEECDTA | |
| | EVKDWKAIRVDLVISPFEQYAYALLGWTGSKQFGRDLRRYAHHEKKMMLDN | |
| | HALYDRRKRIFLKAGSEEEIFAHLGLDYVEPWERNA[YPRAGP/GL]LNNM | |
| | NKKFTDAFEIMAENYEFKENEILSLEFQRAASVLKFLPFPVVRMKDIQGLP | |
| 102 | CMGDQVRHIIEEIIEEGESSRAKDVLNDERYKSFKQFTSVFGVGVKTSEKW | Crypturellus |
| | YRMGLRTLEEVKAEKTLKLSKMQRVGLLYYEDLVSCVSEAEVDAVGLIVKK | soui |
| | AVHTFLPDALVTVTGGFRRGKKIGHDIDFLITNPGPREDDDLLHKGLLLYC | |
| | DIIESTFVKEQQPSRNIDAMDNFQKCFAILKLYQPQVDNRSYNASEECDTA | |
| | EVKDWKAIRVDLVISPFEQYAYALLGWTGSKQFGRDLRRYARHEKKMMLDN | |
| | HALYDSRRKIFLKAGSEEEIFAHLGLDYVEPWERNA[YPRAGP/GL]INNL | |
| | NKKFTDAFEIMAENYEFKENEILSLEFQRAASVLKFLPFPVLRMKDIQGLP | |
| 103 | CIGDQVRDIIEEIIEEGESSRVKEVLNDERYKAFKQFTSIFGVGVKTSEKW | Serilophus |
| | YRMGLRTVEELKADKTLKLSKMQKAGFLYYEDLVSCVSKAEADAVTLIVKN | lunatus |
| | TVCTFLPDALVTITGGFRRGKKMGHDIDFLITNPGPREDDELLHKGLLLYC | |
| | DIIESTFVKEQLPSRKVDAMDHFQKCFTILKLYQPRVDNSSYDMSKKLDMA | |
| | EVKDWKAIRVDLVITPFEQYAYALLGWTGSKQFGRDLRRYATHEKKMILDN | |
| | HALYDRKKRIFLKAGSEEEIFAHLGLDYVEPWERNA[YPRAGP/GL]LNNY | |
| | NKKFTDAFEIMAENFEFKENEIFSLEFLRAASLLKSLPFPVTRMKDIQGLP | |
| 104 | FLGKHSIAVIEEILQCGFSMEVQKILSNERFKTLKLFMSVFGVGLKTAEKW | Gouania |
| | YRKGLLSFSDILAAPGIHLNRMQQNGFLHYGDISRAVSKAEAQALGNIIDE | willdenowi |
| | TVHAITSDALLTLTGGFRRGKDFGHDVDFIVTTPEMGKEEHLLTAVIESLK | |
| | HQGLILYCDYRASTFDMSRLPNHKFDAMDHFAKCFLILRLDGSRVEGGLCG | |
| | AEGDGRGWRAVRVDLVSPPMKQYAFTLLGWTGSKQFERDLRRFAQKERHML | |
| | LDNHALYDKTKRELLAATTEQDIFAHLGLQYIEPCQRNA[YPRAGP/GL]T | |
| | ANSNKIFTDAFEVLAESYEFNEMEGQSLAFRRAASVLRSLPSKIQSLRDAE | |
| | SLP | |
| 105 | LKHHNSFLTEALEILAENAEFSENEGRSVAFRRSASVLKALPHAVRTIDEL | Silurus |
| | KDLPCLGEHSLSVIKEILEDGTSREVESTRQSEQFQAMKALTGVFGVGVKT | meridionalis |
| | ADRWVREGLRSPLDLVDTGQKLNHAQQAGVQYYKDLNTPVTRKEAMVISVI | |
| | VEKAVKALLPEAIITLTGGFRRGKEVGHDVDFLITHPEEGAEEKLMPKIVN | |
| | WLEDQGLLLYQKTTRNSYLESKDGPARPPSNMDRFERCFSIFKLEQSKIDS | |
| | NAHAEASSWRAIRVDLVVSPYSQFAFATLGWTGSKLFERELRRWAGQEKGM | |
| | SLSSHALYDSKKKCYLQASSEEEIFTHLGLEYIPPSERNA[YPRAGP/GL] | |
| | LKHHNSFLTEALEILAENAEFSENEGRSVAFRRSASVLKALPHAVRTIDEL | |
| | KDLP | |
| 106 | WLGEHTKAVMEEILTFGRSFKVEEIRCDERYQALKLFTSVFGVGPKTAEKW | Coregonus |
| | YRRGLRSLKEILTEPNIQLNRMQRAGFLYYSDISKAVSKAEAEALGCIIEE | clupeaformis |
| | TVHWIAPDAVLALTGGFRRGKEYGHDVDFLLTVPEMGKEEGLLLHVIDRLR | |
| | DQGILLYCDHQGSTFDVSKLPSCRFEDMDHFQKCFLILRLEEGQVEGERGL | |
| | QRDPGDSRGWRAVRVDLVAPPVDRYAFALLGWTGSKQFGRDLRTFARKERQ | |
| | MLLDNHALYDKTKKLFLPATTEEDIFAHLGLEYVEPWQRNA[YPRAGP/GL] | |
| | TENHNKIFTDVMEELAESSEFNESKGPCLAFRQAASVLKSLPSAVRCLGA | |
| | IQGLP | |
| 107 | CLGEHTKAVMEEILTFGRSFKVEEIRCDERYQALKLFTSVFGVGPKTAEKW | Oncorhynchus |
| | YRRGLRSLQEILTEPNIQLNRMQRAGFLYYSDISKAVSKAEAKALGCIIEE | nerka |
| | TVHWIAPDAILALTGGFRRGKEYGHDVDFLLTMPEMGKEEGLLLHVIDRLK | |
| | DQGILLYCEYQGSTFDVSKLPSCRFEDMDCFQKCFLILRLEQGQVAGERGL | |
| | QRDPGDSRGWRAVRVDLVAPPVDRYAFALLGWTGSKQFGRDLRTFAQKERL | |
| | MLLDNHALYDKTKKLCLLATTEEDIFAHLGLEYVEPWQRNA[YPRAGP/GL] | |
| | TENHNKIFTDVMEELAESSEFNESKGPCLAFRQAASVLKSLPSAVHCLEA | |
| | IQGLP | |
| 108 | CLENKVKCIIEEIIEDGESSEVKAVLNDERYQSFKLFTSVFGVGLKTSEKW | Vicugna |
| | FRMGFRTLSKIRSDETLKFTRMQKAGFLYYEDLVSCVTRAEAEAVSVLVKE | pacos |
| | AVWAFLPDAFVTMTGGFRRGKQIGHDVDFLITSPGTTEAEEEQLLPKVINL | |
| | WERKELLLYCDLVESTFEKSKLPSRKVDALDHFQKCFLILKLHHQRVDSGL | |
| | SRRQEGKTWKAIRVDLVMCPYEHRAFALLGWTGSKQFERDLRRYATHERKM | |
| | ILDNHALYDKTKRIFLKAESEEEIFAHLGLDYIEPWERNA[YPRAGP/GL] | |
| | LNNCNHIFTDAFEVLAENYEFRENEIFSVTFLRAASVLKSLPFTIISMKDT | |
| | EGIP | |
| 109 | CLADKVKCVIEEIIEDGESSEVKAVLNDERYKSFKLFTSVFGVGLKTAEKW | Elephantulus |
| | FRLGFRTLSGIMNDKTLKLTHMQKAGFLYYEDLVSCVTRAEAEAVGVLVKE | edwardii |
| | AVWAFLPDAIVTMTGGFRRGKKVGHDVDFLITSPEATEEQEQQLLHKVITF | |
| | WEKEGLLLYCDLYESTFEKLKMPSRKVDALDHFQKCFLILKLHRECVDDGT | |
| | SSQLQGKTWKAIRVDLVVCPYECRAFALLGWTGSPQFERDLRRYATHERKM | |
| | MLDNHALYDKTKRKFLSADSEEDIFAHLGLDYIEPWERNA[YPRAGP/GL] | |
| | LNNHNHIFTDAFEILAENCEFRENEGSYVTYMRAASVLKSLPFSIISMKDT | |
| | EGIP | |
| 110 | CLGDNVKCVIEEIIEDGESSEVKNVLNDERYQSFKLFTSVFGVGLKTSERW | Galeopterus |
| | FRMGFRTLSTIRSDKSLKFTRMQKAGFLYYEDLVSCVTRAEAEAVSVLVKE | variegatus |
| | AVWAFLPDALVTMTGGFRSQGLLLYHDLVESTFEKLKLPSRNVDALDHFQK | |
| | CFLILKLDHQRVDSDKSNQQEGKTWKAIRVDLVMCPYECRAFALLGWTGSP | |
| | QFERDLRRYATHERKMRLDNHALYDKTKRIFLKAESEEEIFAHLGLDYIEP | |
| | WERNA[YPRAGP/GL]LNNCNHMFTDAFEILAENYEFRENEDSYLAFKRAA | |
| | SVLKSLPFTIISMRDTEGIP | |
| 111 | LIGVEMKYIIEEILEEGQCCRVLEVLNSERYKAFKAFTSIFGVGLKTSEKW | Engystomops |
| | FRMGLRTLEDIKYKKDLKLTTMQKHGLLYYDDIADYVSKMEAEAVEHLMKN | pustulosus |
| | IIYKFVPDAIVTITGGFRRGKELGHDVDLLITCPRKGVEREILHNTLNVLK | |
| | NQGQLLFYDIYESTFDDTKRPSRRVDALDHYQKSFLILKLSRKEVKAEKQR | |
| | DDGWTRLEDLRDWKAVRVDLVITPHEQYAFALLGWTGSPQFERDLRRYATQ | |
| | EKKMMLDNHALFDKTKNVFLKARSEEDIFAHLGLDYVLPEERNA[YPRAGP | |
| | /GL]DAFDILAENFEFEINKSPLVAFRRASSVLKSLQSPIMSTKDLEGLP | |
| 112 | LQDINRIFTDALDTLAENYEFWENKGPLVAFRRASSVLKSLQSPIVSIKDL | Bufo bufo |
| | RGLPLLGDEMKHIIEEILEEGKCWRVLEVVNSERYNAFKVFTSIFGVGLKT | |
| | AEKWHRMGFRTLEDIKTKKDLKLTRMQKHGLLYYEDITSHVSKMEADAVEH | |
| | LVKNIICEFVPDAIVTITGGFRRGKDVGHDVDLLITCPRRGAEKNILHNTI | |
| | NVLKNQGRLLFYDIIESTFDDTKLPSRRVDALDHYQKSFIILKLLKKEVKA | |
| | QIPRDYGWTEPEDTYPRDWKAVRVDLVISPQEQYAFALLGWTGSPQFERDL | |
| | RRYASQEKKMMLDNHALFDKTKHIFMRARTEQDIFDHLGLDYVEPGDRNA | |
| | [YPRAGP/GL]LQDINRIFTDALDTLAENYEFWENKGPLVAFRRASSVLKSL | |
| | QSPIVSIKDLRGLP | |
| 113 | LLGDEMKHIIEEILEEGKCWRVLEVVNSERYKAFKVFTSIFGVGLKTAEKW | Bufo |
| | HRMGFRTLEDIKTKKDLKLTRMQKHGLLYYEDITSHVSKMEADAVEHLVKN | gargarizans |
| | IICEFVPDAIVTITGGFRRGKDVGHDVDLLITCPRRGAEKNILHNTINVLK | |
| | NQGRLLFYDIIESTFDDTKLPSRRVDALDHYQKSFIILKLLKKEVKAQIPE | |
| | DDGWTEPEDTYPRDWKAVRVDLVISPQEQYAFALLGWTGSPQFERDLRRYA | |
| | SQEKKMMLDNHALFDKTKHIFLRARTEQDIFDHLGLDYLEPGDRNA[YPRA | |
| | GP/GL]LQDINRIFTDALDTLAENYEFLENKGPLVAFRRASSVLKSLQSPI | |
| | VSIKDLRGLP | |
| 114 | CLGDKVKCIIEEIIEDGESSEVKAVLNDERYKSFKLFTSVFGVGLKTSERW | Sciurus |
| | FRMGFRSLSKIRSDKSLKFTHMQKAGRGHRTPKHNGKAWEMLRKSQARNAA | carolinensis |
| | GPLRPGSNRVSLCLEFPPGLCAGWEGGNEGGQAGSSRGHGSEPSPTDKPDP | |
| | DGLAQAQRRKPTCTDAEARALVQRDHRAPPCKASKPRRLDWCSLVLLYPFS | |
| | YPFSVDRTCEGLLLYCDLVESTFEKLKIPSRKVDALDHFQKCFLILKLDHQ | |
| | RVDSDKASQQDGKTWKAIRVDLVMCPYERRAFALLGWTGSPQFERDLRRFA | |
| | THERKMVLDNHALYDKTKRIFLKAESEEEIFAHLGLDYIEPWERNA[YPRA | |
| | GP/GL]LDNHNHVFTDAFEVLAENYEFRENEGCYLIFMRAASVLKSLPFTI | |
| | SSMKDIEGIP | |
| 115 | HLGEEMKYIIEEILEDGKSSRVDQVIYSERYKSFKIFTSIFGVGLKTAEKW | Nanorana |
| | YRMGYRTLEDITSNKGLTFTSMQKHGLLYYEDITSSVTKTEADAVEHLIRT | parkeri |
| | VICGFVSDAIVTVTGGFRRGKDVGHDVDILITCPQKRDENQILHKTINALK | |
| | KRDLLLFCYLVESTFDDTKRPSRYIDSLDHFQKCFLILQLQKEESKNAYSH | |
| | KPKGCDAKRSWKAIRVDLVVTPYEQYAFALLGWTGSQQFERDLRRYASHEK | |
| | KMMLDNHAMYDKSKNIFLKAETEEDIFTHLGLEYLEPWERNA[YPRAGP/G | |
| | L]LQDINRIFTDAFDILAENYEFLENKGPRVAYQRASAVLKSLPFPILTMK | |
| | DLEGLP | |
| 116 | CLGEHSRAVIEEIIEYGSSSRVEEILNDERYRTMKLFSSVFGVGPKTSESW | Carassius |
| | FCRGLRSFEQVLNEPSVRLNRMQTAGFLFYEDISKPVSRTEASALKIIVEE | auratus |
| | AVISVNPSATVSITGGFRRGKEFGHDVDFIIKTPEAGQEDAILTAVIERFK | |
| | SQNILLYSDFQESTFDLRQLPTHCFEAMDHYSKCFLIIKLKREQETDARAG | |
| | RDWRAVRVDLVAPPLERFAYALLGWTGSTLFERDLRRFARLERGKLLDNHT | |
| | LYDKATNTFLPANTEEDIFAHLGLEYIEPWQRNA[YPRAGP/GL]MDNHNK | |
| | IFTDALEVLAENLEFGGNQGPSLAFRRAASVLKSLPAALKHLQETQHLP | |
| 117 | CLGEHSKAVLEEIFECGSSSRAEDTLNDERYRTMKLFSSVFGVGPRTAESW | Pimephales |
| | YCRGLRTFEQVLTEPSIRLNRMQTAGFLFYEDISKPVSRTEASALKIIVEE | promelas |
| | AVMCVNPHATVHITGGFRRGKEFGHDVDFIIKTPEPGQENAVLPALIQRFK | |
| | AQNILLYSDFQDSTFDLSQLPTHRFEAMDHFSKCFLIVKLKREQESGEREA | |
| | SRDWRAVRVDLVAPPLERYAYALLGWTGSTLFERDLRRFARLERGKLLDNH | |
| | ALYDKATKAFLPACTEEEIFAHLGLEYIEPWQRNA[YPRAGP/GL]LDNHN | |
| | KIFTDALEVLAENMEFVGNRGPCLAFRRAASVLKSLRAPLQHLEETQRLP | |
| 118 | CLGEQIKSVIEEIFEFRTCSKVEEILRDEKYQTLKLFTSVFGVGPKTAEKW | Chanos |
| | YRKGLRTLKAVLAEPDIHLNKMQEAGFLYYEDISKSVSKADAQAVETLIKD | chanos |
| | TISYYSSDTKLFLTGGFRRGKEHGHDVDFILTTPEIGKEQALLLFLIDKEK | |
| | NLGILLYSDFQESTFDIQKLPSCKFEAMDHFPKCFLILKLKTGEQTAGVQQ | |
| | NQNDSRDWKAIRVDLVAPPADRYAFAMLGWTGSTQFERDLRRYARKERRML | |
| | LDNHALYDKTKHAFLEAQTEEEIFDLLGLEYIEPWQRNA[YPRAGP/GL]L | |
| | DNQNKIFTDAFEVLAESNEFLDSRGQCLAFRRAASVLKSLQFTLRSLDDIE | |
| | GLP | |
| 119 | WMGKQVKGIIEDIIEEGESSKVKAVLDNENYRSVKLFTSVFGVGLKTSDKW | Python |
| | YRMGLRTLEEVKRDKNLKLTRMQKAGFLHYDDLTSCVSKAEADAASLIVQD | bivittatus |
| | VVWKIVPNAIVTIAGGFRRGKQTGHDVDFLITVPGSKQEEEELLHTVIDIW | |
| | KKQELLLYYDLIESTFEDTKLPSRKVDALDHFQKCFAILKVHKEREDKGNS | |
| | IRSKAFSEEEIKDWKAIRVDLVVVPFEQYAFALLGWTGSTQFERDLRRYAT | |
| | HEKKMMLDNHALYDKTKKIFLNAASEEEIFAHLGLDYLEPWERNA[YPRAG | |
| | P/GL]LKNYNQKFTDAFEILAENYEFNENKGFCTAFRRAASVLKCLPFTIV | |
| | QVHDIEGVP | |
| 120 | CLGDNSRAVLEEICECGSSSRVEEIQNDQRYRTMKLFCSVFGVGPKTAESW | Danio rerio |
| | FCRGLKTFEQVLTEPSIRLNRMQTAGFLFYEDISVPVSRAEAAALKMMMEE | |
| | ALLFINPSATVTITGGFRRGKEFGHDVDFIIKAPEGQEDRILPAVIKRFKS | |
| | QNVLLYSDFQKSTFDLRQLPNHRFEAMDRFSKCFLLVKLQMQESRTGRNWR | |
| | AVRVDLVAPPLERFPYALLGWTGSTLFERDLRRFARLERGKLLDNHTLYDK | |
| | TTKTFLPANTEEDIFQHLGLEYIEPWQRNA[YPRAGP/GL]LENHNKIFTE | |
| | ALEVLALNSEFSGNQGSCLGFRRAASVLKSLPAALRSPEEALRLP | |

### Nucleic Acids

The present disclosure also provides nucleic acid molecules encoding modified TdT polymerases (e.g., TdT variants, chimeric TdT variants, circularly permuted TdTs) as provided herein, including subsequences, sequence variants and modified forms of the sequences, and vectors that include nucleic acids that encodes the peptide. As used herein, the phrase “nucleic acids” include those that encode the exemplified peptide sequences disclosed herein, as well as those encoding functional subsequences, sequence variants and modified forms of the exemplified peptide sequences, so long as the foregoing retain at least detectable or measurable activity or function. For example, a subsequence, a variant or modified form of an exemplified peptide sequence disclosed herein that retains some activity associated with TdT and, optionally, the enhanced properties described herein.

As used herein the terms “nucleic acid”, “polynucleotide”, and/or “oligonucleotide” and/or grammatical equivalents thereof can refer to at least two nucleotide monomers linked together as a polymer. The two or nucleotide-containing polymers are typically linked by a phosphodiester bond or analog thereof. The terms can be used interchangeably to refer to all forms of nucleic acid, including deoxyribonucleic acid (DNA) and ribonucleic acid (RNA). The nucleic acids can be single strand, double, or triplex, linear or circular. Nucleic acids include genomic DNA and cDNA. RNA nucleic acid can be spliced or unspliced mRNA. Nucleic acids include naturally occurring, synthetic, as well as nucleotide analogues and derivatives.

As a result of the degeneracy of the genetic code, nucleic acid molecules include sequences degenerate with respect to nucleic acid molecules encoding the illustrative peptide sequences provided herein. Thus, degenerate nucleic acid sequences encoding peptide sequences, including subsequences, variants and modified forms of the peptide sequences exemplified herein (e.g., provided in the Sequence Listing), are provided. As used herein, the term “complementary,” when used in reference to a nucleic acid sequence, means the referenced regions are 100% complementary, i.e., exhibit 100% base pairing with no mismatches.

In some embodiments, nucleic acid sequences encoding modified TdT polymerases (e.g., TdT variants, chimeric TdT variants, circularly permuted TdTs) of the present disclosure include codon-optimized nucleic acid sequences. As used herein, “codon-optimized” refers to a gene coding sequence that has been optimized to improve one or more properties of the polynucleotide. For example, codon optimization can enhance expression by replacing one or more codons normally present in a given coding sequence (e.g., wild-type sequence, including, for example, the dystrophin or mini-dystrophin), a codon for the same amino acid (synonymous). Thus, as described and provided herein, a protein encoded by its corresponding gene is identical as when no codon optimization is present, but the underlying nucleotide sequence of the gene or the corresponding mRNA is different, and thus “optimized.” In some such embodiments, one or In some embodiments, optimization replaces one or more rare codons (i.e., codons for tRNAs that are relatively infrequent in cells of a particular species) with synonymous codons that are more common to improve translation efficiency. For example, in human codon-optimization, one or more codons in the coding sequence are replaced with codons that occur more frequently in human cells for the same amino acid. Codon-optimization can also lead to increased gene expression through other mechanisms that can increase the efficiency of transcription and/or translation. Strategies may include, without limitation, increasing total GC content (i.e., the percentage of guanines and cytosines in the entire coding sequence), decreasing CpG content (i.e., the number of CG or GC dinucleotides in the coding sequence), removing cryptic splice donor or acceptor sites, and/or addition or removal of ribosome entry sites such as Kozak sequences. Preferably, if a codon-optimized gene provides increased expression of its corresponding protein, for example, the protein encoded by it (the codon-optimized gene) is expressed at a significantly higher level in the cell as compared to the level of protein expression achieved with the wild-type gene in an otherwise identical cell.

As will be understood by those of skill in the art, nucleic acids can be produced using any of a variety of known cloning methods, chemical synthesis methods, enzymatic synthesis methods, and can be altered intentionally by site-directed mutagenesis or other recombinant techniques known to one skilled in the art. Purity of polynucleotides can be determined through sequencing, gel electrophoresis, UV spectrometry.

In some embodiments, a polynucleotide of the present disclosure comprises a sequence encoding the TdT polymerase of SEQ ID NO: 1, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more thereto. In some embodiments, the polynucleotide comprises a nucleic acid sequence encoding the TdT polymerase set forth in SEQ ID NO: 1, or a sequence having at least 60%, at least 65%, at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity to SEQ ID NO: 1.

In some embodiments, the polynucleotide comprises or consists of a nucleic acid sequence encoding a cpTdT polymerase comprising or consisting of an amino acid sequence selected from any one of SEQ ID NOs: 88-120 or an amino acid sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or more or 100% identity thereto.

In some embodiments, the polynucleotide comprises or consists of a nucleic acid sequence encoding a modified TdT polymerase (e.g., a TdT variant, a chimeric TdT variant) comprising or consisting of an amino acid sequence selected from any one of SEQ ID NOs: 32-80, or encoding a modified TdT polymerase comprising or consisting of an amino acid sequence having at least 90%, at least 95%, at least 96%, at least 97%, at least 98%, or at least 99% identity thereto.

In some embodiments, the polynucleotide comprises or consists of a sequence encoding the modified TdT polymerase comprising or consisting of the amino acid sequence of SEQ ID NO: 32, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 33, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 34, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 35, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 36, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 37, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 38, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 39, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 40, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 41, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 42, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 43, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 44, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 45, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 46, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 47, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 48, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 49, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 50, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 51, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 52, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 53, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 54, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 55, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 56, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 57, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 58, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 59, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 60, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 61, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 62, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 63, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 64, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 65, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 66, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 67, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 68, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 69, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 70, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 71, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 72, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 73, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 74, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 75, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 76, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 77, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 78, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 79, or a sequence having at least 90%, at least 95%, at least 96%, at least 97%, at least 98%, or at least 99% identity thereto.

In some embodiments, the polynucleotide comprises or consists of a nucleic acid sequence encoding the modified TdT polymerase comprising or consisting of the amino acid sequence of SEQ ID NO: 80, or a sequence having at least 90%, at least 95%, at least 96%, at least 97%, at least 98%, or at least 99% identity thereto.

### Vectors

The present disclosure provides vectors comprising polynucleotides encoding one or more modified TdT polymerases as provided herein. Vectors include, without limitation, plasmids or engineered viral vectors, many formats of which are known to those skilled in the art.

Nucleic acids may be inserted into a plasmid or other vector, e.g. a viral vector, for transformation into a host cell and for subsequent expression and/or genetic manipulation. Vectors maybe also be referred to as a “nucleic acid construct.” In some embodiments, a plasmid is a nucleic acid that can be propagated, stably, or transiently, in a host cell. In some embodiments, plasmids may optionally comprise expression control elements for activating or deactivating expression of the nucleic acid.

As used herein, “expression” may refer to transcription, or to transcription and translation of polynucleotides provided herein. For example, a plasmid or vector may be expressed in vitro using a commercially available cell-free expression system. In some embodiments, plasmids and vectors may contain at least an origin of replication for propagation in a cell and a promoter. In some embodiments, plasmids and vectors may also include an expression control element for expression in a host cell. In some such embodiments, it is contemplated that such plasmids and/or vectors may be useful for expression and/or genetic manipulation of nucleic acids encoding peptide sequences, expressing peptide sequences in host cells and organisms, or producing a polypeptide sequence, e.g. of a modified TdT polymerase as provided herein.

As is understood by those of skill in the art, nucleic acids may be inserted into a nucleic acid construct in which expression of the nucleic acid is influenced or regulated by an “expression control element.” In some embodiments of the present disclosure, a vector comprising an expression control element and one or more modified TdT polymerases (e.g., TdT variants, chimeric TdT variants, circularly permuted TdTs) provided herein may be referred to as an “expression cassette.” As used herein, “expression control element” refers to one or more nucleic acid sequence elements that regulate or influence expression of a nucleic acid sequence to which it is operatively linked. An expression control element can include, as appropriate, promoters, enhancers, transcription terminators, gene silencers, a start codon (e.g., ATG) in front of a protein-encoding gene, etc.

In some embodiments, an expression control element operatively linked to a nucleic acid sequence can control transcription and, as appropriate, translation of the nucleic acid sequence. The term “operatively linked” refers to a juxtaposition wherein referenced components are in a relationship permitting them to function in their intended manner. Typically, expression control elements are juxtaposed at the 5′ or the 3′ ends of the genes but can also be intronic.

Expression control elements can include elements that activate transcription constitutively, that are inducible (i.e., require an external signal or stimuli for activation), or derepressible (i.e., require a signal to turn transcription off; when the signal is no longer present, transcription is activated or “derepressed”). Also provided in expression cassettes in accordance with the present disclosure are control elements sufficient to render gene expression controllable for specific cell-types or tissues (i.e., tissue-specific control elements). Typically, such elements are located upstream or downstream (i.e., 5′ and 3′) of the sequence encoding any modified TdTs as provided herein. In some embodiments, promoters can be positioned 5′ of the coding sequence. In some embodiments, promoters, produced by recombinant DNA or synthetic techniques, can be used to provide for transcription of the polynucleotides encoding modified TdT polymerases in accordance with the present disclosure.

A “promoter” typically refers to a minimal sequence element sufficient to direct transcription. By way of non-limiting example, bacterial system promoters include T7 and inducible promoters such as pL of bacteriophage k, plac, ptrp, ptac (ptrp-lac hybrid promoter) and tetracycline responsive promoters. To give but one example of an inducible promoter, a promoter commonly used in E. coli is the lac promoter, which can be activated by the addition of isopropyl-β-D-thiogalactoside (IPTG). Insect cell system promoters can include constitutive or inducible promoters (e.g., ecdysone). Mammalian cell constitutive promoters can include, among others, SV40, RSV, bovine papilloma virus (BPV) and other virus promoters. In some embodiments, mammalian cells can include inducible promoters derived from a mammalian cell genome (e.g., metallothionein IIA promoter; heat shock promoter) or from mammalian viruses (e.g., the adenovirus late promoter; the inducible mouse mammary tumor virus long terminal repeat). Alternatively, a retroviral genome can be genetically modified for introducing and directing expression of a peptide sequence in appropriate host cells.

As is well-understood in the art, vectors can include constitutive and inducible promoters (see, e.g., Ausubel et al., In: Current Protocols in Molecular Biology, Vol. 2, Ch. 13, ed., Greene Publish. Assoc. & Wiley Interscience, 1988; Grant et al. Methods in Enzymology, 153:516 (1987), eds. Wu & Grossman; Bitter Methods in Enzymology, 152:673 (1987), eds. Berger & Kimmel, Acad. Press, N.Y.; and, Strathern et al., The Molecular Biology of the Yeast Saccharomyces (1982) eds. Cold Spring Harbor Press, Vols. I and II). A constitutive promoter such as ADH or LEU2 or an inducible promoter such as GAL may be used (R. Rothstein In: DNA Cloning, A Practical Approach, Vol. 11, Ch. 3, ed. D. M. Glover, IRL Press, Wash., D.C., 1986). For example, vectors that facilitate integration of foreign nucleic acid sequences into a chromosome, via homologous recombination, are known in the art. For instance, yeast artificial chromosomes (YAC) are typically used when inserted polynucleotides are too large for more conventional vectors (e.g., greater than about 12 Kb).

Expression vectors also can contain a selectable marker that confers certain resistance to one or more selective pressures and/or includes an identifiable marker (e.g., beta-galactosidase), thereby allowing cells having the vector to be identified/selected for, grown, and expanded. Alternatively, a selectable marker can be on a second vector that is co-transfected into a host cell with a first vector that comprises a nucleic acid encoding a polypeptide sequence. Selection systems include but are not limited to herpes simplex virus thymidine kinase gene (Wigler et al., Cell 11:223 (1977)), hypoxanthine-guanine phosphoribosyltransferase gene (Szybalska et al., Proc. Natl. Acad. Sci. USA 48:2026 (1962)), and adenine phosphoribosyltransferase (Lowy et al., Cell 22:817 (1980)) genes that can be employed in tk-, hgprt- or aprt-cells, respectively. Additionally, antimetabolite resistance can be used as the basis of selection for dhfr, which confers resistance to methotrexate (O'Hare et al., Proc. Natl. Acad. Sci. USA 78:1527 (1981)); the gpt gene, which confers resistance to mycophenolic acid (Mulligan et al., Proc. Natl. Acad. Sci. USA 78:2072 (1981)); neomycin gene, which confers resistance to aminoglycoside G-418 (Colberre-Garapin et al., J. Mol. Biol. 150:1(1981)); puromycin; and hygromycin gene, which confers resistance to hygromycin (Santerre et al., Gene 30:147 (1984)). Additional selectable genes include trpB, which allows cells to utilize indole in place of tryptophan; hisD, which allows cells to utilize histinol in place of histidine (Hartman et al., Proc. Natl. Acad. Sci. USA 85:8047 (1988)); and ODC (ornithine decarboxylase), which confers resistance to the ornithine decarboxylase inhibitor, 2-(difluoromethyl)-DL-ornithine, DFMO (McConlogue (1987) In: Current Communications in Molecular Biology, Cold Spring Harbor Laboratory).

In some embodiments, a vector comprises a nucleic acid sequence in accordance with the present disclosure. In some embodiments, the vector comprises a nucleic acid sequence encoding the TdT polymerase of SEQ ID NO: 1, or a modified TdT polymerase (e.g., a TdT variant, e.g., a chimeric TdT variant) sequence having at least 90%, at least 95%, at least 96%, at least 97%, at least 98%, or at least 99% thereto. In some embodiments, the vector comprises a nucleic acid sequence encoding the TdT polymerase of SEQ ID NO: 1, or a modified TdT polymerase (e.g., a TdT variant, e.g., a chimeric TdT variant) sequence having at least 60%, at least 65%, at least 70%, at least 75%, at least 80%, at least 85%, at least 90%, at least 95%, at least 96%, at least 97%, at least 98%, or at least 99% identity to SEQ ID NO: 1.

In some embodiments, the vector comprises a nucleic acid sequence encoding a cpTdT comprising an amino acid sequence selected from any one of SEQ ID NOs: 88-120, or an amino acid sequence having at least 90%, at least 95%, at least 96%, at least 97%, at least 98%, or at least 99% identity thereto.

In some embodiments, the vector comprises a nucleic acid sequence encoding a modified TdT comprising an amino acid sequence selected from any one of SEQ ID NOs: 32-80, or an amino acid sequence having at least 90%, at least 95%, at least 96%, at least 97%, at least 98%, or at least 99% identity thereto.

### Host Cells

The disclosure provides host cells comprising nucleic acids and/or vectors as provided herein. As used herein, “host cell” includes an individual cell or cell culture that can be or has been a recipient of one or more nucleic acids and/or vectors as provided herein. Host cells include progeny of a single host cell. In some embodiments, progeny may not necessarily be completely identical (e.g., in morphology or in genomic of total DNA complement) to the original parent cell due to natural, accidental, or deliberate changes (e.g., mutation). In some embodiments, a host cell provided by the present disclosure includes cells transfected in vivo with a nucleic acid and/or vector as provided herein.

In some embodiments, a host cell is a transformed cell(s) (in vitro, ex vivo and in vivo) and host cells that produce the modified TdTs (i.e., TdT variants) described herein, where expression of the TdT variant is conferred by a vector comprising a nucleic acid encoding the TdT variant. Transformed host cells that express nucleic acid sequences or vectoring comprising a nucleic acid encoding the TdT variant can include a nucleic acid that encodes the TdT variants provided herein. In some embodiments, a transformed or host cell is a prokaryotic cell. In some embodiments, the prokaryotic cell is a bacterial cell. In some embodiments, the bacterial cell is an E. Coli cell. In another embodiment, a transformed or host cell is a eukaryotic cell. In various aspects, the eukaryotic cell is a yeast or mammalian (e.g., human, primate, etc.) cell.

As used herein, a “transformed” or “host” cell is a cell into which a vector or nucleic acid is introduced that can be propagated and/or transcribed for expression of an encoded peptide sequence. The term also includes any progeny or subclones of the host cell.

Any suitable cell type for recombinant protein expression can be a host cell. Transformed and host cells include but are not limited to microorganisms such as bacteria and yeast; and plant, insect and mammalian cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal, e.g. mammalian, cell systems infected with recombinant virus expression vectors (e.g., retroviruses, adenovirus, vaccinia virus), or transformed animal cell systems engineered for transient or stable propagation or expression.

In some embodiments, the host cell comprises a vector or a nucleic acid sequence/polynucleotide as provided herein. In some embodiments, the vector or polynucleotide comprises or consists of a nucleic acid sequence encoding the TdT set forth in SEQ ID NO: 1, or a modified TdT polymerase (e.g., a TdT variant, a chimeric TdT variant, a circularly permuted TdT variant) comprising or consisting of an amino acid sequence having at least 90%, at least 95%, at least 96%, at least 97%, at least 98%, or at least 99% thereto.

In some embodiments, the vector or polynucleotide comprises or consists of a nucleic acid comprising a sequence encoding the TdT set forth in SEQ ID NO: 1, or a modified TdT polymerase (e.g., a TdT variant, e.g., a chimeric TdT variant) comprising or consisting of an amino acid sequence having at least 60%, at least 65%, at least 70%, at least 75%, at least 80%, at least 85%, at least 90%, at least 95%, at least 96%, at least 97%, at least 98%, or at least 99% identity to that of SEQ ID NO: 1.

In some embodiments, the vector or polynucleotide comprises or consists of a nucleic acid sequence encoding a modified TdT polymerase comprising or consisting of an amino acid sequence selected from any one of SEQ ID NOs: 32-80, or a sequence having at least 90%, at least 95%, at least 96%, at least 97%, at least 98%, or at least 99% identity thereto.

In some embodiments, the vector or polynucleotide comprises or consists of a nucleic acid sequence encoding a circularly permuted TdT variant comprising or consisting of an amino acid sequence selected from any one of SEQ ID NOs: 88-120, or a sequence having at least 90%, at least 95%, at least 96%, at least 97%, at least 98%, or at least 99% identity thereto.

### Methods of Producing Modified TdT Polymerases

The present disclosure also provides methods of producing modified TdT polymerases (e.g., TdT variants, chimeric TdT variants, circularly permuted TdTs) as provided herein. In some embodiments, a method of producing a modified TdT polymerase in accordance with the present disclosure comprises culturing a host cell under appropriate conditions for expressing a modified TdT polymerase. In some embodiments, a method of producing comprises isolating a modified TdT polymerase such as from a culture or population of cultured host cells. Methods of isolating polymerases from cultured cells are generally known to those of skill in the art.

As used herein, the term “cultured,” when used in reference to a host cell, means that the cell is grown in vitro. A particular example of such a cell is a host cell compatible with any of the vectors and/or polynucleotides provided herein. To give but one non-limiting example, a bacterial cell (e.g., an E. coli cell) may serve as a host cell. For example, in some embodiments, a bacterial cell (e.g., an E. coli cell) may comprise a vector or polynucleotide, which comprises or consists of a sequence encoding a modified TdT polymerase as provided herein.

As used herein, the terms, “isolated” or “purified” are used to describe one or more modified TdT polymerases that have been identified and separated and/or recovered from a component of the environment in which it was expressed (e.g., host cell components and culture environment). For example, contaminant components of an environment in which a modified TdT polymerase is or was expressed can include materials that would typically be expected to interfere with certain methods of using a modified TdT polymerase (e.g., in an extension reaction) and may include, among other things, enzymes, hormones, other proteinaceous or non-proteinaceous solutes, insoluble debris, host cell genomic nucleic acids, and the like. In general, a modified TdT polymerase made by recombinant means known to those in the art, expressed in a host cell and/or removed from a host cell and any culture components after expression is considered “separated” (e.g., from other components), “isolated,” or “purified.”

Any suitable method of isolating proteins can be applied to separating, isolating or purifying modified TdT polymerases of the present disclosure. A number of chromatography methods are known in the art, and many techniques and protocols are available from commercial vendors to use such methods for separating a modified TdT polymerase from other components (e.g., hot cell components, etc.). Chromatography methods can include, for example, ion exchange/anion exchange chromatography such as DE52 or Q resins, affinity chromatography (e.g. an IMAC column such as Ni-NTA), and desalting chromatography (e.g. a gel filtration column such as Sephadex G25). Such methods may comprise, among other things, chromatography resins, membranes, columns, and the like. Alternatively or additionally, as will be known to those in the art, other methods exist for isolation (e.g., further isolation/purification) such as dialysis and size selection filters.

### Conjugates

The present disclosure provides conjugates comprising modified TdT polymerases (e.g., TdT variants, chimeric TdT variants, circularly permuted TdTs) as provided herein. Such conjugates comprise a TdT polymerase and a nucleotide (e.g. a synthetic nucleotide). In some embodiments, the nucleotide is a nucleotide analog. In some embodiments, the nucleotide is attached to a modified TdT polymerase via a linker. In some embodiments, the linker is selectively cleavable.

In some embodiments, conjugates provided herein further comprise one or more additional moieties that sterically hinder a tethered nucleoside triphosphate (or a tethered nucleic acid post-elongation) from approaching catalytic sites of another conjugate molecule (e.g., with the same or different type of modified TdT polymerase and/or the same or different type of nucleotide). Such moieties include, for instance, polypeptides or protein domains that can be inserted into a loop of a given modified TdT polymerase, and those and other bulky molecules such as polymers that can be site-specifically ligated e.g. to an inserted unnatural amino acid or specific polypeptide tag.

### Linker

In a conjugate of the present disclosure, a linker comprises atoms that connect the nucleotide of the conjugate to the modified TdT polymerase (e.g., TdT variants, chimeric TdT variants, circularly permuted TdTs) of the conjugate. The linker can attach to the base, the sugar, or a phosphate of the nucleotide or modified nucleotide. In some embodiments, the modified TdT polymerase and the nucleotide are covalently linked and the distance between the linked atom of the nucleotide and the modified TdT polymerase to which it is attached can be, for example, in the range of about 4-100 Å, about 15-40 Å or about 20-30 Å, or a distance appropriate for the position on the modified TdT polymerase to which the nucleotide or nucleotide analog is tethered.

Any suitable linker for tethering the nucleotide or modified nucleotide to the modified TdT polymerase is contemplated in the methods described herein. In some embodiments, the linker comprises a polyether or a polyethylene glycol (PEG). In some embodiments, the linker comprises one or more peptide bonds. In some embodiments, the linker comprises one or more sarcosines. In some embodiments, the linker comprises one or more glycines. In some embodiments, the linker comprises one or more prolines. In some embodiments, the linker comprises a carbamate. In some embodiments, the linker comprises an ester. In some embodiments, the linker joins to the nucleotide at an atom of the nucleobase that is not involved in base pairing. In some embodiments, the linker joins to the nucleotide at an atom of the nucleobase that is involved in base pairing. In such embodiments, the linker is considered to be at least the atoms that connect the modified TdT polymerase to any atom in the monocyclic or polycyclic ring system bonded to the F position of the sugar (e.g., pyrimidine or purine or 7-deazapurine or 8-aza-7-deazapurine). In some embodiments, the linker joins to the nucleotide at an atom of the nucleobase that is involved in base pairing. In some embodiments, the linker is joined to the sugar or to a phosphate of the nucleotide. In some embodiments, the linker is sufficiently long to allow the nucleotide to access the active site of the modified TdT polymerase to which it is tethered. As described in greater detail herein, the modified TdT polymerase of a conjugate is capable of catalyzing the addition of the nucleotide to which it is linked onto the 3′ end of a nucleic acid.

The linker may be attached to any suitable atom on the nucleotide. In some embodiments, the linker is attached to the 5 position of pyrimidines or the 7 position of 7-deazapurines. In other embodiments, the linker may be attached to an exocyclic amine of a nucleobase, e.g. by N-alkylating the exocyclic amine of cytosine with a nitrobenzyl moiety as discussed below. In other embodiments, the linker may be attached to any other atom in the nucleobase, sugar, or a phosphate, as will be apparent to those skilled in the art.

Certain polymerases have a high tolerance for modification of certain parts of a nucleotide, e.g. modifications of the 5 position of pyrimidines and the 7 position of purines are well-tolerated by some polymerases (He and Seela., Nucleic Acids Research 30.24 (2002): 5485-5496.; or Hottin et al., Chemistry. 2017 Feb. 10; 23(9):2109-2118). In some embodiments, the linker is attached to these positions.

In some examples, the conjugate is prepared by first synthesizing an intermediate compound comprising a linker and a nucleotide (referred to herein as a “linker-nucleotide”), and then this intermediate compound is attached to the modified TdT polymerase. In some non-limiting examples, nucleosides with substitutions compared to natural nucleosides, e.g. pyrimidines with 5-hydroxymethyl or 5-propargylamino substituents, or 7-deazapurines with 7-hydroxymethyl or 7-propargylamino substituents may be useful starting materials for preparing linker-nucleotides. An exemplary set of nucleosides with 5- and 7-hydroxymethyl substituents that may be useful for preparing linker-nucleotides is shown below:

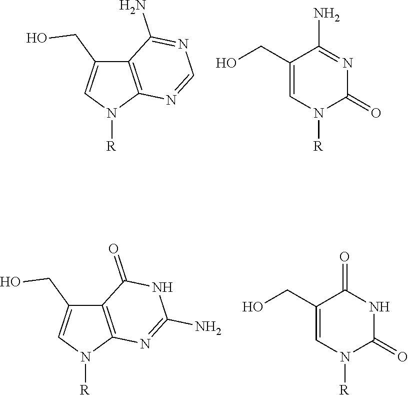

An exemplary set of nucleosides with 5- and 7-deaza-7-propargylamino substituents that may be useful for preparing linker-nucleotides is shown below:

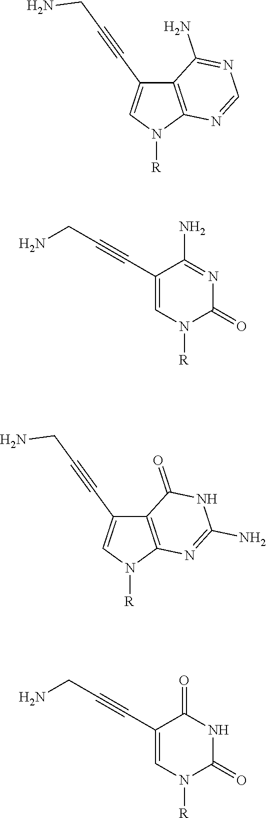

These and other such nucleosides are also commercially available as deoxyribonucleoside triphosphates.

In some embodiments, a conjugate of the present disclosure comprises a pair comprising a linker and a nucleotide or a “linker-nucleotide.” Any suitable nucleotide may be used. In some embodiments, the linker-nucleotide comprises a nucleotide polyphosphate or a modified nucleotide polyphosphate. In some embodiments, the linker-nucleotide comprises a nucleotide triphosphate or a modified nucleotide triphosphate. In some embodiments, the linker-nucleotide comprises a nucleotide tetraphosphate or a modified nucleotide tetraphosphate. In some embodiments, the linker-nucleotide comprises a nucleotide pentaphosphate or a modified nucleotide pentaphosphate. In some embodiments, the linker-nucleotide comprises a nucleotide hexaphosphate or a modified nucleotide pentaphosphate. In some embodiments, the linker-nucleotide comprises a modified nucleobase. In some embodiments, the linker-nucleotide comprises a modified nucleobase. In some embodiments, the modified nucleobase comprises an O- or N-linked modification. In some embodiments, the O- or N-linked modification is removable following incorporation of the nucleotide portion of the linker-nucleotide into a polynucleotide. In some embodiments, the O- or N-linked modification is removable by a photolytic process. In some embodiments, the photolytic process comprises exposure to UV light, wherein the UV light comprises wavelengths at 365 nm and/or 405 nm. In some embodiments, the O- or N-linked modification is removable by a chemical process. In some embodiments, the chemical process is selected from a beta-elimination reaction, a Pd-catalyzed deallylation, and a reduction reaction. In some embodiments, the O- or N-linked modification is removable by an enzymatic process. In some embodiments, the enzymatic process comprises removal by an alkyltransferase or methyltransferase.

In some embodiments, the O- or N-linked modification reduces or eliminates Watson-Crick base pairing in a polynucleotide comprising the modified nucleobase. In some embodiments, the O- or N-linked modification reduces or eliminates secondary structure in a polynucleotide comprising the modified nucleobase. In some embodiments, following removal of the O- or N-linked modification the modified nucleobase comprises a natural nucleobase. In some embodiments, the natural nucleobase is guanine, cytosine, adenine, thymine, or uracil.

### Attachment of a Linker to a Modified TdT Polymerase

As provided herein, in some embodiments, conjugates provided herein comprise a modified TdT polymerase in accordance with the present disclosure tethered to a nucleotide via a linker. In some embodiments, the modified TdT polymerase comprises or consists of a sequence (e.g., a nucleic acid sequence, e.g., an amino acid sequence) corresponding to an amino acid having at least 90%, at least 95%, at least 96%, at least 97%, at least 98%, or at least 99% identity to the amino acid sequence of the TdT of SEQ ID NO: 1. In some embodiments, the modified TdT polymerase comprises or consists of a sequence (e.g., a nucleic acid sequence, e.g., an amino acid sequence) corresponding to an amino acid having at least 60%, at least 65%, at least 70%, at least 75%, at least 80%, at least 85%, at least 90%, at least 95%, at least 96%, at least 97%, at least 98%, at least 99%, or at least 99.1%, at least 99.2%, at least 99.3%, at least 99.4%, at least 99.5%, at least 99.6%, at least 99.7%, at least 99.8%, or at least 99.9% or more identity to SEQ ID NO: 1.

In some embodiments, the TdT comprises or consists of an amino acid sequence of SEQ ID NO: 1.

In some embodiments, the modified TdT polymerase comprises or consists of one or more amino acid substitutions relative to the amino acid sequence of SEQ ID NO: 1. In some embodiments, the modified TdT polymerase comprises or consists of sequence selected from any one of SEQ ID NOs: 32-80, or a sequence having at least 90%, at least 95%, at least 96%, at least 97%, at least 98%, at least 99%, or at least 99.1%, at least 99.2%, at least 99.3%, at least 99.4%, at least 99.5%, at least 99.6%, at least 99.7%, at least 99.8%, or at least 99.9% or more identity thereto. In some embodiments, the modified TdT polymerase is a cpTdT polymerase and comprises or consists of one or more amino acid substitutions relative to the amino acid sequence of SEQ ID NO: 1. In some embodiments, the cpTdT polymerase comprises or consists of sequence selected from any one of SEQ ID NOs: 88-120, or a sequence having at least 90%, at least 95%, at least 96%, at least 97%, at least 98%, at least 99%, or at least 99.1%, at least 99.2%, at least 99.3%, at least 99.4%, at least 99.5%, at least 99.6%, at least 99.7%, at least 99.8%, or at least 99.9% or more identity thereto.

Any suitable linker for tethering a nucleotide to a modified TdT polymerase is contemplated for use in accordance with the present disclosure. In some embodiments, the linker is specifically attached to a cysteine residue of the TdT using a sulfhydryl-specific attachment chemistry. Illustrative sulfhydryl specific attachment chemistries include, without limitation, ortho-pyridyl disulfide (OPSS), maleimide functionalities, 3-arylpropiolonitrile functionalities, allenamide functionalities, haloacetyl functionalities such as iodoacetyl or bromoacetyl, alkyl halides or perfluroaryl groups that can favorably react with sulfhydryls surrounded by a specific amino acid sequence (Zhang, Chi, et al. Nature chemistry 8, (2015) 120-128.). Other attachment chemistries for specific labeling of cysteine residues will be apparent to those skilled in the art or are described in the pertinent literature and texts (e.g., Kim, Younggyu, et al, Bioconjugate chemistry 19.3 (2008): 786-791.).

Attachment of the linker to the modified TdT polymerase can be at a cysteine residue in the modified TdT polymerase amino acid sequence. In some embodiments, the modified TdT polymerase has a single cysteine. For example, in some embodiments, if an alignment is done between a cpTdT and SEQ ID NO: 1, a cysteine position can be determined at a particular position corresponding a position relative to that of SEQ ID NO: 1. For example, in some embodiments, the cysteine is at amino acid position 52 relative to SEQ ID NO: 1. In some embodiments, the cysteine is at amino acid position 54 relative to SEQ ID NO: 1. In some embodiments, the cysteine is at amino acid position 58 relative to SEQ ID NO: 1. In some embodiments, the cysteine is at amino acid position 61 relative to SEQ ID NO: 1. In some embodiments, the cysteine is at amino acid position 62 relative to SEQ ID NO: 1. In some embodiments, the cysteine is at amino acid position 69 relative to SEQ ID NO: 1. In some embodiments, the cysteine is at amino acid position 70 relative to SEQ ID NO: 1. In some embodiments, the cysteine is at amino acid position 73 relative to SEQ ID NO: 1. In some embodiments, the cysteine is at amino acid position 76 relative to SEQ ID NO: 1. In some embodiments, the cysteine is at amino acid position 131 relative to SEQ ID NO: 1. In some embodiments, the cysteine is at amino acid position 134 relative to SEQ ID NO: 1. In some embodiments, the cysteine is at amino acid position 141 relative to SEQ ID NO: 1. In some embodiments, the cysteine is at amino acid position 217 relative to SEQ ID NO: 1. In some embodiments, the cysteine is at amino acid position 277 relative to SEQ ID NO: 1. In some embodiments, the cysteine is at amino acid position 278 relative to SEQ ID NO: 1. In some embodiments, the cysteine is at amino acid position 335 relative to SEQ ID NO: 1. In some embodiments, the cysteine is at amino acid position 339 relative to SEQ ID NO: 1. In some embodiments, the cysteine is at amino acid position 342 relative to SEQ ID NO: 1.

In some embodiments, the linker is attached to a lysine residue via an amine-reactive functionality (e.g. NHS esters, Sulfo-NHS esters, tetra- or pentafluorophenyl esters, isothiocyanates, sulfonyl chlorides, etc.). In some embodiments, the linker is attached to the TdT variant via attachment to a genetically inserted unnatural amino acid, e.g. p-propargyloxyphenylalanine or p-azidophenylalanine that could undergo azide-alkyne Huisgen cycloaddition, though many suitable unnatural amino acids suitable for site-specific labeling exist and can be found in the literature (e.g. as described in Lang and Chin., Chemical reviews 114.9 (2014): 4764-4806.).

In some embodiments, the linker may be specifically attached to the N-terminus of the modified TdT polymerase. In some embodiments, the modified TdT polymerase is mutated to have an N-terminal serine or threonine residue, which may be specifically oxidized to generate an N-terminal aldehyde for subsequent coupling to e.g., a hydrazide. In some embodiments, the modified TdT polymerase is mutated to have an N-terminal cysteine residue that can be specifically labeled with an aldehyde to form a thiazolidine. In some embodiments, an N-terminal cysteine residue can be labeled with a peptide linker via Native Chemical Ligation (NCL).

In some embodiments, a peptide tag sequence may be inserted into the modified TdT polymerase that can be specifically labeled with a synthetic group by an enzyme, e.g. as demonstrated in the literature using biotin ligase, transglutaminase, lipoic acid ligase, bacterial sortase and phosphopantetheinyl transferase (e.g. as described in refs. 74-78 of Stephanopoulos & Francis Nat. Chem. Biol. 7, (2011) 876-884).

In some embodiments, the linker is attached to a labeling domain fused to the modified TdT polymerase. For example, a linker with a corresponding reactive moiety may be used to covalently label SNAP tags, CLIP tags, HaloTags and acyl carrier protein domains (e.g., as described in refs. 79-82 of Stephanopoulos & Francis Nat. Chem. Biol. 7, (2011) 876-884).

In some embodiments, the linker is attached to an aldehyde specifically generated within the modified TdT polymerase, as described in Carrico et al. (Nat. Chem. Biol. 3, (2007) 321-322). For example, after insertion of an amino acid sequence that is recognized by the enzyme formylglycine-generating enzyme (FGE) into the modified TdT polymerase, it may be exposed to FGE, which will specifically convert a cysteine residue in the recognition sequence to formylglycine (i.e., producing an aldehyde). This aldehyde may then be specifically labeled with e.g., a hydrazide or aminooxy moiety of a linker.

In some embodiments, a linker may be attached to the modified TdT polymerase via non-covalent binding of a moiety of the linker to a moiety fused to the modified TdT polymerase. Examples of such attachment strategies include fusing a modified TdT polymerase to streptavidin that can bind a biotin moiety of a linker, or fusing a modified TdT polymerase to anti-digoxigenin that can bind a digoxigenin moiety of a linker. In some embodiments, site-specific labeling may lead to an attachment of the linker to the modified TdT polymerase that may readily be reversed (e.g. an ortho-pyridyl disulfide (OPSS) group that forms a disulfide bond with a cysteine that can be cleaved using reducing agents, e.g. using TCEP), other attachment chemistries will produce permanent attachments.

In some embodiments, the modified TdT polymerase is mutated to ensure specific attachment of the tethered nucleotide to a particular location of the modified TdT polymerase, as will be apparent to those skilled in the art. For example, with sulfhydryl-specific attachment chemistries such as maleimides or ortho-pyridyl disulfides, accessible cysteine residues in a reference TdT (e.g., that of SEQ ID NO: 1, e.g., wild-type TdT) may be mutated to a non-cysteine residue to prevent labeling at those positions. On this “reactive cysteine-free” background, a cysteine residue may be introduced by mutation at the desired attachment position. These mutations preferentially do not interfere with the activity of the modified TdT polymerase.

Other strategies for site-specific attachment of synthetic groups to proteins will be apparent to those skilled in the art and are reviewed in literature, (e.g. Stephanopoulos & Francis Nat. Chem. Biol. 7, (2011) 876-884).

### Cleavage of Linker

As provided herein, a linker may be attached to various positions on a nucleotide in a conjugate and/or a linker-nucleotide of the present disclosure, and a variety of cleavage strategies may be used to cleave a linker. It is understood that the cleavage strategy will be determined by the type of linker joining a nucleotide or modified nucleotide and a TdT polymerase (e.g., of SEQ ID NO: 1, e.g., a modified TdT polymerase, etc.). Any suitable method for cleaving the linker is contemplated.

In some embodiments, the linker is cleaved, wherein following cleavage of the linker a nucleobase comprising a chemical group (i.e. a scar) is formed. The chemical modification (i.e. scar) can be atoms or group of atoms N-linked or O-linked to the nucleobase that are in addition to a natural nucleobase (e.g. adenine, thymine, guanosine, cytosine, or uracil). Illustrative, non-limiting, chemical groups (i.e. scars) following linker cleavage are shown below. In some embodiments, the chemical group prevents the nucleobase from base-pairing (e.g. Watson-Crick base-pairing). In some embodiments, the chemical group is removed by a chemical, photolytic, or enzymatic process. In some embodiments, the nucleobase is capable of base-pairing (e.g. Watson-Crick base-paring) following removal of the chemical group.

In some embodiments, the linker may be cleaved by exposure to any suitable reducing agent such as dithiothreitol (DTT), 3-mercaptoethanol, or tris(2-carboxyethyl)phosphine (TCEP). For example, a linker comprising a 4-(disulfaneyl)butanoyloxy-methyl group attached to the 5 position of a pyrimidine or the 7 position of a 7-deazapurine may be cleaved by reducing agents (e.g. DTT) to produce a 4-mercaptobutanoyloxymethyl scar on the nucleobase. This scar may undergo intramolecular thiolactonization to eliminate a 2-oxothiolane, leaving a smaller hydroxymethyl scar on the nucleobase. An example of such a linker attached to the 5 position of cytosine is depicted below, but the strategy is applicable to any suitable nucleobase:

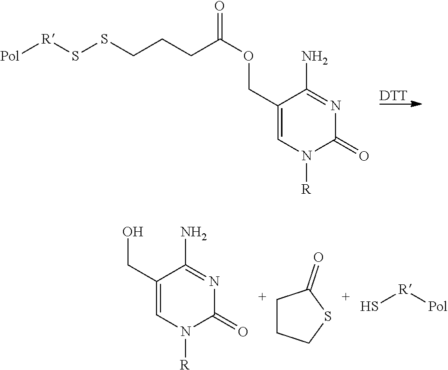

In other embodiments, the linker may be cleaved by exposure to light. For example a linker comprising (2-nitrobenzyl)oxymethyl group may be cleaved with 365 nm light, leaving a hydroxymethyl scar, e.g. as depicted for cytosine below, but as is applicable to any suitable nucleobase:

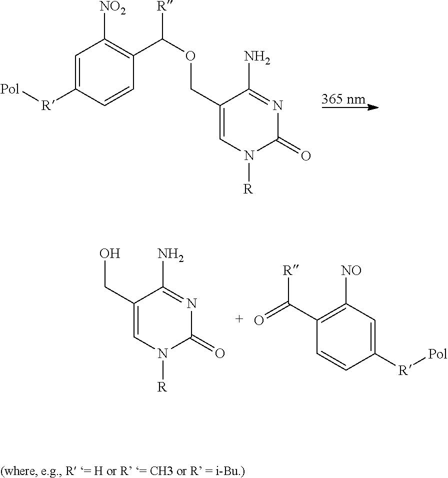

In other embodiments, the linker may comprise a 3-(((2-nitrobenzyl)oxy)carbonyl)aminopropynyl group that may be cleaved with 365 nm light release a nucleobase with a propargylamino scar. This strategy is applicable to any suitable nucleobase:

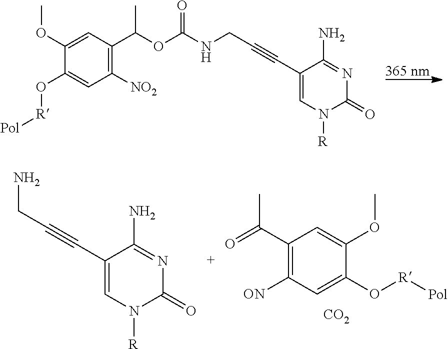

In some embodiments, the linker may comprise an acyloxymethyl group that may be cleaved with a suitable esterase to release a nucleobase with a hydroxymethyl scar, e.g. as depicted for cytosine below, but as is applicable to any suitable nucleobase:

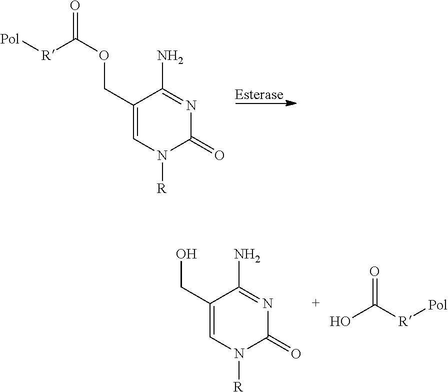

In some such embodiments, the linker may comprise additional atoms (included in R′ above) adjacent to the ester that increase the activity of the esterase towards the ester bond.

In other embodiments, the linker may comprise an N-acyl-aminopropynyl group that may be cleaved with a peptidase to release a nucleobase with propargylamino scar, e.g. as depicted for 5-propargylamino cytosine below, but as is applicable to any suitable nucleobase:

In some such embodiments, the linker may comprise additional atoms (included in R′ above) adjacent to the amide that increase the activity of the peptidase towards the amide

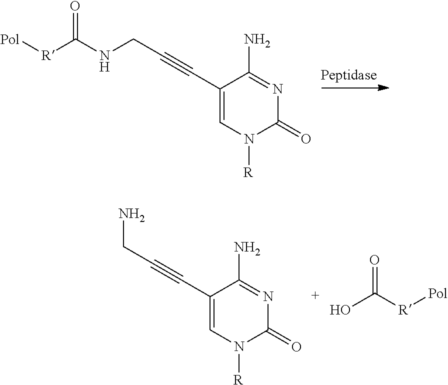

bond.

### Functional Properties of Modified TdT Polymerases or Compositions Thereof

In some embodiments, a modified TdT polymerase (e.g., TdT variant, chimeric TdT variant, circularly permuted TdT) or composition comprising a modified TdT polymerase (e.g., a conjugate) disclosed herein has one or more differences in structural (e.g., sequence) and/or functional properties as compared to those of the TdT polymerase of SEQ ID NO: 1. In some embodiments, a modified TdT polymerase or composition comprising a modified TdT polymerase as provided herein has one or more improved functional properties compared to a TdT polymerase of SEQ ID NO: 1 or conjugate comprising a TdT polymerase of SEQ ID NO: 1. In some embodiments, a cpTdT has one or more improved functional properties compared to a parental TdT polymerase (e.g., a wild type TdT, a modified TdT, a chimeric TdT). Non-limiting examples of functional properties include: increased thermostability, increased nucleotidyl transferase activity, and increased protease resistance. In some embodiments, activity of a modified TdT polymerase or composition comprising the modified TdT polymerase is assessed during nucleic acid synthesis. In some such embodiments, activity is assessed using a fluorescent assay and detected by a fluorometer. For example, in some embodiments, in some embodiments, an assay uses fluorescently detectable/visualizable measurements in where increased fluorescence is representative of nucleotide incorporation onto the initiator oligonucleotide catalyzed by a modified TdT polymerase as provided herein, and is representative of activity (i.e., extent of nucleotide incorporation) of the modified TdT polymerase.

In some embodiments, activity of a modified TdT polymerase or composition comprising the modified TdT polymerase may be assessed at one or more temperatures (e.g. a range of temperatures). In some embodiments, activity of a modified TdT polymerase or composition comprising the modified TdT polymerase is assessed at 37° C., 38° C., 40° C., 44° C., 48° C., 52° C., 54° C., and/or 55° C.

In some embodiments, activity of a modified TdT polymerase or composition comprising the modified TdT polymerase as provided herein has increased thermostability (e.g., relative to the TdT of SEQ ID NO: 1 or composition comprising the TdT of SEQ ID NO: 1, e.g., relative to a wild-type TdT polymerase, e.g., relative to one or more other modified TdT polymerases, etc.). In some embodiments, activity of a modified TdT polymerase or composition comprising the modified TdT polymerase is retained at higher temperatures (e.g., at 44° C., 48° C., 52° C., 54° C., and/or 55° C.), to at least a particular degree, relative to activity at 37° C. In some embodiments, activity is retained to at lower temperatures (e.g., below 37° C.), to at least a particular degree, relative to activity at 37° C.

Without wishing to be bound by theory, the present disclosure contemplates that, in some embodiments, improved activity (e.g. by a modified TdT polymerase) at certain temperatures or temperature ranges (e.g., higher temperatures) may reduce formation of any secondary structure(s) during synthesis.

In some embodiments, compositions provided by the present disclosure such as compositions comprising and/or using one or more linker-nucleotide components are able to be used to perform reactions at lower temperatures without concern for secondary structure formation and, thus, reactions may proceed at temperatures lower than, e.g., 55° C., 54° C., 52° C., 48° C., 44° C., 37° C., etc.

In some embodiments, polymerase activity of a modified TdT polymerase or composition comprising the modified TdT polymerase as provided herein shows polymerase activity that is at least about 60%, about 65%, about 70%, about 75%, about 80%, about 85%, about 90%, about 91%, about 92%, about 93%, about 94%, about 95%, about 96%, about 97%, about 98%, about 99%, or about 100% of polymerase activity of the TdT same or a different polymerase (e.g., including a reference TdT polymerase) at 37° C. In some embodiments, the activity of a modified TdT polymerase or composition comprising the modified TdT polymerase has activity that is greater than the polymerase activity of the same or different polymerase (e.g., including a reference TdT polymerase) including greater than the highest displayed (e.g. 100%) activity of a particular polymerase.

In some embodiments, the activity of a modified TdT polymerase or composition comprising the modified TdT polymerase has increased nucleotidyl transferase activity (e.g., relative to the TdT of SEQ ID NO: 1 or composition comprising the TdT of SEQ ID NO: 1, e.g., relative to one or more other modified TdT polymerases, etc.). In some embodiments, activity of a modified TdT polymerase or composition comprising a modified TdT polymerase as provided herein is assessed during a single nucleotide extension reaction by capillary electrophoresis. In some embodiments, a composition such as a conjugate, comprising a TdT polymerase (e.g., a modified TdT polymerase, e.g., a reference polymerase, e.g., the TdT of SEQ ID NO: 1) has polymerase activity. In some such embodiments, the present disclosure contemplates that such a composition can bind and facilitate an extension reaction by adding a linked nucleotide.

In some embodiments, a composition such as a conjugate, comprising a TdT polymerase (e.g., a modified TdT polymerase, e.g., a reference polymerase, e.g., the TdT of SEQ ID NO: 1) has substantially no bias for incorporating a oligonucleotide substrate having a nucleotide terminal end of A, T, G, or C into a sequence.

In some embodiments, incorporation time of a nucleotide extension reaction is between about 0.1 seconds and about 60 seconds. In some embodiments, the incorporation time of a nucleotide extension is less than 1 second. In some embodiments, the incorporation time of a nucleotide extension is less than 5 seconds. In some embodiments, the incorporation time of a nucleotide extension is less than 10 seconds. In some embodiments, the incorporation time of a nucleotide extension is less than 15 seconds. In some embodiments, the incorporation time of a nucleotide extension is less than 20 seconds. In some embodiments, the incorporation time of a nucleotide extension is less than 25 seconds. In some embodiments, the incorporation time of a nucleotide extension is less than 30 seconds. In some embodiments, the incorporation time of a nucleotide extension is less than 35 seconds. In some embodiments, the incorporation time of a nucleotide extension is less than 40 seconds. In some embodiments, incorporation time of a nucleotide extension reaction is less than 45 seconds. In some embodiments, incorporation time of a nucleotide extension reaction is less than 60 seconds. In some embodiments, incorporation time of a nucleotide extension reaction is less than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, or 0.1 seconds.

In some embodiments, extension reactions catalyzed by a cpTdT polymerase are faster than by a modified TdT polymerase. In some embodiments, speed of reaction can be assessed by amount of extension product remaining after a period of time. For example, in some embodiments, a reaction catalyzed using a cpTdT-based composition is faster than that of a modified TdT polymerase-based composition, as evidenced by increased total percentage of reaction products over a period of time. In some embodiments, the period of time is about 5 seconds or less (e.g., 5, 4, 3, 2, 1). In some embodiments, the period of time is about 60, 55, 50, 45, 40, 35, 30, 25, 20, 15, 10, 9, 8, 7, 6, 5 seconds or less. In some embodiments, the period of time is about 140, 135, 130, 125, 120, 115, 110, 105, 100, 95, 90, 85, 80, 75, 70, 65 seconds or less.

In some embodiments, a modified TdT polymerase or composition comprising a modified TdT polymerase (e.g., a conjugate) disclosed herein has increased protease resistance (e.g., relative to the TdT of SEQ ID NO: 1 or composition comprising the TdT of SEQ ID NO: 1, e.g., relative to one or more other modified TdT polymerases, etc.).

### Methods of Polynucleotide Synthesis

The present disclosure provides methods of nucleic acid synthesis such as with a TdT polymerase as provided herein. Nucleic acid synthesis can refer to synthesis, or generation of a product that is a nucleic acid molecule (i.e. a polynucleotide). Methods of nucleic acid synthesis can comprise stepwise synthesis, wherein nucleotides are inserted stepwise into a nucleic acid polymer or polynucleotide. By way of non-limiting example, one typical process for stepwise synthesis of a polynucleotide comprises adding nucleotides stepwise to a starter molecule (e.g., an initial oligonucleotide) via the cycled steps of: addition of a TdT polymerase (e.g., a modified TdT polymerase, e.g., a TdT polymerase of SEQ ID NO: 1) and a nucleotide either separately or as a TdT polymerase-nucleotide conjugate to an oligonucleotide and covalently incorporating the nucleotide to the 3′ end of the oligonucleotide catalyzed by the TdT polymerase. Successful incorporation of a nucleotide to an oligonucleotide can be referred to as an “extension” or “extension reaction.”

In some embodiments, a method of nucleic acid synthesis comprises providing a TdT polymerase (e.g., a modified TdT polymerase, e.g., a TdT polymerase of SEQ ID NO: 1) and a nucleotide and contacting a sample comprising a polynucleotide with the TdT polymerase (e.g., a modified TdT polymerase, e.g., a TdT polymerase of SEQ ID NO: 1) and the nucleotide.

In some embodiments, the TdT polymerase (e.g., a modified TdT polymerase, e.g., a TdT polymerase of SEQ ID NO: 1) comprises a sequence having at least 90%, at least 95%, at least 96%, at least 97%, at least 98%, at least 99% identity or up to 100% identity to that of SEQ ID NO: 1. In some embodiments, the polymerase (e.g., a modified TdT polymerase, e.g., a TdT polymerase of SEQ ID NO: 1) comprises a sequence having at least 60%, at least 65%, at least 70%, at least 75%, at least 80%, at least 85%, at least 90%, at least 95%, at least 96%, at least 97%, at least 98%, at least 99% identity, at least 99.1%, at least 99.2%, at least 99.3%, at least 99.4%, at least 99.5%, at least 99.6%, at least 99.7%, at least 99.8%, or at least 99.9% or more or up to 100% identity to that of SEQ ID NO: 1. In some embodiments, the TdT comprises or consists of the amino acid sequence of SEQ ID NO: 1. In some embodiments, a TdT polymerase is a modified TdT polymerase (e.g. a TdT variant, e.g., a chimeric TdT variant).

In some embodiments, the modified TdT polymerase (e.g., a TdT variant, e.g., a chimeric TdT variant) comprises or consists of a sequence selected from any one of SEQ ID NOs: 32-80, or a sequence having at least 90%, at least 95%, at least 96%, at least 97%, at least 98%, at least 99% at least 99.1%, at least 99.2%, at least 99.3%, at least 99.4%, at least 99.5%, at least 99.6%, at least 99.7%, at least 99.8%, or at least 99.9% or more identity thereto.

In some embodiments, the cpTdT polymerase derived from a parent TdT (e.g. a TdT variant, e.g., a chimeric TdT variant) comprises or consists of a sequence selected from any one of SEQ ID NOs: 88-120, or a sequence having at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% or more identity thereto.

In some embodiments, a modified TdT polymerase (e.g., a TdT variant, e.g., a chimeric TdT variant) and nucleotide are linked together (i.e. tethered) to form a conjugate (i.e. a polymerase-nucleotide conjugate). In stepwise synthesis using the conjugate, the tethered nucleotide is covalently incorporated (i.e., is added) into the 3′ end of the oligonucleotide, which is catalyzed by the tethered modified TdT polymerase (e.g., a TdT variant, e.g., a chimeric TdT variant). The tethered modified TdT polymerase (e.g., a TdT variant, e.g., a chimeric TdT variant) can stay tethered to the nucleotide following covalent incorporation. Covalent incorporation of a nucleotide can be referred to as an extension or an addition. The tethered modified TdT polymerase (e.g., a TdT variant, e.g., a chimeric TdT variant) can be cleaved from the inserted nucleotide to expose the 3′ end of the oligonucleotide. These steps can be repeated to synthesize a desired polynucleotide. The desired polynucleotide can have a pre-determined (i.e., pre-defined or target) sequence.

In some embodiments, the modified TdT polymerase (e.g. a TdT variant, e.g., a chimeric TdT variant) and nucleotide are added to the extension reaction as separate species. In some embodiments, the nucleotide is a nucleotide analog. In some embodiments, the nucleotide analog is a reversible terminator. Reversible terminators are known in the art for use in nucleic acid synthesis. Uses of reversible terminators in nucleic acid synthesis have been described previously; see, for example, WO 2021/122539 A1, WO 2018/215803 A1, WO 2021/094251 A1, and WO 2020/081985 A1.

In some embodiments, methods of nucleic acid synthesis as provided herein are carried out in a reaction buffer composition. In some embodiments, the reaction buffer composition is an aqueous solution. In some embodiments, the reaction buffer composition comprises a set of components suitable for the stability of the polymerase, nucleotide, polymerase-nucleotide conjugates, starter molecule, nucleic acid molecule products, and any surface or matrix on which the methods disclosed herein are carried out. In some such embodiments, the reaction buffer composition comprises a set of components suitable for carrying out catalytic steps (e.g., polynucleotide polymerization performed by a polymerase) described in methods of nucleic acid synthesis in accordance with the present disclosure.

The conditions under which nucleic acid synthesis is carried out can be varied. For example, the amounts of times for carrying out each step in a stepwise nucleotide addition cycle can be varied to improve the purity of a plurality of products generated by the methods of nucleic acid synthesis described herein.

In some embodiments, methods of nucleic acid synthesis in accordance with the present disclosure generate a nucleic acid molecule product (i.e., a polynucleotide product). In some embodiments, the nucleic molecule product (i.e., polynucleotide product) has a target (i.e., pre-determined) sequence. A “target” or “pre-determined” sequence refers to a desired polynucleotide sequence that is intentionally produced by the method of nucleic acid synthesis. The pre-determined sequence can include any number of nucleotides comprising a nucleobase (e.g., adenine, thymine, guanine, cytosine, and/or uracil). In some embodiments, the nucleotide is a modified nucleotide (i.e., a nucleotide analog). In some embodiments, the nucleobase is a modified nucleobase. In some embodiments, the pre-determined sequence contains one or more designated positions which may be a random nucleobase. Inclusion of a position with a random nucleobase can be useful, for example, in introducing randomized mutation into a polynucleotide product.

A nucleic acid molecule product or polynucleotide product generated by the methods described herein can contain a plurality of products. In some embodiments, the plurality of products comprises a nucleic acid molecule comprising the target (i.e., pre-determined) sequence. In some embodiments, the plurality of products comprises a nucleic acid molecule comprising a sequence that is not the target sequence. In some embodiments, the plurality of products comprises a nucleic acid molecule product comprising the target sequence and a nucleic acid molecule product that is not the target sequence. The “purity” of the plurality of products can refer to the ratio of the abundance of nucleic acid molecule products with the target sequence to the abundance of nucleic acid molecule products that do not have the target sequence. The purity of a product can be assessed by any number of methods known in the art for determining the sequence of a nucleic acid. Any suitable nucleic acid sequencing method can be used. For example, the product can be assessed, without limitation, by Sanger sequencing, next generation sequencing (e.g., Illumina sequencing), or long-read sequencing (e.g. small molecule, real-time sequencing (SMRT) and nanopore sequencing).

In some embodiments, a method of nucleic acid synthesis in accordance with the present disclosure produces a product having a purity between about 10% and about 99.99%. In some embodiments, the method of nucleic acid synthesis produces a product having a purity of at least 10%. In some embodiments, the method of nucleic acid synthesis produces a product having a purity of at least 10%. In some embodiments, the method of nucleic acid synthesis produces a product having a purity of at least 20%. In some embodiments, the method of nucleic acid synthesis produces a product having a purity of at least 30%. In some embodiments, the method of nucleic acid synthesis produces a product having a purity of at least 40%. In some embodiments, the method of nucleic acid synthesis produces a product having a purity of at least 50%. In some embodiments, the method of nucleic acid synthesis produces a product having a purity of at least 60%. In some embodiments, the method of nucleic acid synthesis produces a product having a purity of at least 70%. In some embodiments, the method of nucleic acid synthesis produces a product having a purity of at least 80%. In some embodiments, the method of nucleic acid synthesis produces a product having a purity of at least 90%. In some embodiments, the method of nucleic acid synthesis produces a product having a purity of at least 95%. In some embodiments, the method of nucleic acid synthesis produces a product having a purity of at least 99%.

### EQUIVALENTS, SCOPE & OTHER EMBODIMENTS

Those skilled in the art will recognize or be able to ascertain using no more than routine experimentation, many equivalents to the specific embodiments in accordance with the compositions and methods described herein. The scope of the present disclosure is not intended to be limited to the above Description, but rather is as set forth in the appended claims.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

It is to be understood that the words which have been used are words of description rather than limitation, and that changes may be made within the purview of the appended claims without departing from the true scope and spirit of the disclosure in its broader aspects.

Section and table headings materials, methods, and examples are illustrative only and not intended to be limiting.

All cited sources, for example, references, publications, including patents or patent applications, databases, database entries, and art cited herein, are incorporated into this application by reference in their entireties, even if not expressly stated in the citation. In case of conflicting statements of a cited source and the present application, the statement in the present application shall control.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18^{th} Edition (Easton, Pennsylvania: Mack Publishing Company, 1990); Carey and Sundberg Advanced Organic Chemistry 3^{rd} Ed. (Plenum Press) Vols A and B(1992).

### Example 1: Production, Purification, and Characterization of Modified TdT Polymerases

### TdT Variant Expression

Three exemplary TdT variants of SEQ ID NOs: 32, 36, and 37 were expressed in BL21 DE3 Gold E. coli competent cells (Agilent Technologies, www.agilent.com/cs/library/usermanuals/public/230130.pdf). Cells were thawed and incubated with plasmid capable of expressing a TdT variant and an ampicillin resistance gene for 5 minutes on ice. Cells were plated onto LB agarose and incubated overnight at 37° C. Colonies were picked and incubated overnight at 37° C. in 20 mL of 2×YT media containing ampicillin. Resulting cultures were used to inoculate a 1 L flask of Terrific Broth media (expression culture) and allowed to incubate at 37° C. with shaking. After 4 hours of incubation, temperature was lowered to 16° C. Following 5 hours of incubation at 16° C., expression of the TdT variant was induced by adding IPTG at a final concentration of 0.5 mM and cultures were allowed to incubate overnight at 16° C. Cultures were collected and centrifuged 6000×g for 20 minutes at 4° C. Pellets were resuspended in 30 mL lysis buffer (20 mM Tris-HCl, 0.5 M NaCl, pH 8.0, 5 mM Imidazole), and lysozyme was added and the suspension was mixed and allowed to incubate for 45 minutes with rocking at room temperature. Cell lysate was frozen at −80° C.

### Purification of TdT Variants

TdT variants were purified using Ni-NTA resin. Cell lysate was thawed for 30 minutes at 32° C. and then placed on ice. Lysates were sonicated and centrifuged at 4° C. for 15 minutes at 30,000×g, supernatant (cleared lysate) was collected and centrifuged again for 20 minutes at 30,000×g. Ni-NTA resin slurry was washed according to manufacturer's protocol and cleared lysate was incubated with slurry for 45 minutes at 4° C. The slurry was loaded onto a gravity column at 4° C. Slurry was washed twice with 150 mL of wash buffer (20 mM Tris-HCl, 1 M NaCl, pH 8.0, 40 mM Imidazole). TdT variants were eluted with 36 mL of elution buffer (20 mM TrisHCl pH 8.0, 25 mM NaCl, 250 mM Imidazole). Glycerol was added to the eluent at a final concentration of 5% (v/v), and then eluent was flash frozen.

Frozen eluents containing the individual TdT variants were thawed and purified by anion exchange FPLC. A Q anion exchange column was equilibrated and loaded with eluent containing the TdT variant in potassium phosphate buffer, pH 6.5 (buffer A). The TdT variant was eluted in a 0-30% gradient of elution buffer B (potassium phosphate, 1 M NaCl, pH 6.5) over 15 column volumes. Fractions containing the TdT variant were collected and concentrated using a 30 kDa molecular weight cut off filter. Protein sample purity was analyzed by SDS-PAGE and concentration was determined by absorption at 280 nm.

### Characterization of TdT Variants

The polymerase activity of the TdT variants of SEQ ID NOs: 32, 36, and 37 was tested at a range of temperatures. Each TdT variant was incubated with 1 mM dATP, polymerase metal mix, Sybr Gold, and an initiator oligonucleotide at 37° C., 38° C., 40C, 44° C., 48° C., 52° C., 54° C., or 55° C. The reaction was allowed to proceed for about 90 seconds and monitored at 537 nm using a fluorometer. Increased fluorescence at excitation 495 nm/emission 537 nm is representative of nucleotide incorporation onto the initiator oligonucleotide catalyzed by the TdT variant, and is representative of TdT activity.

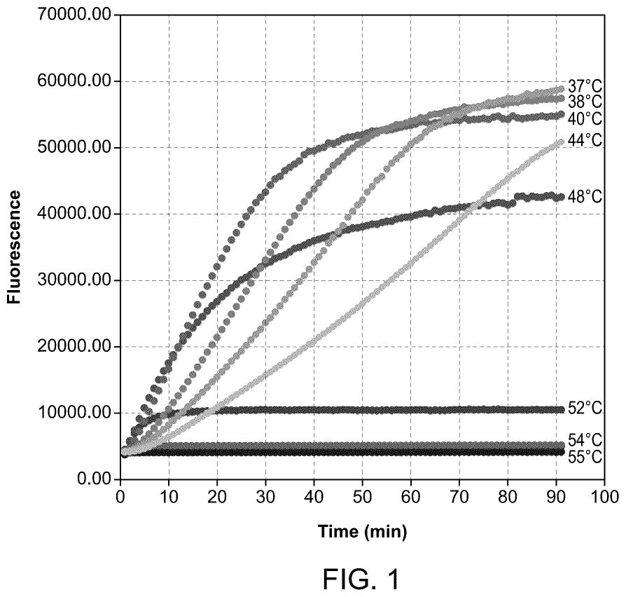

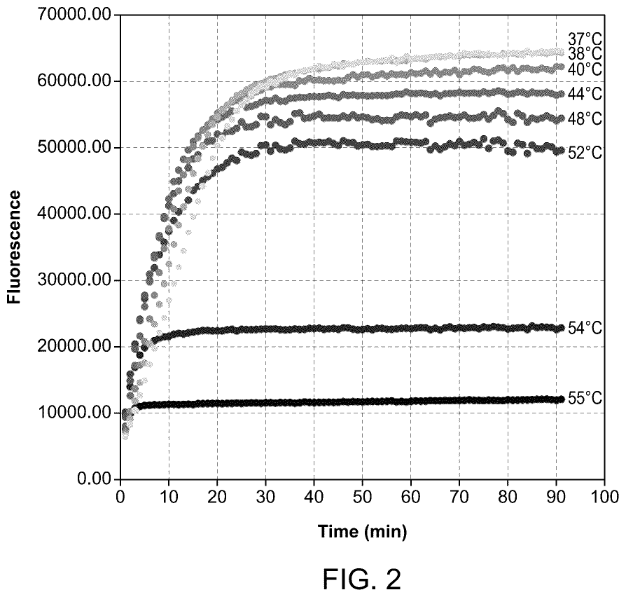

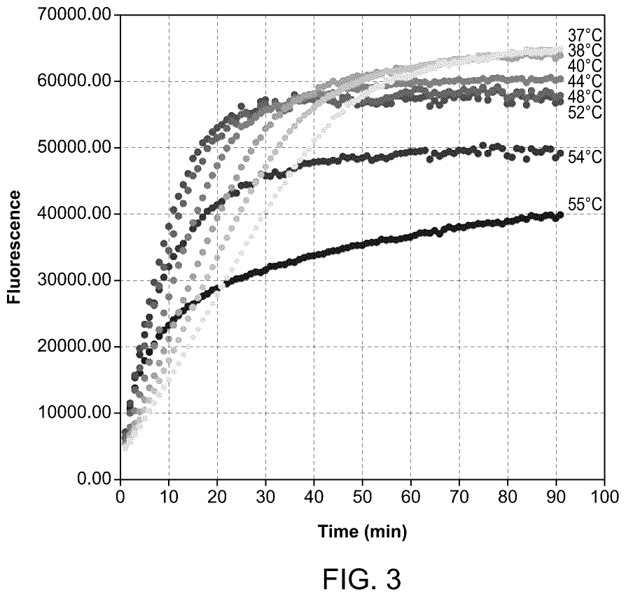

The results are shown as plots of fluorescence (y-axis) measured over time (x-axis) in FIG. 1 (with the polymerase of SEQ ID NO: 32); FIG. 2 (with the polymerase of SEQ ID NO: 36); and FIG. 3 (with the polymerase of SEQ ID NO: 37). Each of the three exemplary TdT variants showed polymerase activity and showed unique thermostability profiles (as compared to one another), where thermostability is indicated by retaining activity at higher temperatures and the more activity retained as temperature increases, the higher and more improved thermostability is relative.

### Example 2: Characterization of Modified TdT Polymerase Conjugates

Conjugation of TdT Variants and dNTPs Two exemplary TdT variants were conjugated to nucleotide analogs. The first TdT variant (SEQ ID NO: 74) was conjugated to dTTP (conjugate referred to as TdT-dATP), dGTP (conjugate referred to as TdT-dGTP), and dCTP (conjugate referred to as TdT-dCTP). The second TdT variant (SEQ ID NO: 42) was conjugated to dATP (TdT-dATP).

To prepare the conjugates, nucleotide-analog having a sulfide reactive linker (linker-nucleotide, about 1.65 mM) was reacted with the TdT chimeric variant (about 120 μM) overnight at 4° C. on a rotating platform. TdT chimeric variant-nucleotide conjugate was purified using a Superdex 200 size exclusion column using FPLC. Fractions containing the conjugate collected and the conjugate was concentrated to 2 mg/mL in TP8 buffer with 0.1% polysorbate 20.

### Characterization of TdT Variant-Nucleotide Conjugates

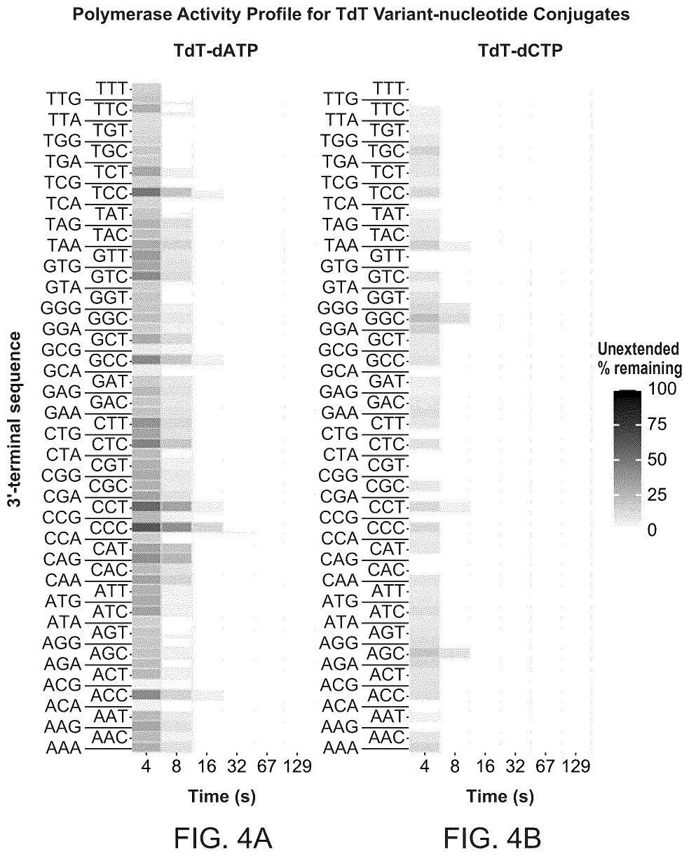

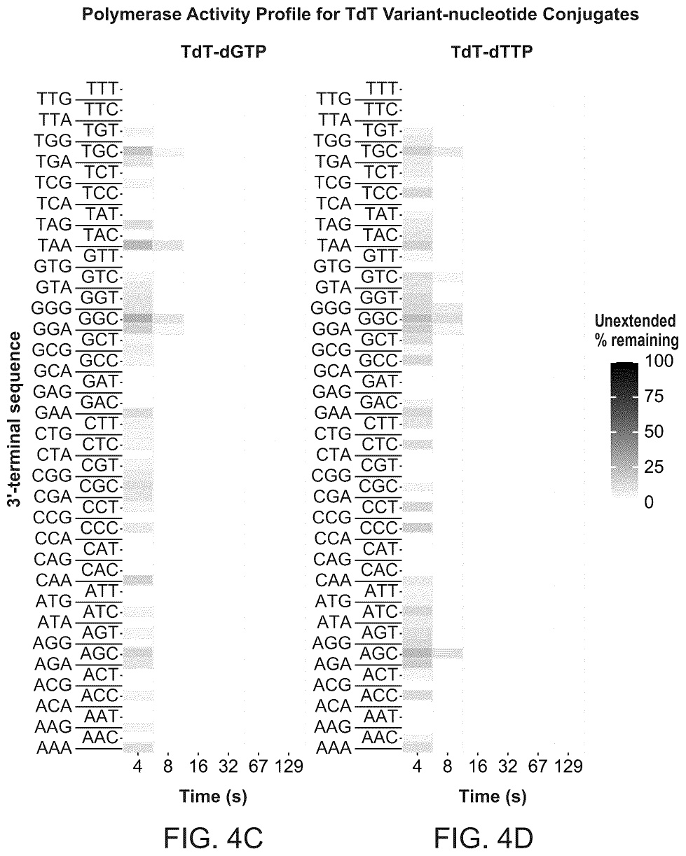

Polymerase activity for each of the conjugates was determined. A single nucleotide extension reaction was carried out by each conjugate on a panel of oligo substrates, each oligo substrate having a different 3 nucleotide terminal end, representing all combinations of trimer substrate to which the TdT chimeric variant conjugate can bind and facilitate an extension reaction by adding the linked nucleotide. Extensions were carried out in reaction buffer (20 mM Tris Acetate, 50 mM Potassium Acetate, 250 μM cobalt acetate, 50 nM DNA oligo substrate, 1 μM TdT enzyme-nucleotide conjugate, pH 7.9) for up to 129 seconds. Extension reactions were analyzed by capillary electrophoresis, and quantified as described previously (see Palluk et al. Nature Biotechnology. 36(7):645-650, 2018). The results are represented as heat map plots showing polymerase activity profiles of TdT-variant nucleotide conjugates (FIGS. 4A-4D). This analysis both confirms that polymerase activity is maintained in conjugates having the TdT chimeric variants. In addition, the present example provides insight into potential biases that a TdT chimeric variant-nucleotide conjugate may have for incorporating a given nucleotide into particular trimer sequences. Remarkably, the conjugates incorporated their respective nucleotides (FIG. 4A, dATP; FIG. 4B, dCTP; FIG. 4C, dGTP; and FIG. 4D, dTTP) into every possible 3-nucleotide terminal and all incorporation times occurred in under 45 seconds, indicating that the TdT chimeric variant-nucleotide conjugates have polymerase activity, utility in polynucleotide extension reactions, and have little, if any, bias toward any particular trimer sequence on which the linked nucleotide is incorporated.

### Example 3: Characterization of cpTdT Polymerase Conjugates

An exemplary cpTdT having a sequence set forth in SEQ ID NO: 90 was designed from a parent TdT (SEQ ID NO: 42) that is a modified TdT, expressed, and isolated as described in Example 1. The exemplary circular permutation variant of a modified TdT polymerase (cpTdT) was designed (SEQ ID NO: 90) was derived from the modified TdT polymerase of SEQ ID NO: 42.

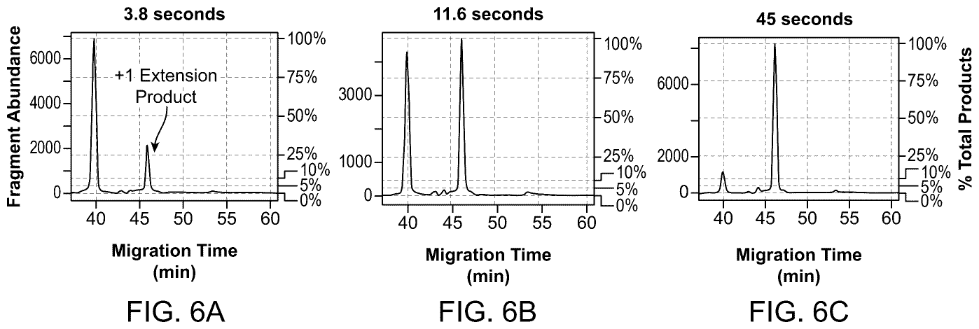

The cpTdT was tethered to a dTTP as described in Example 2 to prepare a cpTdT-nucleotide conjugate. Extension reactions were carried out in reaction buffer (20 mM Tris Acetate, 50 mM Potassium Acetate, 500 μM cobalt acetate, 50 nM DNA oligo substrate, 1 μM cpTdT enzyme-nucleotide conjugate, pH 7.9) for up to 45 seconds. Extension reaction products were analyzed by capillary electrophoresis and quantified as described previously (see Palluk et al. Nature Biotechnology. 36(7):645-650 2018). FIGS. 6A-6C show exemplary results of these reactions as plots of fragment abundance and total percentage of reaction products (left and right y-axes, respectively) measured over time (x-axis) at 3.8, 11.6, and 45 seconds in FIGS. 6A, 6B, and 6C, respectively.

The results show that the +1 extension product increased as a total percentage of reaction products over time. These results demonstrate that the cpTdT has template-independent polymerase activity and can be used to extend an oligonucleotide by a nucleotide. Furthermore, the data demonstrate that the cpTdT can incorporate a tethered nucleotide when used as a cpTdT-nucleotide conjugate.

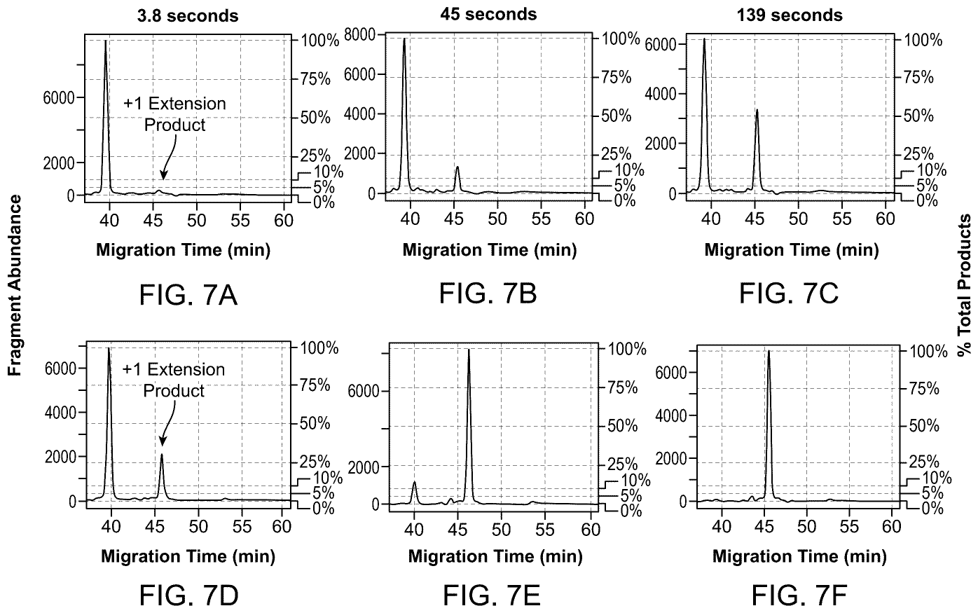

The polymerase activity of an exemplary cpTdT (SEQ ID NO: 90) was also compared to its parent (i.e., the modified TdT polymerase of SEQ ID NO: 42 that has not been circularly permuted or had its amino acid sequence truncated or otherwise modified relative to what is set forth in SEQ ID NO: 42) under the same reaction conditions. Analyses of the reaction products after 3.8 seconds, 45 seconds, and 139 seconds were plotted as fragment abundance and percentage of total reaction products (left and right y-axes, respectively) measured over time (x-axis) in FIGS. 7A-7F. The results show that for both the modified TdT polymerase without the circular permutation (FIGS. 7A-7C) and the cpTdT (FIGS. 7D-7F), the +1 extension product increased as a total percentage of reaction products over time. Surprisingly, the extension reactions catalyzed by the cpTdT (FIGS. 7D-7F) for the modified TdT polymerase was remarkably faster than those catalyzed by the modified TdT polymerase (FIGS. 7A-7C), as evidenced by the amount of extension product present after 139 seconds (50%) as seen in FIGS. 7C and 7F. In contrast, for the cpTdT, about 100% of the extension product was present after 139 seconds. These results show that the cpTdT was able to catalyze the extension reaction at a much faster rate than its parental TdT (i.e., a modified TdT polymerase not having a circular permutation).

1. A modified terminal deoxynucleotidyl transferase (TdT) polymerase comprising an amino acid sequence: (i) having at least 90% identity to that of the TdT polymerase of SEQ ID NO: 1; and (ii) having one or more amino acid substitutions relative to the amino acid sequence of the TdT polymerase of SEQ ID NO: 1.

2. The modified TdT polymerase of claim 1, wherein the one or more amino acid substitutions is selected from the group consisting of A52C, D54E, D54C, D60A, L62F, L62C, S68R, L70C, S76A, S76C, T92L, I95L, S104R, G108E, I110L, K131C, F148Y, F166L, D206G, M211L, T235G, C36V, E64G, A118V, K157E, Q241R, C285A, E338N, I158V, P187S, D260N, S319P, M73Q, E145K, S163T, Q168E, P187A, Q271L, C285G, G294Q, M352L, L262A, R335T, V7D, L175K, N185T, M194V, K300E, R361K, E368G, E58C, C183G, M73C, R217C, K90E, K101E, K119M, R364E, D280A, D280N, D354A, D354L, H356V, H356A, L141C, D277C, A278C, R335C, R339C, R342C, A69C, T134C, E61C, S32P, M194T, E368K, R339W, T346N, H347Q, S163S, E66C, R361K, K141C, and combinations thereof.

3. The modified TdT polymerase of claim 1, wherein the one or more amino acid substitutions comprises C36V, G108E, A118V, and/or K157E.

4. The modified TdT polymerase of claim 3, wherein the one or more amino acid substitutions further comprises V7D, L62F, T92L, E145K, F148Y, I158V, Q168E, L175K, C183G, N185T, Q271L, G294Q, K300E, S319P, and/or M352L.

5-29. (canceled)

30. The modified TdT polymerase of claim 1, wherein the sequence of the modified TdT polymerase comprises at least one active site motif corresponding to a homologous nucleotidyl transferase from a species selected from the group comprising Tinamus guttatus (SEQ ID NO: 5), Eudromia elegans (SEQ ID NO: 6), Nothoprocta perdicaria (SEQ ID NO: 7), Nothoprocta ornate (SEQ ID NO: 8), Nothoprocta pentlandii (SEQ ID NO: 9), Nothocercus Julius (SEQ ID NO: 10), Nothocercus nigrocapillus (SEQ ID NO: 11), Crypturellus undulatus (SEQ ID NO: 12), Crypturellus soui (SEQ ID NO: 13), Serilophus lunatus (SEQ ID NO: 14), Gouania willdenow (SEQ ID NO: 15), Silurus meridionalis (SEQ ID NO: 16), Coregonus clupeaformis (SEQ ID NO: 17), Oncorhynchus nerka (SEQ ID NO: 18), Vicugna pacos (SEQ ID NO: 19), Elephantulus edwardii (SEQ ID NO: 20), Galeopterus variegatus (SEQ ID NO: 21), Engystomops pustulosus (SEQ ID NO: 22), Bufo bufo (SEQ ID NO: 23), Bufo gargarizans (SEQ ID NO: 24), Sciurus carolinensis (SEQ ID NO: 25), Nanorana perkeri (SEQ ID NO: 26), Carassius auratus (SEQ ID NO: 27), Pimephales promelas (SEQ ID NO: 28), Chanos chanos (SEQ ID NO: 29), Python bivittatus (SEQ ID NO: 30), and Danio rerio (SEQ ID NO: 31), wherein the active site motif corresponds to positions 332-335 of the TdT polymerase of SEQ ID NO: 1.

31. The modified TdT polymerase of claim 30, wherein the active site motif is selected from the group consisting of TGSK (SEQ ID NO: 121), TGSP (SEQ ID NO: 122), TGSQ (SEQ ID NO: 123), and TGST (SEQ ID NO: 124).

32. A modified TdT polymerase of claim 1, wherein the modified TdT polymerase is a chimeric TdT variant polymerase comprising a sequence having at least 90% identity to SEQ ID NO: 1, and an active site motif corresponding to positions 332-335 of SEQ ID NO: 1 comprising an active site motif identical to an active site motif from a homologous nucleotidyl transferase, wherein the homologous nucleotidyl transferase comprises a sequence selected from the group comprising SEQ ID NOs: 5-31.

33. The chimeric TdT variant polymerase of claim 32, wherein the active site motif is selected from the group consisting of TGSK (SEQ ID NO: 121), TGSP (SEQ ID NO: 122), TGSQ (SEQ ID NO: 123), and TGST (SEQ ID NO: 124).

34. The modified TdT polymerase of claim 1, wherein the modified TdT polymerase comprises one or more improved properties compared to the TdT polymerase of SEQ ID NO: 1.

35. The modified TdT polymerase of claim 34, wherein the one or more improved properties comprises increased thermostability, increased nucleotidyl transferase activity, and increased protease resistance.

36. The modified TdT polymerase of claim 1, wherein the amino acid sequence of the TdT polymerase comprises a sequence having at least 80%, at least 85%, at least 90%, at least 95%, at least 98%, at least 99%, or 100% identity to an amino acid sequence selected from any of SEQ ID NOs: 32-80.

37. The modified TdT polymerase of claim 36, wherein the modified TdT polymerase is a chimeric variant polymerase.

38. A polynucleotide comprising a sequence encoding the modified TdT polymerase of claim 1.

39. The polynucleotide of claim 38, wherein the sequence encoding the modified TdT polymerase is codon optimized.

40. A vector comprising the polynucleotide of claim 38.

41. A host cell comprising polynucleotide of claim 38.

42. A method of producing a modified TdT polymerase comprising, culturing the host cell of claim 41 under appropriate conditions for expressing the modified TdT polymerase; and isolating the modified TdT polymerase.

43. A conjugate comprising the modified TdT polymerase of claim 1, a linker, and a nucleotide, wherein the linker tethers the nucleotide to the modified TdT polymerase.

44-45. (canceled)

46. A method of nucleic acid synthesis, comprising:
(a) providing a sample comprising a polynucleotide;
(b) providing the modified TdT polymerase of claim 1 and a nucleotide; and
(c) contacting the sample comprising the polynucleotide with the modified TdT polymerase and nucleotide,
wherein the modified TdT polymerase catalyzes an extension reaction comprising a covalent addition of the nucleotide onto the 3′ hydroxyl of said polynucleotide.

47-56. (canceled)

57. A circularly permuted polypeptide comprising template-independent polymerase activity and comprising an amino acid sequence derived from a parental polypeptide.

58-135. (canceled)
